(19) United States
(12) Patent Application Publication
(10) Pub. No.: US 2021/0269547 A1
(43) Pub. Date: Sep. 2, 2021

(54) ANTIBODY TUMOR-TARGETING ASSEMBLY COMPLEXES

(71) Applicant: THE GENERAL HOSPITAL CORPORATION, Boston, MA (US)

(72) Inventors: Mark COBBOLD, Boston, MA (US); David MILLAR, Boston, MA (US)

(73) Assignee: THE GENERAL HOSPITAL CORPORATION, Boston, MA (US)

(21) Appl. No.: 17/257,391

(22) PCT Filed: Jul. 2, 2019
(86) PCT No.: PCT/US2019/040336

§ 371 Date: Dec. 31, 2020

Related U.S. Application Data
(60) Provisional application No. 62/693,125, filed on Jul. 2, 2018.

(51) Int. Cl. C07K 16/30; C07K 16/28

(52) U.S. Cl. CPC C07K 16/30; C07K 16/2809; C07K 2317/56; C07K 16/2812; C07K 2317/31; C07K 16/2815

(57) ABSTRACT

The present disclosure provides antibody tumor-targeting assembly complexes (ATTACs) for selectively activating desired immune cells in the tumor microenvironment.

### CROSS-REFERENCE TO RELATED APPLICATIONS

This application is a 371 National Phase Entry of International Patent Application No. PCT/US2019/040336 filed Jul. 2, 2019, which claims benefit under 35 U.S.C. § 119(e) of U.S. Provisional Application No. 62/693,125 filed Jul. 2, 2018, the contents of which are incorporated herein by reference in their entirety.

### DESCRIPTION

### Field

This application relates to targeted immune cell engaging agents for treating cancer.

### SEQUENCE LISTING

The instant application contains a Sequence Listing which has been submitted electronically in ASCII format and is hereby incorporated by reference in its entirety. Said ASCII copy, created on Dec. 4, 2020, is named 030258-092180WOPT_Sequence_Listing.txt and is 71,065 bytes in size.

### BACKGROUND

Cancer creates significant loss of life, suffering, and economic impact. Immunotherapeutic strategies for targeting cancer have been an active area of translational clinical research.

A variety of other approaches have been explored for immunotherapy, but many of these prior approaches lack sufficient specificity to particular cancer cells. For example, demibodies have been designed each having an scFv portion binding to different antigens on a target cell, an Fc domain allowing pairing to a complementary demibody, and a binding partner capable of forming an association to another binding partner on a complementary demibody. WO 2007/062466. These demibodies, however, are not necessarily specific to cancer cells and could bind and have activity on other cells expressing the same antigens. See also WO 2013/104804, which provides a first polypeptide with a targeting moiety binding to a first antigen and a first fragment of a functional domain, along with a second polypeptide with a targeting moiety binding to a second antigen and a second fragment of a functional domain that is complementary to the first fragment of the functional domain. Likewise, this approach is not necessarily specific to cancer cells and could bind and have activity on other cells expressing the same antigens.

Bispecific T-cell Engaging Antibodies (BiTEs) have been proposed by others; however, these constructs are often not sufficiently specific to the tumor environment. Additionally, BiTEs also can activate regulatory T cells (Tregs), promoting undesired Treg activity at the tumor site. For example, stimulating Tregs has been associated, in certain patients, with high levels of proliferation of suppressive Tregs and rapid cancer progression, termed hyperprogressive disease (see Kamada et al., PNAS 116(20):9999-10008 (2019)). Specific instances of hyperprogressive disease have been seen in patients treated with anti-PD-1 antibodies, which activates and expands certain tumor-infiltrating PD-1+ Treg cells, but concerns exist that other means of stimulating Tregs could have similar unwanted effects in a minority of patients.

Other approaches employing more specificity so that T cells are targeted to cancer cells do not have any means for selecting which T cells arrive at or are activated at the site of the cancer. WO 2017/087789. Activating all T cells, including T cells that do not benefit an immunooncology approach for treating the patient's cancer.

There are two problems with the current bi-specific antibody approach of activating T cells via CD3. The first of these is the over-activation of the immune response. Although not widely discussed, these agents are incredibly potent and are given at extremely low doses compared with whole antibody therapies. This will be partly due to the fact that these reagents can theoretically activate every T cell by binding to CD3. When someone has a viral infection, around 1-10% of their T cells are activated and they feel lethargic and ill because of the immune response. When more T cells are activated, this can lead to larger problems including cytokine release syndrome (CRS) and death in rare cases. CRS can be triggered by release of cytokines from cells targeted by biologics, as well as by cytokine release from recruited immune effector cells. Therefore, there is a need to limit the total number of T cells that are activated using these systems.

The second problem with current BiTE therapies is the CD3-specific activation of any T cell that is in the vicinity of the BiTE-bound target cell. Many immune cells respond to CD3 activation, including CD4 T cells (helper, regulatory, TH17, etc) and CD8 T cells, depending on which cells bind to the BiTE. This may mean that the efficacy of the BiTE is lost because activation of unwanted T cells such as regulatory T cells and TH17 T cells, inhibiting the cytolytic function of T cells such as CD8 T cells and cytotoxic CD4 T cells. Therapies could also be improved if they only activated particular types of T cells, such as only activating CD8+ T cells. The art has not previously proposed a solution to this problem. Only with this invention have we discovered the benefit of a system whereby the tumor-targeting was present to provide specificity for the unwanted and a second moiety was present to selectively bind to desirable immune cells which could combine at the site of the unwanted cancer cells and kill them.

### SUMMARY

In accordance with the description, this application describes agents and methods of treatment of cancer using antibody tumor-targeting assembly complexes (ATTACs).

In some embodiments, an agent for treating cancer in a patient comprises: (a) a first component comprising a targeted immune cell binding agent comprising: (i) a targeting moiety capable of targeting the cancer; and (ii) a first immune cell engaging domain capable of immune engaging activity when binding a second immune cell engaging domain, wherein the second immune cell engaging domain is not part of the first component; (b) a second component comprising a selective immune cell binding agent comprising: (i) an immune cell capable of selectively targeting an immune cell; and (ii) a second immune cell engaging domain capable of immune cell engaging activity when binding the first immune cell engaging domain, wherein the first and second immune cell engaging domains are capable of binding when neither is bound to an inert binding partner, wherein at least one of the first immune cell engaging domain or the second immune cell engaging domain is bound to an inert binding partner such at the first and second immune cell engaging domains are not bound to each other unless the inert binding partner is removed; and further comprising a cleavage site separating the first inert binding partner and the immune cell engaging domain to which it binds, wherein the cleavage site is: (i) cleaved by an enzyme expressed by the cancer cells; (ii) cleaved through a pH-sensitive cleavage reaction inside the cancer cell; (iii) cleaved by a complement-dependent cleavage reaction; or (iv) cleaved by a protease that is colocalized to the cancer cell by a targeting moiety that is the same or different from the targeting moiety in the agent.

In some embodiments, the first component is not covalently bound to the second component. In some embodiments, the first component is covalently bound to the second component.

In some embodiments, the immune cell engaging domains, when bound to each other, are capable of binding an antigen expressed on the surface of the immune cell. In some embodiments, the immune cell selection moiety capable of selectively targeting an immune cell selectively targets a T cell, a macrophage, a natural killer cell, a neutrophil, an eosinophil, a basophil, a γδ T cell, a natural killer T cell (NKT cells), or an engineered immune cell.

In some embodiments, the immune cell selection moiety capable of selectively targeting an immune cell selectively targets a T cell. In some embodiments, the T cell is a cytotoxic T cell. In some embodiments, the cytotoxic T cell is a CD8+ T cell. In some embodiments, the T cell is a helper T cell. In some embodiments, the helper T cell is a CD4+ T cell. In some embodiments, the immune cell selection moiety targets CD8, CD4, or CXCR3. In some embodiments, the immune cell selection moiety does not specifically bind regulatory T cells. In some embodiments, the immune cell selection moiety does not specifically bind TH17 cells. In some embodiments, the immune cell engaging domains, when bound to each other, are capable of binding CD3. In some embodiments, the immune cell engaging domains, when bound to each other, are capable of binding TCR.

In some embodiments, the immune cell selection moiety capable of selectively targeting an immune cell selectively targets a natural killer cell. In some embodiments, the immune cell selection moiety targets CD2 or CD56. In some embodiments, the immune cell engaging domains, when bound to each other, are capable of binding NKG2D, CD16, NKp30, NKp44, NKp46 or DNAM.

In some embodiments, the immune cell selection moiety capable of selectively targeting an immune cell selectively targets a macrophage. In some embodiments, the immune cell selection moiety targets CD14, CD11b, or CD40. In some embodiments, the immune cell engaging domains, when bound to each other, are capable of binding CD89 (Fc alpha receptor 1), CD64 (Fc gamma receptor 1), CD32 (Fc gamma receptor 2A) or CD16a (Fc gamma receptor 3A).

In some embodiments, the immune cell selection moiety capable of selectively targeting an immune cell selectively targets a neutrophil. In some embodiments, the immune cell selection moiety targets CD15. In some embodiments, the immune cell engaging domains, when bound to each other, are capable of binding CD89 (FcαR1), FcγRI (CD64), FcγRIIA (CD32), FcγRIIIA (CD16a), CD11b (CR3, αMβ2), TLR2, TLR4, CLEC7A (Dectin1), formyl peptide receptor 1 (FPR1), formyl peptide receptor 2 (FPR2), or formyl peptide receptor 3 (FPR3).

In some embodiments, the immune cell selection moiety capable of selectively targeting an immune cell selectively targets an eosinophil. In some embodiments, the immune cell selection moiety targets CD193, Siglec-8, or EMR1. In some embodiments, the immune cell engaging domains, when bound to each other, are capable of binding CD89 (Fc alpha receptor 1), FcεRI, FcγRI (CD64), FcγRIIA (CD32), FcγRIIIB (CD16b), or TLR4.

In some embodiments, the immune cell selection moiety capable of selectively targeting an immune cell selectively targets a basophil. In some embodiments, the immune cell selection moiety targets 2D7, CD203c, or FcεRIα. In some embodiments, the immune cell engaging domains, when bound to each other, are capable of binding CD89 (Fc alpha receptor 1) or FcεRI.

In some embodiments, the immune cell selection moiety capable of selectively targeting an immune cell selectively targets a γδ T cell. In some embodiments, the immune cell selection moiety targets γδ TCR. In some embodiments, the immune cell engaging domains, when bound to each other, are capable of binding γδ TCR, NKG2D, CD3 Complex (CD3ε, CD3γ, CD3δ, CD3ζ, CD3η), 4-1BB, DNAM-1, or TLRs (TLR2, TLR6).

In some embodiments, the immune cell selection moiety capable of selectively targeting an immune cell selectively targets a natural killer T cell. In some embodiments, the immune cell selection moiety targets Vα24 or CD56. In some embodiments, the immune cell engaging domains, when bound to each other, are capable of binding αβTCR, NKG2D, CD3 Complex (CD3ε, CD3γ, CD3δ, CD3ζ, CD3η), 4-1BB, or IL-12R.

In some embodiments, the immune cell selection moiety capable of selectively targeting an immune cell selectively targets an engineered immune cell. In some embodiments, the engineered immune cell is a CAR T cell, natural killer cell, natural killer T cell, or γδ T cell. In some embodiments, the immune cell selection moiety targets the CAR or a marker expressed on the immune cell. In some embodiments, the immune selection moieties targets LNGFR or CD20. In some embodiments, the immune cell engaging domains, when bound to each other, are capable of binding an antigen expressed by the engineered immune cell. In some embodiments, the antigen expressed by the engineered immune cell is CD3.

In some embodiments, the immune cell selection moiety comprises an antibody or antigen-specific binding fragment thereof. In some embodiments, the antibody or antigen-specific binding fragment thereof specifically binds an antigen on a T cell. In some embodiments, the antibody or antigen-specific binding fragment thereof specifically binds an antigen on a cytotoxic or helper T cell. In some embodiments, the antibody or antigen-specific binding fragment thereof specifically binds an antigen on a macrophage. In some embodiments, the antibody or antigen-specific binding fragment thereof specifically binds an antigen on a natural killer cell. In some embodiments, the antibody or antigen-specific binding fragment thereof specifically binds an antigen on a neutrophil. In some embodiments, the antibody or antigen-specific binding fragment thereof specifically binds an antigen on an eosinophil. In some embodiments, the antibody or antigen-specific binding fragment thereof specifically binds an antigen on a γδ T cell. In some embodiments, the antibody or antigen-specific binding fragment thereof specifically binds an antigen on a natural killer T cell. In some embodiments, the antibody or antigen-specific binding fragment thereof specifically binds an antigen on an engineered immune cell. In some embodiments, the engineered immune cell is a CAR T cell, natural killer cell, natural killer T cell, or γδ T cell.

In some embodiments, the immune selection moiety comprises an aptamer. In some embodiments, the aptamer specifically binds an antigen on a T cell. In some embodiments, the aptamer specifically binds an antigen on a cytotoxic or helper T cell. In some embodiments, the aptamer specifically binds an antigen on a macrophage. In some embodiments, the aptamer specifically binds an antigen on a natural killer cell. In some embodiments, the aptamer specifically binds an antigen on a neutrophil. In some embodiments, the aptamer specifically binds an antigen on an eosinophil. In some embodiments, the aptamer specifically binds an antigen on a γδ T cell. In some embodiments, the aptamer specifically binds an antigen on a natural killer T cell. In some embodiments, the aptamer specifically binds an antigen on an engineered immune cell. In some embodiments, the engineered immune cell is a CAR T cell, natural killer cell, natural killer T cell, or γδ T cell.

In some embodiments, the aptamer comprises DNA. In some embodiments, the aptamer comprises RNA. In some embodiments, the aptamer is single-stranded. In some embodiments, the aptamer is a selective immune cell binding-specific aptamer chosen from a random candidate library.

In some embodiments, the targeting moiety is an antibody or antigen-specific binding fragment. In some embodiments, the antibody or antigen-specific binding fragment thereof specifically binds a cancer antigen. In some embodiments, the targeting moiety is an aptamer. In some embodiments, the aptamer specifically binds a cancer antigen. In some embodiments, the aptamer comprises DNA. In some embodiments, the aptamer comprises RNA. In some embodiments, the aptamer is single-stranded. In some embodiments, the aptamer is a target cell-specific aptamer chosen from a random candidate library. In some embodiments, the aptamer is an anti-EGFR aptamer. In some embodiments, the anti-EGFR aptamer comprises any one of SEQ ID NOs: 95-164. In some embodiments, the aptamer binds to the cancer on the cancer cell with a K_{d} from 1 picomolar to 500 nanomolar. In some embodiments, the aptamer binds to the cancer with a K_{d} from 1 picomolar to 100 nanomolar.

In some embodiments, the targeting moiety comprises IL-2, IL-4, IL-6, α-MSH, transferrin, folic acid, EGF, TGF, PD1, IL-13, stem cell factor, insulin-like growth factor (IGF), or CD40. In some embodiments, the targeting moiety comprises a full-length sequence of IL-2, IL-4, IL-6, α-MSH, transferrin, folic acid, EGF, TGF, PD1, IL-13, stem cell factor, insulin-like growth factor (IGF), or CD40. In some embodiments, the targeting moiety comprises a truncated form, analog, variant, or derivative of IL-2, IL-4, IL-6, α-MSH, transferrin, folic acid, EGF, TGF, PD1, IL-13, stem cell factor, insulin-like growth factor (IGF), or CD40. In some embodiments, the targeting moiety binds a target on the cancer comprising IL-2 receptor, IL-4, IL-6, melanocyte stimulating hormone receptor (MSH receptor), transferrin receptor (TR), folate receptor 1 (FOLR), folate hydroxylase (FOLH1), EGF receptor, PD-L1, PD-L2, IL-13R, CXCR4, IGFR, or CD40L.

In some embodiments, one immune cell engaging domain comprises a VH domain and the other immune cell engaging domain comprises a VL domain. In some embodiments, the first immune cell binding partner is bound to the inert binding partner and separated from it by a cleavage site.

In some embodiments, the second immune cell binding partner is bound to the inert binding partner and separated from it by a cleavage site.

This application also describes an agent, wherein the first immune cell binding partner is bound to the inert binding partner and separated from it by a first cleavage site and the second immune cell binding partner is bound to the inert binding partner and separated from it by a second cleavage site.

In some embodiments, the first cleavage site and the second cleavage site are the same cleavage site. In some embodiments, the first cleavage site and the second cleavage site are different cleavage sites.

In some embodiments, at least one cleavage site is a protease cleavage site.

In some embodiments, at least one enzyme expressed by the cancer cells is a protease.

In some embodiments, at least one inert binding partner specifically binds the immune cell engaging domain. In some embodiments, at least one inert binding partner is a VH or VL domain.

In some embodiments, when the immune cell engaging domain is a VH domain, the inert binding partner is a VL domain, and when the immune cell engaging domain is VL domain, the inert binding partner is a VH domain.

This application also describes an agent for use in a two-component system for treating cancer comprising a a selective immune cell binding agent comprising: (a) a first component comprising a targeted immune cell binding agent comprising: (i) a targeting moiety capable of targeting the cancer; (ii) a first immune cell engaging domain capable of immune engaging activity when binding a second immune cell engaging domain, wherein the second immune cell engaging domain is not part of the first component; (b) a cleavage site separating the first immune cell engaging domain and the inert binding partner, wherein the cleavage site is: (i) cleaved by an enzyme expressed by the cancer cells; (ii) cleaved through a pH-sensitive cleavage reaction inside the cancer cell; (iii) cleaved by a complement-dependent cleavage reaction; or (iv) cleaved by a protease that is colocalized to the cancer cell by a targeting moiety that is the same or different from the targeting moiety in the agent, wherein cleavage of the cleavage site causes loss of the inert binding partner and allows for binding to the second immune cell engaging domain that is not part of the agent.

In some embodiments, the first component is covalently bound to the second component by a linker comprising a cleavage site.

In some embodiments, the cleavage site is a protease cleavage site.

In some embodiments, the protease cleavage site is cleavable in blood. In some embodiments, the protease cleavage site is a cleavage site for thrombin, neutrophil elastase, or furin.

In some embodiments, the protease cleavage site is cleavable by a tumor-associated protease. In some embodiments, the tumor-associated protease cleavage site comprises any one of SEQ ID NOs: 1-84.

This application also describes a set of nucleic acid molecules encoding the first and second component of the agent.

This application also describes a nucleic acid molecule encoding the selective immune cell binding agent.

This application also describes methods of treating cancer in a patient comprising administering the agent described herein.

In some embodiments, if the patient has regulatory T cells in the tumor, the selective immune cell binding agent does not target markers present on regulatory immune cells (including, but not limited to CD4 and CD25).

In some embodiments, the selective immune cell binding agent does not target markers present on TH17 cells. In some embodiments, the selective immune cell binding agent activates T cells that will target the tumor cells for lysis.

In some embodiments, if the patient has regulatory T cells in the tumor, the immune cell selection moiety targets CD8+ T cells by specifically binding CD8.

In some embodiments, if the patient has regulatory T cells in the tumor, the immune cell selection moiety targets CD8+ T cells and CD4+ T cells by specifically binding CXCR3.

In some embodiments, the cancer is any one of breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, liver cancer, leukemia, myeloma, nonHodgkin lymphoma, Hodgkin lymphoma, acute myeloid leukemia, acute lymphoblastic leukemia, chronic lymphoblastic leukemia, lymphoproliferative disorder, myelodysplastic disorder, myeloproliferative disease or premalignant disease.

This application also describes a method of targeting an immune response of a patient to cancer comprising administering an agent described herein to a patient.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1B provide a diagrammatic representation of the agent for treating cancer in a patient. As shown in FIG. 1A (timepoint 1), the agent is comprised of a first component comprising a targeted immune cell binding agent (ATTAC1) and a second component comprising a selective immune cell binding agent (ATTAC 2). ATTAC1 specifically binds to a cancer cell (circle and circular binding moiety) and ATTAC2 specifically binds to an immune cell (square and square binding moiety). ATTAC1 and ATTAC2 both comprise one half of an immune cell engaging domain capable of immune cell engaging activity (shown as bean shapes). Neither ATTAC1 nor ATTAC2 are capable of immune cell engaging activity unless they are bound to each other. Thus, by “targeted immune cell binding agent” we mean an agent that is capable of targeting to a cancer cell and that is capable of immune cell engaging activity when bound to the selective immune cell binding agent. Likewise, by a “selective immune cell binding agent” we mean an agent that is capable of selectively binding to a type of immune cell and that is capable of immune cell engaging activity when bound to the targeted immune cell binding agent. At least one and optionally both of the immune engaging domains are masked by an inert binding partner (here both are shown as masked). Until the at least one (or optionally both) inert binding domains are removed by cleavage of a cleavage site, the immune activity moiety (shown as a triangle) remains unengaged. The cleavage site separating each inert binding partner and immune cell engaging domain is shown as a rectangle. As shown in FIG. 1B (timepoint 2) enzymatic cleavage of the inert binding partner permits association of the first immune cell engaging domain and the second immune engaging domain to specifically activate the immune cell through binding of the immune cell engaging domain (here a VH-VL) to an antigen on the immune cell (shown at a triangle). This results in results in destruction of the cancer cell.

FIGS. 2A-2B show the logical control of the specificity of two-component structures, or T-cell engaging antibody circuits (TEACs), as discussed in WO 2017/087789 (FIG. 2A) compared to the current ATTAC structure (FIG. 2B) described herein. The TEAC employed a first component with both (i) a targeting moiety capable of targeting the cancer (“antigen 1”) and (ii) a cleavage site (“protease 1”) and a second component with (i) a targeting moiety capable of targeting the cancer (“antigen 2”) and (ii) an optional cleavage site (“protease 2”). The current ATTAC structure eliminates the specificity of the second component to the cancer (no longer includes a moiety targeting to antigen 2) and replaces it with an immune cell selection moiety capable of selectively targeting an immune cell (“immune cell marker”). In the ATTAC at least the first or second component comprises a cleavage site and here the cleavage site is shown on the first component and optional on the second component. The reverse configuration also applies.

FIGS. 3A-3C show T-cell activation by TEACs, showing that labeling T-cells with FITC-conjugated antibodies does not alter their ability to recognize the CD3 molecule on the tumor cell surface and become activated in response to it. T cells were labelled with different FITC-conjugated antibodies; target cells (MCF-7) were labelled with EpCAM V_{H} (SEQ ID NO: 166) and EpCAM V_{L} (SEQ ID NO: 167) TEAC components (20G6). Controls were labelled with BiTE (SEQ ID NO: 168). FIG. 3A shows IFN gamma release with the TEAC labelled tumor cells. FIG. 3B (CD4 T cells) and FIG. 3C (CD8 T cells) demonstrate T cell activation by CD69 flow cytometry staining using the mean fluorescence intensity (MFI) above background as readout. There was a strong T-cell response to EpCAM TEAC component pair when T cells were labeled with FITC conjugated antibodies. There was no blocking by bound antibodies. The TEACs activated both CD4 and CD8 cells and did not differentiate between them because both cell types express CD3. This control experiment shows that TEACs are not selective between CD4 and CD8 and that using an FITC model did not alter the expected results. The use of the FITC model does not prevent T cell activation. The results seen in FIG. 3A-C demonstrate the activation of all T cell subsets (CD4 and CD8) when there is a full anti-CD3 activating domain on the tumor cell.

FIGS. 4A-4C provide selective T-cell activation by ATTACs, using an experimental design where the tumor cells have only one ATTAC component and the T cells have the anti-FITC ATTAC component. T cells were PL labelled with different FITC-conjugated antibodies and then labelled with anti-FITC ATTAC component (CD3 V_{L} (20G6); SEQ ID NO: 165); target cells (MCF-7) labelled with EpCAM V_{H} ATTAC component (20G6; SEQ ID NO: 166). FIG. 4A shows IFN gamma release with the ATTAC labelled tumor cells. FIG. 4B (CD4 T cells) and FIG. 4C (CD8 T cells) demonstrate T cell activation by CD69 flow cytometry staining using the MFI above background as readout. There was a strong T cell response to the EpCAM ATTAC component/FITC ATTAC component pair when T cells were labelled with FITC-conjugated antibodies bound to CD8, CD52, and CXCR3. When using the anti-CD8 FITC-conjugated antibody, there was selective activation of CD8 T cells without activation of CD4 T cells (shown as an arrowin FIGS. 4B and 4C).

FIGS. 5A-5I show T cell expression of proteins on their surface and that only binding the ATTAC component to CD52, CD8 and CXCR3 (via FITC) allows T cell activation. A range of T cell antigens was tested, as shown in FIG. 5A (CD5); FIG. 5B (CD8); FIG. 5C (CD28); FIG. 5D (CD45RO); FIG. 5E (CD52); FIG. 5F (HLA-DR); FIG. 5G (CD19); FIG. 5H (CD278 (ICOS)); and FIG. 5I (CD279 (PD-1)).

FIGS. 6A-6F show CD4 T-cell activation by TEACs is not inhibited by FITC antibodies. T cells were labelled with different FITC-conjugated antibodies; target cells (MCF-7) labelled with anti-EpCAM V_{H} and V_{L} TEAC components (20G6). FIG. 6A presents interferon gamma release. Flow cytometry raw data is presented for unlabeled T cells (FIG. 6B) or with CD-19 labeling (FIG. 6C), CD52 labeling (FIG. 6D), or CD8 labeling (Hit8a, 6E). FIG. 6F collates the flow cytometry data for CD4 T cells. There was a strong T cell response to the EpCAM TEAC component pair when T cells were labelled with FITC-conjugated antibodies. There was no blocking by bound antibodies.

FIGS. 7A-7F show CD8 T-cell activation by TEACs is not inhibited by FITC antibodies. Paneling is as described for FIGS. 6A-6F. There was a strong T cell response to the EpCAM TEAC component pair when T cells were labelled with FITC-conjugated antibodies. There was no blocking by bound antibodies.

FIGS. 8A-8F show selective CD4 T-cell activation by ATTACs. Paneling is as described for FIGS. 6A-6F. There was a strong T cell response to the EpCAM ATTAC component/FITC ATTAC component pair when T cells were labelled with FITC-conjugated antibodies bound to CD8, CD52, or CXCR3. There was activation of CD4 T cells when using anti-CD52 or anti-CXCR3 FITC-conjugated antibodies.

FIGS. 9A-9F show selective CD8 T-cell activation by ATTACs. Paneling is as described for FIGS. 6A-6F. There was a strong T cell response to the EpCAM ATTAC component/FITC ATTAC component pair when T cells were labelled with FITC-conjugated antibodies bound to CD8, CD52, or CXCR3. There was activation of CD8 T cells when using anti-CD52, anti-CXCR3, or the four anti-CD8 FITC-conjugated antibodies.

FIGS. 10A and 10B show FACS results with EpCAM-expressing tumor cells. MDA-MB-231 cells over-expressing EpCAM were labelled with anti-EpCAM VH and VL to form a binding domain of the anti-CD8 ATTAC component is cleaved by enterokinase (protease). Controls for activation of T cells (FIG. 11 D) or T cells within PBMCs (FIG. 11C) included interferon release for T cells alone, in the presence of EpCAM BiTE (SEQ ID NO: 168; positive control), or when cultured with untreated target MDA-MB-231 cells (negative control). EpCAM VH refers to anti-EpCAM ATTAC1 (component targeting EpCAM cancer antigen and containing the anti-CD3 VH domain (SEQ ID NO: 166)). CD8 VL refers to anti-CD8 ATTAC2 (component targeting CD8 and containing the anti-CD3 VL domain (SEQ ID NO: 170)).

FIGS. 12A-12C show concentration dependence of ATTACs. MDA-MB-231 cells over-expressing EpCAM were labelled with increasing concentrations of EpCAM VH ATTAC component. T cells or healthy donor PBMCs were labelled with increasing concentrations of anti-CD8 VL ATTAC component (SEQ ID NO: 172). FIG. 12A shows results for cells co-cultured overnight and assayed for T cell activation by IFN gamma release. EpCAMx20G6-Vh refers to the anti-EpCAM and anti-CD3 VH ATTAC component, while CD8x20G6-VL refers to the anti-CD8 and anti-CD3 VL ATTAC component. The concentrations of both ATTAC components were not kept equal to determine if there was a dominant ATTAC component in the assay. The inert binding domain of the anti-CD8 ATTAC component was cleaved by enterokinase (protease). FIG. 12B shows results of increasing concentrations of both ATTAC components. Controls included interferon release from T cells in PBMCs cultured alone, in the presence of EpCAM BiTE (SEQ ID NO: 168; positive control), or when cultured with untreated target MDA-MB-231 cells (negative control) (FIG. 12C).

FIGS. 13A and 13B demonstrate activation of either CD4 or CD8 T cells using the ATTAC1 binding to the tumor cell and ATTAC2 binding to FITC conjugated antibodies bound to T cells in a mixed T-cell activation assay. PBMCs were labelled with either CD4-FITC, CD8-FITC, or CD19-FITC (negative control) and cultured with tumor cells bound by ATTAC1. Only CD4 T cells are activated when anti-CD4-FITC is bound to the T cells, and CD8 T cells are only activated when anti-CD8 FITC is bound to the T cells. This confirms the idea that binding of ATTAC2 to a subset of T cells activates only those T cells bound with ATTAC2 and not other T cell subsets that are not bound by ATTAC2.

### DESCRIPTION OF THE SEQUENCES

Table 1A provides a listing of certain sequences referenced herein. Table 1B provides a listing of certain construct sequences used herein.

TABLE 1A
Description of the Sequences and SEQ ID NOs

| Description | Sequence | # |
|---|---|---|
| ADAM28 cleavage site | KPAKFFRL | 1 |
| ADAM28 cleavage site | DPAKFFRL | 2 |
| ADAM28 cleavage site | KPMKFFRL | 3 |
| ADAM28 cleavage site | LPAKFFRL | 4 |
| ADAM28 cleavage site | LPMKFFRL | 5 |
| ADAM28 cleavage site | KPAMFFRL | 6 |
| ADAM28 cleavage site | YPAKFFRL | 7 |
| ADAM28 cleavage site | KWAKFFRL | 8 |
| ADAM28 cleavage site | DPMKFFRL | 9 |
| ADAM28 cleavage site | DPAMFFRL | 10 |
| ADAM28 cleavage site | DPMMFFRL | 11 |
| ADAM28 cleavage site | KMAMFFRL | 12 |
| ADAM28 cleavage site | KMAMFFIM | 13 |
| ADAM28 cleavage site | KPAMFFIM | 14 |
| ADAM28 cleavage site | LPAMFFRL | 15 |
| ADAM28 cleavage site | LPMMFFRL | 16 |
| ADAM28 cleavage site | LMAMFFRL | 17 |
| ADAM28 cleavage site | LMAMFFIM | 18 |
| ADAM28 cleavage site | LPAMFFIM | 19 |
| ADAM28 cleavage site | LPAMFFYM | 20 |
| ADAM28 cleavage site | KPMMFFRL | 21 |
| ADAM28 cleavage site | KPAKFFYM | 22 |
| ADAM28 cleavage site | KPAKFFIM | 23 |
| ADAM28 cleavage site | IPMKFFRL | 24 |
| ADAM28 cleavage site | IPAMFFRL | 25 |
| ADAM28 cleavage site | IPMMFFRL | 26 |
| ADAM28 cleavage site | IMAMFFRL | 27 |
| ADAM28 cleavage site | IMAMFFIM | 28 |
| ADAM28 cleavage site | IPAMFFIM | 29 |
| ADAM28 cleavage site | IPAMFFYM | 30 |
| cathepsin B cleavage site | FR | 31 |
| cathepsin B cleavage site | FK | 32 |
| cathepsin B cleavage site | VA | 33 |
| cathepsin B cleavage site | VR | 34 |
| cathepsin B cleavage site | V{Cit} | 35 |
| {Cit} = citrulline | | |
| cathepsin B cleavage site | HLVEALYL | 36 |
| cathepsin B cleavage site | SLLKSRMVPNFN | 37 |
| cathepsin B cleavage site | SLLIARRMPNFN | 38 |
| cathepsin B cleavage site | KKFA | 39 |
| cathepsin B cleavage site | AFKK | 40 |
| cathepsin B cleavage site | QQQ | 41 |
| cathepsin D cleavage site | PRSFFRLGK | 42 |
| cathepsin D cleavage site | SGVVIATVIVIT | 43 |
| cathepsin K cleavage site | GGP | 44 |
| MMP1 cleavage site | SLGPQGIWGQFN | 45 |
| MMP2 cleavage site | AIPVSLR | 46 |
| MMP2 cleavage site | SLPLGLWAPNFN | 47 |
| MMP2 cleavage site | HPVGLLAR | 48 |
| MMP2 cleavage site | GPLGVRGK | 49 |
| MMP2 cleavage site | GPLGLWAQ | 50 |
| MMP3 cleavage site | STAVIVSA | 51 |
| MMP7 cleavage site | GPLGLARK | 52 |
| MMP7 cleavage site | RPLALWRS | 53 |
| MMP7 cleavage site | SLRPLALWRSFN | 54 |
| MMP2/9 cleavage site | GILGVP | 55 |
| MMP2/9 cleavage site | GPLGIAGQ | 56 |
| MMP9 cleavage site | AVRWLLTA | 57 |
| MMP9 cleavage site | PLGLYAL | 58 |
| MMP9 cleavage site | GPQGIAGQR | 59 |
| MMP9 cleavage site | KPVSLSYR | 60 |
| MMP11 cleavage site | AAATSIAM | 61 |
| MMP11 cleavage site | AAGAMFLE | 62 |
| MMP13 cleavage site | GPQGLAGQRGIV | 63 |
| MMP14 cleavage site | PRHLR | 64 |
| MMP14 cleavage site | PQGLLGAPGILG | 65 |
| MMP14 cleavage site | PRSAKELR | 66 |
| PSA/KLK3 | HSSKLQ | 67 |
| PSA/KLK3 | SSKLQ | 68 |
| KLK4 | RQQR | 69 |
| TMPRSS2 | GGR | 70 |
| Legumain | AAN | 71 |
| ST14 (Matriptase) | QAR | 72 |
| C1s cleavage site | YLGRSYKV | 73 |
| C1s cleavage site | MQLGRX | 74 |
| MASP2 cleavage site | SLGRKIQI | 75 |
| C2a and Bb cleavage site | GLARSNLDE | 76 |
| uPa cleavage site | TYSRSRYL | 77 |
| uPa cleavage site | KKSPGRVVGGSV | 78 |
| uPa cleavage site | NSGRAVTY | 79 |
| uPa cleavage site | AFK | 80 |
| tissue-type plasminogen | GGSGQRGRKALE | 81 |
| activator (tPA) | | |
| ADAM10 | PRYEAYKMGK | 82 |
| ADAM12 | LAQAF | 83 |
| ADAM17 | EHADLLAVVAK | 84 |
| flexible amino acid linker | GGGGS | 85 |
| (may be presented in | | |
| repeating fashion) | | |
| flexible amino acid linker | GGGS | 86 |
| (may be presented in | | |
| repeating fashion) | | |
| flexible amino acid linker | GS | 87 |
| (may be presented in | | |
| repeating fashion) | | |
| flexible amino acid linker | GSGGS | 88 |
| (may be presented in | | |
| repeating fashion) | | |
| flexible amino acid linker | GGSG | 89 |
| (may be presented in | | |
| repeating fashion) | | |
| flexible amino acid linker | GGSGG | 90 |
| (may be presented in | | |
| repeating fashion) | | |
| flexible amino acid linker | GSGSG | 91 |
| (may be presented in | | |
| repeating fashion) | | |
| flexible amino acid linker | GSGGG | 92 |
| (may be presented in | | |
| repeating fashion) | | |
| flexible amino acid linker | GGGSG | 93 |
| (may be presented in | | |
| repeating fashion) | | |
| flexible amino acid linker | GSSSG | 94 |
| (may be presented in | | |
| repeating fashion) | | |
| Anti-EGFR aptamer | UGCCGCUAUAAUGCACGGAUUUAAUCGCCGU | 95 |
| (tight binder with K_{d} = | AGAAAAGCAUGUCAAAGCCG | |
| 2.4 nM) | | |
| Anti-EGFR aptamer | UGGCGCUAAAUAGCACGGAAAUAAUCGCCGU | 96 |
| | AGAAAAGCAUGUCAAAGCCG | |
| Anti-EGFR aptamer | UGCUAGUAUAUCGCACGGAUUUAAUCGCCGU | 97 |
| | AGAAAAGCAUGUCAAAGCCG | |
| Anti-EGFR aptamer | UGCCGCCAUAUCACACGGAUUUAAUCGCCGU | 98 |
| | AGAAAAGCAUGUCAAAGCCG | |
| Anti-EGFR aptamer | UUCCGCUGUAUAACACGGACUUAAUCGCCGU | 99 |
| | AGUAAAGCAUGUCAAAGCCG | |
| Anti-EGFR aptamer | UGUCGCUCUAUUGCACGGAUUUAAUCGCCGU | 100 |
| | AGAAAAGCAUGUCAAAGCCG | |
| Anti-EGFR aptamer | UGCUGCUUUAUCCCACAUAUUUUUUCCCCUC | 101 |
| | AUAACAAUAUUUCUCCCCCC | |
| Anti-EGFR aptamer | UGCNGCUAUAUCGCNCGUAUUUAAUCGCCGU | 102 |
| | AGAAAAGCAUGUCNANGCCG | |
| Anti-EGFR aptamer | UGCAAAGAAAACGCACGUAUUUAAUCGCCGU | 103 |
| | AGUAAAGCAUGUCAAAGCCG | |
| Anti-EGFR aptamer | UGCAUCACUAUCGAACCUAUUUAAUCCACCA | 104 |
| | AAAUAAUUGCAAGUCCAUACUC | |
| Anti-EGFR aptamer | UGCCNNAAUAACACACNUAUAUAAUCGCCGU | 105 |
| | ACAAAAUCAUGUCAAANCCG | |
| Anti-EGFR aptamer | UGCAGCUGUAUUGCACGUAUUUAAUCGCCGU | 106 |
| | AGAAAAGCAUGUCAAAGCCG | |
| Anti-EGFR aptamer | UUCCGAUAAUCCCGCGUACUAAAUCACCAUA | 107 |
| | GUCAACAAUUUCCAACCUC | |
| Anti-EGFR aptamer | UCCACUAUAUCACACGUAUUUAAUCGCCGUA | 108 |
| | GAAAAGCAUGUCAAAGCCG | |
| Anti-EGFR aptamer | UCCCUCAACCUCGCUACUAUUUAAUCGCCGU | 109 |
| | AGAAAAGCAUGUCAAAGCCU | |
| Anti-EGFR aptamer | UGCCGCUAUAUCACACGAAUUUAAUCGCCGU | 110 |
| | AGAAAAGCAUGUCAAAGCCG | |
| Anti-EGFR aptamer | AGCCCCUAGAACACACGGAUUUAAUCGCCGU | 111 |
| | AGAAAAGCAUGUCAAAGCCG | |
| Anti-EGFR aptamer | UGCCAAUAUAUAACACGGAAUUAAUCGCCGU | 112 |
| | AGAAAAGCAUGUCAAAGCCG | |
| Anti-EGFR aptamer | UGCCGCUAUAGCGCACGGAUUUAAUCGCCGU | 113 |
| | AGAAAAGCAUGUCAAAGCCG | |
| Anti-EGFR aptamer | UGCAGAUAUAUGUCACUCAUUAAUCCCCGUA | 114 |
| | UAAAAACAUAACUAAGCUC | |
| Anti-EGFR aptamer | UGUAGCUGUAUUGCACACAUUAAAUCGCCGU | 115 |
| | AGUAAAGCAUGUCAAAGCCG | |
| Anti-EGFR aptamer | UACCAAUAUAUCGCCACACAUAAUCGCCGUA | 116 |
| | GAAAAGCAUGUCAAAGCCG | |
| Anti-EGFR aptamer | UGCCGCUAUGCCCACGGAAUUUAAUCGCCGU | 117 |
| | AGAAAAACAUGUCAAAGUCG | |
| Anti-EGFR aptamer | UGCCGCUAUUUAGCACGGAUUAAAUCGCCGU | 118 |
| | AGAAAAGCAUGUCAAAGCCG | |
| Anti-EGFR aptamer | UGCCGCUAUUUAGCACGGAUUAAAUCGCCGU | 119 |
| | AGAAAAGCAUGUCNAAGCCG | |
| Anti-EGFR aptamer | UGUAGUAAUAUGACACGGAUUUAAUCGCCGU | 120 |
| | AGAAAAGCANGUCAAAGCCU | |
| Anti-EGFR aptamer | UGUCGCCAUUACGCACGGAUUUAAUCGCCGU | 121 |
| | AGAAAAGCAUGUCAAAGCCG | |
| Anti-EGFR aptamer | UGCCCCCAAACUACACAAAUUUAAUCGCCGU | 122 |
| | AUAAAAGCAUGUCAAAGCCG | |
| Anti-EGFR aptamer | UGCACUAUCUCACACGUACUAAUCGCCGUAU | 123 |
| | AAAAGCAUGUCAAAGCCG | |
| Anti-EGFR aptamer | UGUCGCAAUAAUACACUAAUUUAAUCGCCGU | 124 |
| | AGAAAAGCAUGUCAAAGCCG | |
| Anti-EGFR aptamer | UGCAACAAUAUAGCACGUAUUUAAUCGCCGU | 125 |
| | AGUAAAGCAUGUCAAAGG | |
| Anti-EGFR aptamer | CUACCACAAAUCCCACAUAUUUAAUCUCCCA | 126 |
| | AUCAAAUCUUGUCCAUUCCC | |
| Anti-EGFR aptamer | UGCCCUAAACUCACACGGAUAUAAUCGCCGU | 127 |
| | AGAAAAGCAUGUCAAAGCCG | |
| Anti-EGFR aptamer | UUGUCGUAUGUCACACGUAUUAAAUCGCCGU | 128 |
| | AUAAAAGCAUGUCAAAGCCG | |
| Anti-EGFR aptamer | UUCCGCUAUAACACACGGAGAAAAUCGCCGU | 129 |
| | AGUAAAGCAUGUCAAAGCCG | |
| Anti-EGFR aptamer | UGCCGAUAUAACGCACGGAUAUAAUCGCCGU | 130 |
| | AGAAAAGCAUGUCAAAGCCG | |
| Anti-EGFR aptamer | UGCCAUUAUACAGCACGGAUUUAAUCGCCGU | 131 |
| | AGAAAAGCAUGUCAAAGCCG | |
| Anti-EGFR aptamer | UCCAGAAAUAUGCACACAUUUAAUCGCCGUA | 132 |
| | GAAAAGCAUGUCAAAGCCG | |
| Anti-EGFR aptamer | UCCGCUAAACAACACGGAUACAAUCGCCGUA | 133 |
| | GAAAAGCAUGUCCAAGCCG | |
| Anti-EGFR aptamer | UGCACUAUCUCACACGUACUAAUCGCCGUAU | 134 |
| | AAAAGCAUGUCAAANNNG | |
| Anti-EGFR aptamer | AUNGCNANNNUACACGUAUUNAAUCGCCGUA | 135 |
| | GAAAAGCAUGUCANAGCCG | |
| Anti-EGFR aptamer | UGCUGCUAUAUUGCAAUUUUUUAAACUAAGU | 136 |
| | AGAAAACCAUGUACAAGUCG | |
| Anti-EGFR aptamer | UGUCGCCAUAUUGCACGGAUUUAAUCGCCGU | 137 |
| | AGAAAAGCAUGUCCAAGCCG | |
| Anti-EGFR aptamer | UGCCGUUAUAACCCACGGAAUUUAACCUCCG | 138 |
| | UAGAAAAGCAUGUCAAAGCCG | |
| Anti-EGFR aptamer | UGUGAAUAUAUAUCACGGAUUUAAUCGCCGU | 139 |
| | AUAAAAGCNAUGUCAAAGCCG | |
| Anti-EGFR aptamer | UGCCGAUAUNNANCACGGAUUUAAUCGCCGU | 140 |
| | AGAAAAGCAUGUCCAAGCCG | |
| Anti-EGFR aptamer | UGUCACUAAAUUGCACGUAUAUAAUCGCCGU | 141 |
| | AGUAAGCAUGUCAAAGCCG | |
| Anti-EGFR aptamer | UGCAACCAUAAAGCACGUAAUAAAUCGCCGU | 142 |
| | AUAUAAGCAUGUCaAAGCCG | |
| Anti-EGFR aptamer | UGCCGCUAUAUAGCACGUAUUAAUCGCCGUA | 143 |
| | GUAAAGCAUGUCaAAGCCG | |
| Anti-EGFR aptamer | UGCCGCUAUAGCACACGGAAUUUAAUCGCCG | 144 |
| | UAGUAAAGCAUGUCAAAGCCG | |
| Anti-EGFR aptamer | UGCAGGUAUAUAACNCGGAUUUAAUCGCCGU | 145 |
| | AGAAAAGCAUGUCNAAGCCG | |
| Anti-EGFR aptamer | UGCUCCUAUAACACACGGAUUUAAUCGCCGU | 146 |
| | AGAAAAGCAUGUCCAAGCCG | |
| Anti-EGFR aptamer | UGCCCGUAAUUGCACGGAUUUAAUCGCCGUA | 147 |
| | GAAAAGCAUGUCCAAGCCGG | |
| Anti-EGFR aptamer | ACUCCCUAUAUNGCAACUACAUAAUCGCCGU | 148 |
| | AAAUAAGCAUGUNCAAGCCG | |
| Anti-EGFR aptamer | UGAAGCUAGAUCACACUAAAUUAAUCGCCGU | 149 |
| | AGAAAAGCAUGUCAAAAAAGCCG | |
| Anti-EGFR aptamer | UGACUCUUUAUCCCCCGUACAUUAUUcACCG | 150 |
| | AACCAAAGCAUUACCAUCCCC | |
| Anti-EGFR aptamer | UGACGCCCUAACACACGUAUAUAAUCGCCGU | 151 |
| | AGAAAAGCAUGUCAAAGCCG | |
| Anti-EGFR aptamer | UGUCGCAAAAUAGCACGUAUUUAAUCGCCGU | 152 |
| | AGAAAAGCAUGUCCAAGCCG | |
| Anti-EGFR aptamer | UGAGUGUAUAAUUCACGUAUUUAAUCGCCGU | 153 |
| | AGAAAAGCAUGUCAAAGCCG | |
| Anti-EGFR aptamer | UGCUACUAUAUCGUAGGUAACUAAUCGCCCU | 154 |
| | ACAAACUCACUCUAAAACCG | |
| Anti-EGFR aptamer | UUACGCUAUAUCACACGGAAUUUUAAUCGCC | 155 |
| | GUAGAAAAGCAUGUCCAAGCCG | |
| Anti-EGFR aptamer | CCCAUCUGUACUACAGGAAUUUAAUCGCCGU | 156 |
| | AGAAAAGCAUGUCCAAGCCG | |
| Anti-EGFR aptamer | UGCCCAUAAAUAGCACGGAUUUAAUCGCCGU | 157 |
| | AGAAAAGCAUGUCCAAGCCG | |
| Anti-EGFR aptamer | UGCCGCAAUAACAUACACAUAUAAUCGCCGU | 158 |
| | AGAAAAGCAUGUCAAAGCCG | |
| Anti-EGFR aptamer | UGCAACUAUAUCGCACGUAUGUAAUCGCCGU | 159 |
| | AGAAAAAGCAUGUCAAAGCC | |
| Anti-EGFR aptamer | UUCCGCUAUAUAGCACGGAAUUAAUCGCCGU | 160 |
| | AGAAAAGCAUGUCCAAGCCG | |
| Anti-EGFR aptamer | UUCCGCUAAGUCACACGAAAUUAAUCGCCGU | 161 |
| | AGAAAAGCAUGUCCAAGCCG | |
| Anti-EGFR aptamer | UGUAGCAAUAUCACACGUAAUUAAUCGCCGU | 162 |
| | AUAUAAGCAUGUCAAAGCCG | |
| Anti-EGFR aptamer | UGCCGUUAUAUAUCACGGAUUUAAUCGCCGU | 163 |
| | AGAAAAGCAUGUCCAAGCCG | |
| Anti-EGFR aptamer | UAACACAUAUAUCAAGUAACUUAUCUCCUUA | 164 |
| | GUAACCAUCUCCAAGCCG | |

TABLE 1B
Description of Constructs and SEQ ID NOs

| Description | Sequence | # |
|---|---|---|
| Anti-FITC-CD3 VL | DVVMTQTPLSLPVSLGDQASISCRSSQSLVHSNG | 165 |
| ATTAC/TEAC | NTYLRWYLQKPGQSPKVLIYKVSNRVSGVPDRF | |
| component (Anti- | SGSGSGTDFTLKINRVEAEDLGVYFCSQSTHVPW | |
| Fluorescein scFv with | TFGGGTKLEIKSSADDAKKDAAKKDDAKKDDA | |
| linker between VL-VH- | KKDGGVKLDETGGGLVQPGGAMKLSCVTSGFT | |
| 1xG4S connector-anti- | FGHYWMNWVRQSPEKGLEWVAQFRNKPYNYE | |
| CD3e VL (20G6)- | TYYSDSVKGRFTISRDDSKSSVYLQMNNLRVED | |
| MMP2 cleavage | TGIYYCTGASYGMEYLGQGTSVTVSSGGGGSDI | |
| sequence-Ig VH domain- | VMTQTPLSLSVTPGQPASISCKSSQSLVHNNGNT | |
| His tag) | YLSWYLQKPGQSPQSLIYKVSNRFSGVPDRFSGS | |
| | GSGTDFTLKISRVEAEDVGVYYCGQGTQYPFTF | |
| | GSGTKVEIKGEGTSTGSGAIPVSLRGSGGSGGA | |
| | DQVQLVESGGGVVQPGRSLRLSCAASGFTFSSY | |
| | GIVIEIWVRQAPGKQLEWVAQISFDGSNKYYADS | |
| | VKGRFTISRDDSKNTLYLQMNSLRAEDTAVYYC | |
| | ASERGHYYDSSAFDYWGQGTLVTVSS [symbol] * | |
| Anti-EpCAM-CD3 VH | ELVMTQSPSSLTVTAGEKVTMSCKSSQSLLNSG | 166 |
| ATTAC/TEAC | NQKNYLTWYQQKPGQPPKLLIYWASTRESGVPD | |
| component (Anti- | RFTGSGSGTDFTLTISSVQAEDLAVYYCQNDYSY | |
| EpCAM scFv with | PLTFGAGTKLEIKGGGGSGGGGSGGGGSEVQL | |
| 3xG4S linker between | LEQSGAELVRPGTSVKISCKASGYAFTNYWLGW | |
| VH-VL-1xG4S | VKQRPGHGLEWIGDIFPGSGNIHYNEKFKGKATL | |
| connector-anti-CD3e | TADKSSSTAYMQLSSLTFEDSAVYFCARLRNWD | |
| VH (20G6)-MMP2 | EPMDYWGQGTTVTVSSGGGGSQVQLVESGGG | |
| cleavage sequence-Ig | VVQPGRSLRLSCAASGFTFTKAWMHWVRQAPG | |
| VL domain-His tag) | KQLEWVAQIKDKSNSYATYYADSVKGRFTISRD | |
| | DSKNTLYLQMNSLRAEDTAVYYCRGVYYALSP | |
| | FDYWGQGTLVTVSSGEGTSTGSGAIPVSLRGSG | |
| | GSGGADDIVMTQTPLSLSVTPGQPASISCKSSQSI | |
| | VHSSGNTYLSWYLQKPGQSPQLLIYKVSNRFSG | |
| | VPDRFSGSGSGTDFTLKISRVEAEDVGVYYCGQ | |
| | GSHVGPTFGSGTKVEIK [symbol] * | |
| Anti-EpCAM-CD3 VL | EVQLLEQSGAELVRPGTSVKISCKASGYAFTNY | 167 |
| ATTAC/TEAC | WLGWVKQRPGHGLEWIGDIFPGSGNIHYNEKFK | |
| component (Anti- | GKATLTADKSSSTAYMQLSSLTFEDSAVYFCAR | |
| EpCAM scFv with | LRNWDEPMDYWGQGTTVTVSSGGGGSGGGGS | |
| 3xG4S linker between | GGGGSELVMTQSPSSLTVTAGEKVTMSCKSSQS | |
| VH-VL-1xG4S | LLNSGNQKNYLTWYQQKPGQPPKLLIYWASTRE | |
| connector-anti-CD3e | SGVPDRFTGSGSGTDFTLTISSVQAEDLAVYYCQ | |
| VL (20G6)-MMP2 | NDYSYPLTFGAGTKLEIKGGGGSDIVMTQTPLSL | |
| cleavage sequence-Ig | SVTPGQPASISCKSSQSLVHNNGNTYLSWYLQKP | |
| VH domain-His tag) | GQSPQSLIYKVSNRFSGVPDRFSGSGSGTDFTLKI | |
| | SRVEAEDVGVYYCGQGTQYPFTFGSGTKVEIKG | |
| | EGTSTGSGAIPVSLRGSGGSGGADQVQLVESGG | |
| | GVVQPGRSLRLSCAASGFTFSSYGMHWVRQAPG | |
| | KQLEWVAQISFDGSNKYYADSVKGRFTISRDDS | |
| | KNTLYLQMNSLRAEDTAVYYCASERGHYYDSS | |
| | AFDYWGQGTLVTVSS [symbol] * | |
| Anti-EpCAM-CD3 scFv | ELVMTQSPSSLTVTAGEKVTMSCKSSQSLLNSG | 168 |
| (20G6) BiTE construct | NQKNYLTWYQQKPGQPPKLLIYWASTRESGVPD | |
| (anti-EpCAM scFv with | RFTGSGSGTDFTLTISSVQAEDLAVYYCQNDYSY | |
| 3xG4S linker between | PLTFGAGTKLEIKGGGGSGGGGSGGGGSEVQL | |
| VH and VL-1xG4S | LEQSGAELVRPGTSVKISCKASGYAFTNYWLGW | |
| connector-anti-CD3 | VKQRPGHGLEWIGDIFPGSGNIHYNEKFKGKATL | |
| scFv with linker between | TADKSSSTAYMQLSSLTFEDSAVYFCARLRNWD | |
| VH and VL-His Tag) | EPMDYWGQGTTVTVSSGGGGSDIVMTQTPLSLS | |
| | VTPGQPASISCKSSQSLVHNNGNTYLSWYLQKP | |
| | GQSPQSLIYKVSNRFSGVPDRFSGSGSGTDFTLKI | |
| | SRVEAEDVGVYYCGQGTQYPFTFGSGTKVEIKG | |
| | EGTSTGSGGSGGSGGADQVQLVESGGGVVQPG | |
| | RSLRLSCAASGFTFTKAWMIHWVRQAPGKQLEW | |
| | VAQIKDKSNSYATYYADSVKGRFTISRDDSKNT | |
| | LYLQMNSLRAEDTAVYYCRGVYYALSPFDYWG | |
| | QGTLVTVSS [symbol] * | |
| Anti-CD8-CD3 VL | QVQLQESGGGLVQPGGSLRLSCAASGFTFDDYA | 169 |
| ATTAC component | MSWVRQVPGKGLEWVSTINWNGGSAEYAEPVK | |
| (Anti-CD8 VHH-1xG4S | GRFTISRDNAKNTVYLQMNSLKLEDTAVYYCAK | |
| connector-anti-CD3e | DADLVWYNLRTGQGTQVTVSSAAAYPYDVPDY | |
| VL (20G6)-MMP2 | GSGGGGSDIVMTQTPLSLSVTPGQPASISCKSSQ | |
| cleavage sequence-Ig | SLVHNNGNTYLSWYLQKPGQSPQSLIYKVSNRF | |
| VH domain-His tag) | SGVPDRFSGSGSGTDFTLKISRVEAEDVGVYYCG | |
| (CD8 targeting VHH | QGTQYPFTFGSGTKVEIKGEGTSTGSGAIPVSLR | |
| domain based upon | GSGGSGGADQVQLVESGGGVVQPGRSLRLSCA | |
| WO_2017_134306 SEQ | ASGFTFSSYGMHWVRQAPGKQLEWVAQISFDGS | |
| ID NO: 20) | NKYYADSVKGRFTISRDDSKNTLYLQMNSLRAE | |
| | DTAVYYCASERGHYYDSSAFDYWGQGTLVTVS | |
| | S [symbol] * | |
| Anti-CD8-CD3 VL | EVQLQQSGAELVKPGASVKLSCTASGFNIKDTYI | 170 |
| ATTAC component | HFVRQRPEQGLEWIGRIDPANDNTLYASKFQGK | |
| (Anti-CD8 scFv with | ATITADTSSNTAYMHLCSLTSGDTAVYYCGRGY | |
| linker between VL-VH- | GYYVFDHWGQGTTLTVSSGGGGSGGGGSGGG | |
| 1xG4S connector-anti- | GSDVQINQSPSFLAASPGETITINCRTSRSISQYLA | |
| CD3e VL (20G6)- | WYQEKPGKTNKLLIYSGSTLQSGIPSRFSGSGSG | |
| MMP2 cleavage | TDFTLTISGLEPEDFAMYYCQQHNENPLTFGAGT | |
| sequence-Ig VH domain- | KLELKGGGGSDIVMTQTPLSLSVTPGQPASISCK | |
| His tag) | SSQSLVHNNGNTYLSWYLQKPGQSPQSLIYKVS | |
| (CD8 targeting scFv | NRFSGVPDRFSGSGSGTDFTLKISRVEAEDVGVY | |
| domain based upon | YCGQGTQYPFTFGSGTKVEIKGEGTSTGSGAIPV | |
| OKT8 antibody) | SLRGSGGSGGADQVQLVESGGGVVQPGRSLRLS | |
| | CAASGFTFSSYGMHWVRQAPGKQLEWVAQISF | |
| | DGSNKYYADSVKGRFTISRDDSKNTLYLQMNSL | |
| | RAEDTAVYYCASERGHYYDSSAFDYWGQGTLV | |
| | TVSS [symbol] * | |
| Anti-CD4-CD3 VL | QVQLQQSGPEVVKPGASVKMSCKASGYTFTSYV | 171 |
| ATTAC component | IHWVRQKPGQGLDWIGYINPYNDGTDYDEKFK | |
| (Anti-CD4 scFv with | GKATLTSDTSTSTAYMELSSLRSEDTAVYYCAR | |
| linker between VL-VH- | EKDNYATGAWFAYWGQGTLVTVSSGGGGSGG | |
| 1xG4S connector-anti- | GGSGGGGSDIVMTQSPDSLAVSLGERVTMNCK | |
| CD3e VL (20G6)- | SSQSLLYSTNQKNYLAWYQQKPGQSPKLLIYWA | |
| MMP2 cleavage | STRESGVPDRFSGSGSGTDFTLTISSVQAEDVAV | |
| sequence-Ig VH domain- | YYCQQYYSYRTFGGGTKLEIKGGGGSDIVMTQT | |
| His tag) | PLSLSVTPGQPASISCKSSQSLVHNNGNTYLSWY | |
| (CD4 targeting scFv | LQKPGQSPQSLIYKVSNRFSGVPDRFSGSGSGTD | |
| domain based upon | FTLKISRVEAEDVGVYYCGQGTQYPFTFGSGTK | |
| Ibalizumab antibody) | VEIKGEGTSTGSGAIPVSLRGSGGSGGADQVQL | |
| | VESGGGVVQPGRSLRLSCAASGFTFSSYGMHWV | |
| | RQAPGKQLEWVAQISFDGSNKYYADSVKGRFTI | |
| | SRDDSKNTLYLQMNSLRAEDTAVYYCASERGH | |
| | YYDSSAFDYWGQGTLVTVSS [symbol] * | |
| Anti-CD8-CD3 VL | QVQLQESGGGLVQAGGSLRLSCAASGFTFDDYA | 172 |
| ATTAC component | IGWFRQAPGKEREGVSCIRVSDGSTYYADPVKG | |
| (Anti-CD8 VHH-6xG4S | RFTISSDNAKNTVYLQMNSLKPEDAAVYYCAAG | |
| connector-anti-CD3e | SLYTCVQSIVWPARPYYDMDYWGKGTQVTVSS | |
| VL (20G6)- | AAAYPYDVPDYGSGGGGSGGGGSGGGGSGG | |
| Enterokinase cleavage | GGSGGGGSGGGGSDIVMTQTPLSLSVTPGQPAS | |
| sequence-Ig VH domain- | ISCKSSQSLVHNNGNTYLSWYLQKPGQSPQSLIY | |
| His tag) | KVSNRFSGVPDRFSGSGSGTDFTLKISRVEAEDV | |
| (CD8 targeting VHH | GVYYCGQGTQYPFTFGSGTKVEIKGEGTSTGSG | |
| domain based upon | GGGGSGGGGSDDDDKGGGGSGGGGSGSGGSGG | |
| WO_2017_134306 SEQ | ADQVQLVQSGAEVKKPGASVKVSCKASGYTFTS | |
| ID NO: 21) | YYIHWVRQAPGQGLEWIGCIYPGNVNTNYNEKF | |
| | KDRATLTVDTSISTAYMELSRLRSDDTAVYFCTR | |
| | SHYGLDWNFDVWGQGTTVTVSSGSHHHHHH* | |

### DESCRIPTION OF THE EMBODIMENTS

### I. ATTACs

The term ATTAC refers to a antibody tumor-targeting assembly complex. By using the word complex, the application refers to the need to have both a first component and a second component to make a complete functional molecule (i.e., the “complex”). The term complex also refers to the Boolean operator logic based upon (i) antigen expression on cancer cells, (ii) protease locations, and (iii) immune cell markers on desired immune cells. By applying logic gating, we obviate many of the current challenges with T-cell engaging antibodies.

ATTACs refer to using one ATTAC component that binds to a cancer antigen and one ATTAC component that does not bind to a cancer antigen, but instead selectively targets an immune cell. Thus, the ATTAC components do not have a parallel configuration (as in prior agents where both members of the ATTAC pair bound to cancer antigens), but instead have a trans configuration.

In an ATTAC component or pair, a first component comprising (a) a targeted immune cell binding agent comprises:
i. a targeting moiety capable of targeting the cancer;
ii. a first immune cell engaging domain capable of immune cell engaging activity when binding a second immune cell engaging domain, wherein the second immune cell engaging domain is not part of the first component;
and (b) a second component comprising a selective immune cell binding agent comprises:
i. an immune cell selection moiety capable of selectively targeting an immune cell;
ii. a second immune cell engaging domain capable of immune cell engaging activity when binding the first immune cell engaging domain, wherein the first and second immune cell engaging domains are capable of binding when neither is bound to an inert binding partner.

At least one of the first immune cell engaging domain or the second immune cell engaging domain is bound to an inert binding partner such at the first and second immune cell engaging domains are not bound to each other unless the inert binding partner is removed. The inert binding partner, when present, is bound to the immune cell engaging domain by a cleavage site separating the inert binding partner and the immune cell engaging domain to which it binds, wherein the cleavage site is:
a. cleaved by an enzyme expressed by the cancer cells;
b. cleaved through a pH-sensitive cleavage reaction inside the cancer cell;
c. cleaved by a complement-dependent cleavage reaction; or
d. cleaved by a protease that is colocalized to the cancer cell by a targeting moiety that is the same or different from the targeting moiety in the agent.

A. Single Polypeptide Chain or Two Components

In some embodiments, the first component is covalently bound to the second component. In some embodiments, the first component is not covalently bound to the second component.

In some embodiments, the ATTAC is comprised of two separate components.

In other words, the ATTAC can be comprised of a first and second component that are separate polypeptides.

In some components, the ATTAC is comprised of a single polypeptide chain. In some embodiments, the first and second components are contained within a single amino acid sequence.

When the ATTAC is comprised of a single polypeptide chain, the first and second components may be separated by a linker. In some embodiments, this linker covalently binds the first and second components. In some embodiments, this linker comprises a cleavable linker. In some embodiments, the cleavable linker between the first and second components comprises a protease cleavage site.

In some embodiments, a cleavage site comprised within a linker covalently binding a first component and the second component is a protease cleavage site. SEQ ID NOs: 1-84 list some exemplary protease cleavage sites that may be used, but the invention is not limited to this set of proteases cleavage sites and other protease cleavage sites may be employed.

In some embodiments, a cleavage site comprised within a linker covalently binding a first component and the second component is a tumor-associated protease cleavage site. A tumor associated protease is one that is associated with a tumor. In some embodiments, a tumor-associated protease has higher expression in the tumor versus other regions of the body. Table 3A provides examples of tumor-associated proteases, although any protease with expression in a tumor may be used to select a tumor-associated protease cleavage site for the invention.

In some embodiments, a cleavage site comprised within a linker covalently binding a first component and the second component is a cleavage site for a protease found in the blood. Exemplary proteases found in the blood include thrombin, neutrophil elastase, and furin.

B. Immune Cell Selection Moiety

In some embodiments, an ATTAC comprises an immune cell selection moiety specific for a particular immune cell. In some embodiments, the immune cell selection moiety is specific for CD8+ T cells, CD4+ T cells, natural killer (NK) cells, macrophages, neutrophils, eosinophils, basophils, γδ T cells, natural killer T cells (NKT cells), or engineered immune cells. Engineered immune cells refers to immune cells with engineered receptors with new specificity. Examples of engineered immune cells include chimeric antigen receptor (CAR) T cells, NK, NKT, or γδ T cells.

In some embodiments, the immune cell selection moiety targets an immune cell marker that is not a tumor antigen. In some embodiments, the immune cell selection moiety allows targeting of an ATTAC to an immune cell, wherein the immune cell is not a cancer cell. In some embodiments, the immune cell selection moiety does not target the ATTAC to a lymphoma, myeloma, or leukemia. In some embodiments, the ATTAC targets a solid tumor (in other words any tumor not of an immune cell).

In some embodiments, the immune cell selection moiety does not specifically bind regulatory T cells. In some embodiments, the immune cell selection moiety does not specifically bind TH17 cells. In some embodiments, the selective immune cell binding agent does not target markers present on regulatory immune cells (including, but not limited to CD4 and CD25).

Table 2 lists some representative immune cell selection moieties for different desired immune cells.

TABLE 2
Immune Cell Selection Moiety

| | | Immune | |
|---|---|---|---|
| Desired | Immune | Cell | |
| Immune | Cell | Selection | |
| Cell | Marker | Moiety | Citations for Representative Species |
| CD8+ T | CD8 | Antibodies | El Menshawy N et al. CD58; Leucocyte Function Adhesion-3 (LFA-3) Could Be Used as a |
| Cells | | or antigen | Differentiating Marker between Immune and Non-Immune Thyroid Disorders. |
| | | binding | Comparative Clinical Pathology 27.3: 721-727 (2018). |
| | | fragments | Guo Y et al. Immune checkpoint inhibitor PD-1 pathway is down-regulated in synovium at |
| | | thereof to | various stages of rheumatoid arthritis disease progression. Heymann D, ed. PLoS ONE. |
| | | CD8 | 2018; 13(2): e0192704. |
| | | | Tavaré R, Escuin-Ordinas H, Mok S, et al. An effective immuno-PET imaging method to |
| | | | monitor CD8-dependent responses to immunotherapy. Cancer Research. 76(1): 73-82 |
| | | | (2016). |
| | | | Darmochwal-Kolarz D et al. CD3⁺CD8⁺ Lymphocytes Are More Susceptible for Apoptosis |
| | | | in the First Trimester of Normal Human Pregnancy. Journal of Immunology Research. |
| | | | 2014: 670524 (2014). |
| | | | Chen G et al. Cigarette Smoke Disturbs the Survival of CD8+ Tc/Tregs Partially through |
| | | | Muscarinic Receptors-Dependent Mechanisms in Chronic Obstructive Pulmonary Disease. |
| | | | Su Y, ed. PLoS ONE. 11(1): e0147232 (2016). |
| | | | Brazowski E et al. FOXP3 expression in duodenal mucosa in pediatric patients with celiac |
| | | | disease. Pathobiology. 77(6): 328-34 (2010). |
| | | | Clement M et al. Anti-CD8 antibodies can trigger CD8+ T cell effector function in the |
| | | | absence of TCR engagement and improve peptide-MHCI tetramer staining. J Immunol. |
| | | | 187(2): 654-63 (2011). |
| | | Aptamers | Wang CW et al. A new nucleic acid-based agent inhibits cytotoxic T lymphocyte-mediated |
| | | to CD8 | immune disorders. J Allergy Clin Immunol. 132(3): 713-722 (2013). |
| | CXCR3 | Antibodies | Robert R et al. A fully humanized IgG-like bispecific antibody for effective dual targeting |
| | | or antigen | of CXCR3 and CCR6. PLoS One. 12(9): e0184278 (2017). |
| | | binding | Lintermans L L, Rutgers A, Stegeman C A, Heeringa P, Abdulahad W H. Chemokine |
| | | fragments | receptor co-expression reveals aberrantly distributed TH effector memory cells in GPA |
| | | thereof to | patients. Arthritis Research & Therapy. 19: 136 (2017). |
| | | CXCR3 | Rojas-Dotor S et al. Expression of resistin, CXCR3, IP-10, CCR5 and MIP-1α in obese |
| | | | patients with different severity of asthma. |
| | | | Biol Res. 46(1): 13-20 (2013). |
| | | | Agostini C et al. Involvement of the IP-10 Chemokine in Sarcoid Granulomatous |
| | | | Reactions. J Immunol. 161 (11) 6413-6420 (1998). |
| | | | Jiskra J et al. CXCR3, CCR5, and CRTH2 Chemokine Receptor Expression in |
| | | | Lymphocytes Infiltrating Thyroid Nodules with Coincident Hashimoto's Thyroiditis |
| | | | Obtained by Fine Needle Aspiration Biopsy. J Immunol Res. 2016: 2743614 (2016). |
| | | | Lübbers J et al. Changes in peripheral blood lymphocyte subsets during arthritis |
| | | | development in arthralgia patients. Arthritis Research & Therapy. 18: 205 (2016). |
| CD4+ T | CD4 | Antibodies | De Graav GN et al. Follicular T helper cells and humoral reactivity in kidney transplant |
| Cells | | or antigen | patients. Clin Exp Immunol. 180(2): 329-340 (2015). |
| | | binding | Duluc D et al. Induction and activation of human Th17 by targeting antigens to dendritic |
| | | fragments | cells via Dectin-1. J Immunol 192(12): 5776-5788 (2014). |
| | | thereof to | Flamar, Anne-Laure et al. “Targeting Concatenated HIV Antigens to Human CD40 |
| | | CD4 | Expands a Broad Repertoire of Multifunctional CD4+ and CD8+ T Cells.” AIDS. 27: 13 |
| | | | (2013). |
| | | | Almanzar G et al. Autoreactive HSP60 epitope-specific T-cells in early human |
| | | | atherosclerotic lesions. J Autoimmun. 39(4): 441-50 (2012). |
| | | | Babaei A et al. Production of a recombinant anti-human CD4 single-chain variable- |
| | | | fragment antibody using phage display technology and its expression in Escherichia coli. J |
| | | | Microbiol Biotechnol. 21(5): 529-35 (2011). |
| | | Aptamers | Davis K A et al. Staining of cell surface human CD4 with 2′-F-pyrimidine-containing RNA |
| | | to CD4 | aptamers for flow cytometry. Nucleic Acids Research. 26(17): 3915-3924 (1998). |
| | | | Zhou Q et al. Aptamer-containing surfaces for selective capture of CD4 expressing cells. |
| | | | Langmuir. 28(34): 12544-9 (2012). |
| | | | Zhao N et al. Blocking interaction of viral gp120 and CD4-expressing T cells by single- |
| | | | stranded DNA aptamers. Int J Biochem Cell Biol. 51: 10-8 (2014). |
| | | | Peng Z et al. Combination of an Aptamer Probe to CD4 and Antibodies for Multicolored |
| | | | Cell Phenotyping. American Journal of Clinical Pathology, 134(4): 586-593 (2010). |
| | | | Cong-Qiu C et al. CD4 Aptamer-RORγt shRNA Chimera Inhibits IL-17 Synthesis By |
| | | | Human CD4⁺ T cells. American College of Rheumatology 2014 Annual Meeting Abstract |
| | | | Number 1751 (2014). |
| | CXCR3 | see above | see above |
| Natural | CD56 | Antibody | Whiteman et al. Lorvotuzumab mertansine, a CD56-targeting antibody-drug conjugate |
| Killer Cells | | to CD56 | with potent antitumor activity against small cell lung cancer in human xenograft models. |
| | | | MAbs. 6(2): 556-66 (2014). |
| | | | Shah et al. Phase I study of IMGN901, a CD56-targeting antibody-drug conjugate, in |
| | | | patients with CD56-positive solid tumors. Invest New Drugs. 34: 290-299 (2016). |
| | | | Feng et al. Differential killing of CD56-expressing cells by drug-conjugated human |
| | | | antibodies targeting membrane-distal and membrane-proximal non-overlapping epitopes. |
| | | | MAbs. 8(4): 799-810 (2016). |
| | | | Galli et al. In Vivo Imaging of Natural Killer Cell Trafficking in Tumors. J Nucl Med. |
| | | | 56(10): 1575-80 (2015). |
| | | | Merkt et al. Peripheral blood natural killer cell percentages in granulomatosis with |
| | | | polyangiitis correlate with disease inactivity and stage. Arthritis Res Ther. 17: 337 (2015). |
| | | | Park et al. Gene expression analysis of ex vivo expanded and freshly isolated NK cells from |
| | | | cancer patients. J Immunother. 33(9): 945-55 (2010). |
| | | | Kimura et al. Tumor-draining lymph nodes of primary lung cancer patients: a potent source |
| | | | of tumor-specific killer cells and dendritic cells. Anticancer Res. 25(1A): 85-94 (2005). |
| | | | Mavoungou et al. Natural killer (NK) cell-mediated cytolysis of Plasmodium falciparum- |
| | | | infected human red blood cells in vitro. Eur Cytokine Netw. 14(3): 134-42 (2003). |
| | | | Yanagihara et al. Natural killer (NK) T cells are significantly decreased in the peripheral |
| | | | blood of patients with rheumatoid arthritis (RA). Clin Exp Immunol. 118(1): 131-6 (1999). |
| | | | Roguska et al. Humanization of murine monoclonal antibodies through variable domain |
| | | | resurfacing. Proc Natl Acad Sci USA. 91(3): 969-73 (1994). |
| | | | Nitta et al. Involvement of CD56 (NKH-1/Leu-19 antigen) as an adhesion molecule in |
| | | | natural killer-target cell interaction. J Exp Med. 170(5): 1757-61 (1989). |
| | CD2 | Antibody | Listed in Table 3C |
| | | to CD2 | |
| Macrophages | CD14 | Antibody | Spek et al. Treatment with an anti-CD14 monoclonal antibody delays and inhibits |
| | | to CD14 | lipopolysaccharide-induced gene expression in humans in vivo. J Clin Immunol. 23(2): 132- |
| | | | 40 (2003). |
| | | | Nakamura et al. Anti-human CD14 monoclonal antibody improves survival following |
| | | | sepsis induced by endotoxin, but not following polymicrobial infection. Eur J Pharmacol. |
| | | | 806: 18-24 (2017). |
| | | | Egge et al. The anti-inflammatory effect of combined complement and CD14 inhibition is |
| | | | preserved during escalating bacterial load. Clin Exp Immunol. 181(3): 457-67 (2015). |
| | | | Yidrim et al. Galectin-2 induces a proinflammatory, anti-arteriogenic phenotype in |
| | | | monocytes and macrophages. PLoS One. 10(4): e0124347 (2015). |
| | | | Hermansson et al. Macrophage CD14 expression in human carotid plaques is associated |
| | | | with complicated lesions, correlates with thrombosis, and is reduced by angiotensin |
| | | | receptor blocker treatment. Int Immunopharmacol. 22(2): 318-23 (2014). |
| | | | Genth-Zotz et al. The anti-CD14 antibody IC14 suppresses ex vivo endotoxin stimulated |
| | | | tumor necrosis factor-alpha in patients with chronic heart failure. Eur J Heart Fail. |
| | | | 8(4): 366-72 (2006). |
| | | | Olszyna et al. Effect of IC14, an anti-CD14 antibody, on plasma and cell-associated |
| | | | chemokines during human endotoxemia. Eur Cytokine Netw. 14(3): 158-62 (2003). |
| | | | Bondeson et al. The role of synovial macrophages and macrophage-produced cytokines in |
| | | | driving aggrecanases, matrix metalloproteinases, and other destructive and inflammatory |
| | | | responses in osteoarthritis. Arthritis Res Ther. 8(6): R187 (2006). |
| | | | Streit et al. 3D parallel coordinate systems--a new data visualization method in the context |
| | | | of microscopy-based multicolor tissue cytometry. Cytometry A. 69(7): 601-11 (2006). |
| | | | Ueki et al. Self-heat shock protein 60 induces tumour necrosis factor-alpha in monocyte- |
| | | | derived macrophage: possible role in chronic inflammatory periodontal disease. Clin Exp |
| | | | Immunol. 127(1): 72-7 (2002). |
| | CD11b | Antibodies | Gordon et al. Both anti-CD11a(LFA-1) and anti-CD11b (MAC-1) therapy delay the onset |
| | | to CD11b | and diminish the severity of experimental autoimmune encephalomyelitis. J Neroimmunol. |
| | | | 62(2): 153-160 (1995). |
| | | | Nakagawa et al. Optimum immunohistochemical procedures for analysis of macrophages in |
| | | | human and mouse formalin fixed paraffin-embedded tissue samples. J Clin Exp Hematop. |
| | | | 57(1): 31-36 (2017). |
| | | | Duarte et al. Generation of Immunity against Pathogens via Single-Domain Antibody- |
| | | | Antigen Constructs. J Immunol. 197(12): 4838-4847 (2016). |
| | | | Lau et al. Myeloperoxidase mediates neutrophil activation by association with |
| | | | CD11b/CD18 integrins. Proc Natl Acad Sci USA. 102(2): 431-6 (2005). |
| | | | May et al. Urokinase receptor surface expression regulates monocyte adhesion in acute |
| | | | myocardial infarction. Blood. 100(10): 3611-7 (2002). |
| | | | Ribbens et al. CD40-CD40 ligand (CD154) engagement is required but may not be |
| | | | sufficient for human T helper 1 cell induction of interleukin-2- or interleukin-15-driven, |
| | | | contact-dependent, interleukin-1beta production by monocytes. Immunology. 99(2): 279-86 |
| | | | (2000). |
| | | | Olivieri et al. Increased neutrophil adhesive capability in Cohen syndrome, an autosomal |
| | | | recessive disorder associated with granulocytopenia. Haematologica. 83(9): 778-82 (1998). |
| | | | Rambaldi et al. Innovative two-step negative selection of granulocyte colony-stimulating |
| | | | factor-mobilized circulating progenitor cells: adequacy for autologous and allogeneic |
| | | | transplantation. Blood. 91(6): 2189-96 (1998). |
| | | | Lechner et al. Peripheral blood mononuclear cells from neovascular age-related macular |
| | | | degeneration patients produce higher levels of chemokines CCL2 (MCP-1) and CXCL8 |
| | | | (IL-8). J Neuroinflammation. 14(1): 42 (2017). |
| | | | Mizee et al. Isolation of primary microglia from the human post-mortem brain: effects of |
| | | | ante- and post-mortem variables. Acta Neuropathol Commun. 17; 5(1): 16 (2007). |
| | CD40 | Antibodies | French et al. CD40 antibody evokes a cytotoxic T-cell response that eradicates lymphoma |
| | | to CD40 | and bypasses T-cell help. Nature Medicine. 5: 548-553 (1999). |
| | | | Beatty et al. CD40 Agonists Alter Tumor Stroma and Show Efficacy Against Pancreatic |
| | | | Carcinoma in Mice and Humans. Science. 331(6024): 1612-1616 (2011). |
| | | | Velasquez et al. Targeting Mycobacterium tuberculosis Antigens to Dendritic Cells via the |
| | | | DC-Specific-ICAM3-Grabbing-Nonintegrin Receptor Induces Strong T-Helper 1 Immune |
| | | | Responses. Front Immunol. 9: 471 (2018). |
| | | | McDonnell et al. Serial immunomonitoring of cancer patients receiving combined |
| | | | antagonistic anti-CD40 and chemotherapy reveals consistent and cyclical modulation of T |
| | | | cell and dendritic cell parameters. BMC Cancer. 17(1): 417 (2017). |
| | | | Dahan et al. Therapeutic Activity of Agonistic, Human Anti-CD40 Monoclonal Antibodies |
| | | | Requires Selective FcγR Engagement. Cancer Cell. 29(6): 820-831 (2016). |
| | | | Bankert et al. Induction of an altered CD40 signaling complex by an antagonistic human |
| | | | monoclonal antibody to CD40. J Immunol. 194(9): 4319-27 (2015). |
| | | | Pinelli et al. Novel insights into anti-CD40/CD154 immunotherapy in transplant tolerance. |
| | | | Immunotherapy. 7(4): 399-410 (2015). |
| | | | Bajor et al. Immune activation and a 9-year ongoing complete remission following CD40 |
| | | | antibody therapy and metastasectomy in a patient with metastatic melanoma. Cancer |
| | | | Immunol Res. 2(11): 1051-8 (2014). |
| | | | Beatty et al. A phase I study of an agonist CD40 monoclonal antibody (CP-870, 893) in |
| | | | combination with gemcitabine in patients with advanced pancreatic ductal adenocarcinoma. |
| | | | Clin Cancer Res. 19(22): 6286-95 (2013). |
| | | | Ruter et al. Immune modulation with weekly dosing of an agonist CD40 antibody in a |
| | | | phase I study of patients with advanced solid tumors. Cancer Biol Ther. 10(10): 983-93 |
| | | | (2010). |
| NKT-cells | T cell | Antibody | Tachibana et al. Increased IntratumorVA24-Positive Natural Killer T Cells: A Prognostic |
| | receptor | to T cell | Factor for Primary Colorectal Carcinomas. Clin Can Res. 11(20), 7322-27 (2005). |
| | Vα24 | receptor | Nair et al. Type II NKT-TFH cells against Gaucher lipids regulate B-cell immunity and |
| | | Vα24 | inflammation. Blood. 125(8): 1256-1271 (2015). |
| | | | Nieda et al. Therapeutic activation of V24V11 NKT cells in human subjects results in |
| | | | highly coordinated secondary activation of acquired and innate immunity. Blood. 103: 383- |
| | | | 389 (2004). |
| | CD56 | Antibody | Listed in Table 3C |
| | | to CD56 | |
| Neutrophil | CD15 | Antibody | Ball et al. Initial trial of bispecific antibody-mediated immunotherapy of CD15-bearing |
| | | to CD15 | tumors: cytotoxicity of human tumor cells using a bispecific antibody comprised of anti- |
| | | | CD15 (MoAb PM81) and anti-CD64/Fc gamma RI (MoAb 32). J Haematother. 1(1); 85-94 |
| | | | (1992). |
| | | | Rubin et al. A combination of anti-CD15 monoclonal antibody PM-81 and 4- |
| | | | hydroperoxycyclophosphamide augments tumor cytotoxicity while sparing normal |
| | | | progenitor cells. J Haematother. 3(2), 121-27 (1994). |
| Basophils | 2D7 | Antibody | Siracusa et al. Basophils and allergic inflammation. J Allergy Clin Immunol. 132(4); 789-98 |
| | | to 2D7 | (2013). |
| | | | Agis et al. Enumeration and immunohistochemical characterisation of bone marrow |
| | | | basophils in myeloproliferative disorders using the basophil specific monoclonal antibody |
| | | | 2D7. J Clin Pathol 59: 396-402 (2006). |
| | | | Raap et al. Human basophils are a source of and are differentially activated by IL-31. Clin |
| | | | Exp Allergy. Vol 47(4): 499-508 (2017). |
| | CD203c | Antibody | MacGlashan Jr. Expression of CD203c and CD63 in Human Basophils: Relationship to |
| | | to CD203c | Differential Regulation of Piecemeal and Anaphylactic Degranulation Processes. Clin Exp |
| | | | Allergy. 40(9): 1365-1377 (2010). |
| | | | Gernez et al. Basophil CD203c Levels Are Increased at Baseline and Can Be Used to |
| | | | Monitor Omalizumab Treatment in Subjects with Nut Allergy. Int Arch Allergy Immunol |
| | | | 154: 318-327 (2011). |
| | | | Khanolkar et al. Evaluation of CCR3 as a Basophil Activation Marker. Am J Clin Pathol |
| | | | 140: 293-300 (2013). |
| | FcεRIα | Antibody | Listed in Table 10 |
| | | to FcεRIα | |
| Eosinophils | CD193 | Antibody | Takeda Y et al. Augmentation of the expression of the eotaxin receptor on duodenal |
| | | to CD193 | neutrophils by IL-21. Cytokine 110: 194-203 (2018). |
| | Siglec-8 | Antibody | Yu H et al. Siglec-8 and Siglec-9 binding specificities and endogenous airway ligand |
| | | to Siglec-8 | distributions and properties. Glycobiology. 27(7): 657-668 (2017). |
| | EMR1 | Antibody | Legrand F et al. The eosinophil surface receptor epidermal growth factor-like module |
| | | to EMR1 | containing mucin-like hormone receptor 1 (EMR1): a novel therapeutic target for |
| | | | eosinophilic disorders. J Allergy Clin Immunol. 133(5): 1439-47 (2014). |
| γδ T-cells | γδ TCR | Antibodies | Vantourout P and Hayday A. Six-of-the-best: unique contributions of γδ T cells to |
| | | to γδ TCR | immunology. Nat Rev Immunol. 13(2): 88-100 (2013). |
| | | | Hayday A and Tigelaar R. Immunoregulation in the tissues by gammadelta T cells. Nat Rev |
| | | | Immunol. 3(3): 233-42 (2003). |
| | | | Hayday AC. γδ cells: a right time and a right place for a conserved third way of protection. |
| | | | Annu Rev Immunol. 18: 975-1026 (2000). |
| Engineered | Marker | Antibody | Examples of marker antigens, including LNGFR or CD20. |
| immune cells | antigen, | to marker | The marker antigen may also be an antigen expressed by the engineered immune cell (for |
| (e.g., CAR-T | eg. | antigen | example a T cell antigen, if a CAR T-cell is used). |
| cells) | CD20, | | |
| | LNGFR, | | |
| | or scFv | | |
| | fragment | | |

C. Targeting Moiety Capable of Targeting the Cancer

The targeting moiety functions in the first component comprising a targeted immune cell engaging agent by delivering the agent to the local environment of the cancer cells, enabling a localized treatment strategy. In certain embodiments, the targeting moiety targets the cancer cells by specifically binding to the cancer cells. In some instances, the targeting moiety specifically binds the cancer cells even while the inert binding partner is binding the first immune cell engaging domain.

In certain embodiments, the targeting moiety is an antibody or antigen-binding fragment thereof. By antigen-binding fragment, we mean any antibody fragment that retains its binding activity to the target on the cancer cell, such as an scFv or other functional fragment including an immunoglobulin devoid of light chains, VHH, VNAR, Fab, Fab′, F(ab′)₂, Fv, antibody fragment, diabody, scAB, single-domain heavy chain antibody, single-domain light chain antibody, Fd, CDR regions, or any portion or peptide sequence of the antibody that is capable of binding antigen or epitope. VHH and VNAR are alternatives to classical antibodies and even though they are produced in different species (camelids and sharks, respectively), we will also include them in antigen-binding fragments of antibodies. Unless specifically noted as “full length antibody,” when the application refers to antibody it inherently includes a reference to an antigen-binding fragment thereof.

Certain antibody targets (with examples of cancer cell types in parentheses) may include: Her2/Neu (Epithelial malignancies); CD22 (B cells, autoimmune or malignant); EpCAM (CD326) (Epithelial malignancies); EGFR (epithelial malignancies); PSMA (Prostate Carcinoma); CD30 (B cell malignancies); CD20 (B cells, autoimmune, allergic or malignant); CD33 (Myeloid malignancies); membrane lgE (Allergic B cells); lgE Receptor (CD23) (Mast cells or B cells in allergic disease), CD80 (B cells, autoimmune, allergic or malignant); CD86 (B cells, autoimmune, allergic or malignant); CD2 (T cell or NK cell lymphomas); CA125 (multiple cancers including Ovarian carcinoma); Carbonic Anhydrase IX (multiple cancers including Renal Cell Carcinoma); CD70 (B cells, autoimmune, allergic or malignant); CD74 (B cells, autoimmune, allergic or malignant); CD56 (T cell or NK cell lymphomas); CD40 (B cells, autoimmune, allergic or malignant); CD19 (B cells, autoimmune, allergic or malignant); c-met/HGFR (Gastrointestinal tract and hepatic malignancies; TRAIL-R1 (multiple malignancies including ovarian and colorectal carcinoma); DRS (multiple malignancies including ovarian and colorectal carcinoma); PD-1 (B cells, autoimmune, allergic or malignant); PD1L (Multiple malignancies including epithelial adenocarcinoma); IGF-1R (Most malignancies including epithelial adenocarcinoma); VEGF-R2 (The vasculature associated with the majority of malignancies including epithelial adenocarcinomas; Prostate stem cell antigen (PSCA) (Prostate Adenocarcinoma); MUC1 (Epithelial malignancies); CanAg (tumors such as carcinomas of the colon and pancreas); Mesothelin (many tumors including mesothelioma and ovarian and pancreatic adenocarcinoma); P-cadherin (Epithelial malignancies, including breast adenocarcinoma); Myostatin (GDF8) (many tumors including sarcoma and ovarian and pancreatic adenocarcinoma); Cripto (TDGF1) (Epithelial malignancies including colon, breast, lung, ovarian, and pancreatic cancers); ACVRL 1/ALK1 (multiple malignancies including leukemias and lymphomas); MUC5AC (Epithelial malignancies, including breast adenocarcinoma); CEACAM (Epithelial malignancies, including breast adenocarcinoma); CD137 (B cells or T cells, autoimmune, allergic or malignant); CXCR4 (B cells or T cells, autoimmune, allergic or malignant); Neuropilin 1 (Epithelial malignancies, including lung cancer); Glypicans (multiple cancers including liver, brain and breast cancers); HER3/EGFR (Epithelial malignancies); PDGFRa (Epithelial malignancies); EphA2 (multiple cancers including neuroblastoma, melanoma, breast cancer, and small cell lung carcinoma); CD38 (Myeloma); CD138 (Myeloma); α4-integrin (AML, myeloma, CLL, and most lymphomas).

In certain modes, antibodies include an anti-epidermal growth factor receptor antibody such as Cetuximab, an anti-Her2 antibody, an anti-CD20 antibody such as Rituximab, an anti-CD22 antibody such as Inotuzumab, G544 or BU59, an anti-CD70 antibody, an anti-CD33 antibody such as hp67.6 or Gemtuzumab, an anti-MUC1 antibody such as GP1.4 and SM3, an anti-CD40 antibody, an anti-CD74 antibody, an anti-P-cadherin antibody, an anti-EpCAM antibody, an anti-CD138 antibody, an anti-E-cadherin antibody, an (anti-CEA antibody, an anti-FGFR3 antibody, and an anti a4-integrin antibody such as natalizumab.

Table 3A provides nonlimiting examples of cancer types, possible targeting moieties, and proteases that are expressed by those cancer types. A protease associated with a cancer may be termed a tumor-associated protease. In order to prepare an ATTAC, the cancer may be identified, and a target chosen for the targeting moiety (as desired), and one or two proteases chosen for the cancer type, as well (as desired).

TABLE 3A
Coordination of Cancer Type, Targets for Targeting Moiety, and
Proteases that Can Cleave Cleavage Sites

| | | Proteases that can |
|---|---|---|
| | | Cleave Cleavage |
| Cancer | Targets for Targeting Moiety | Site |
| Prostate | ADAM17, CD59, EpCAM, HER2, | KLK2, KLK3 (PSA), |
| Cancer | Integrin αV, Integrin αVβ3, MCP-1, | KLK4, ADAM17, |
| | PCLA, PSCA, PSMA, RANKL, RG1, | Cathepsin B, uPA, |
| | SLC44A4 STEAP-1, VEGF-C | uPAR, HPN, ST14, |
| | | TMPRSS2 |
| Breast | CA125, CCN1, CD44, CD98, c-RET, | MMP2, MMP9, |
| Cancer | DLL4, EpCAM, Episialin, GPNMB, | Cathepsin L, |
| | HER2/neu, HER3, IGF-1R, Integrin | Cathepsin K, |
| | α6β4, LFL2, LIV-1, Ly6E, MUC1, | Cathepsin B, |
| | MUC18, NRP1, Phosphatidylserine, | MMP11, HPN, |
| | PRLR, TACSTD-2, Tenascin C, | ST14, ADAM28 |
| | TWEAKR, VANGL2, PD-L1, PD-L2 | |
| Myeloma | BCMA, IGF-1R, DKK-1, ICAM-1, | MMP2, MMP9, |
| | CD138/Syndecan1, CD38, GRP78, | MMP1, MMP7, |
| | FGFR3, SLAMF6, CD48, TfR(CD71) | TMPRSS2, PRSS22, |
| | APRIL, CD40, CD19, DR5, CXCR4 | KLK11 |
| B-cell | CD20, CD22, CD19, CD37, CD70, | ADAM28, Cathepsin |
| Lymphoma | HLA-DR, CD70b | B, MMP9 |
| Renal Cell | PD-L, PD-L2, CAIX, TPBG, CD70, | ST14, MMP9 |
| carcinoma | ENPP3, FGFR1 | |
| Gastric | VEGFR-2, CLDN18, GCC, C242, | MMP2, MMP9, |
| Carcinoma | HER2/neu, FGFR2, EpCAM, GPR49, | Cathepsin B, uPA, |
| | HER3, IGFR | uPAR |
| Glio- | HER2/neu, EGFR, ALK, EphA2, | MMP2, MMP9, |
| blastoma | GD2, EGFRvIII, ALK | |
| T-cell | CD2, CD4, CD5, CD71, CD30 | Cathepsin B, |
| lymphoma | | Cathepsin D, MMP9 |
| Hodgkin | CD30, CD40, IL-3Ra, CD30 | Cathepsin B |
| Lymphoma | | |
| Lung | EGFR, IGF-1R, HER3, Integrin α5β1, | Cathepsin B, MMP2, |
| Cancer | Lewis y/b antigen, EGFL7, TPBG, | MMP9, ST14, |
| | DKK-1, NaPi2b, flt4, cMet, CD71 | ADAM17 |
| Pancreatic | SLC44A4, uPAR, MUC1, MUCH16, | Cathepsin B, ST14, |
| Carcinoma | TACSTD-2, CEA, EphhA4, | ADAM28 |
| | mesothelin, EGFR, MUC13, MU5AC, | |
| | AGF-1R, HER3, CD71 | |
| Head and | EGFR, EpCAM, HER2 | Cathepsin B, ST14, |
| Neck | | ADAM17 |
| cancer | | |
| Acute | CD33, CD133, CD123, CD45, CD98, | ADAM17, Cathepsin |
| myeloid | c-Kit, Lewis Y, Siglec-15, FLT-3 | B, uPA, uPAR |
| leukemia | | |
| Melanoma | MUC18, CD40, GD2, CEACAM1, | Cathepsin B, MMP9 |
| | Cadherin-19, GM3, Integrin α5β1, | |
| | TYRP1, GD3, Integrin αV | |
| Ovarian | HER2/neu, EpCAM, CA125, DLL4, | Cathepsin B, MMP2, |
| Cancer | Integrin αVβ3, MUC5A, NaPi2B, | MMP9 |
| | Mesothelin, CLDN6 | |
| Liver | Glypican-3, FGFR4, ENPP3, | Cathepsin B, MMP9 |
| Cancer | PIVKA-II, PLVAP, cMet, EpCAM | |
| Colorectal | EGFR, Lewis y/b, Progastrin, GPR49, | Cathepsin S, |
| Carcinoma | CEA, CLDN1, A33, CK8, Integrin | Cathepsin L, |
| | αV, EpCAM, DLL4, EGFL7, FAP, | Cathepsin B, uPA, |
| | | uPAR, MMP2, |
| | | MMP9, ST14 |

Table 3B provide additional information about cancers that may be targeting with different targeting moieties, including the fact that some targeting moieties may be able to target a number of different types of cancer. In an ATTAC, the first component would comprise a targeting moiety capable of targeting a cancer.

TABLE 3B
Potential Targeting Moieties

| Targeting Moiety for | |
|---|---|
| First Component | Cancer Type |
| Antibody against CD20 | Lymphoma |
| (such as Rituximab) | |
| Antibody against CD80 | Lymphoma |
| Antibody against CD22 | Lymphoma |
| (such as Inotuzumab) | |
| Antibody against CD70 | Lymphoma |
| Antibody against CD30 | Lymphoma (Hodgkin, T-cell, and B-cell) |
| Antibody against CD19 | Lymphoma |
| Antibody against CD74 | Lymphoma |
| Antibody against CD40 | Lymphoma |
| Antibody against HER2 | Epithelial malignancies, breast cancer, |
| | sarcoma |
| Antibody against EpCAM | Epithelial malignancies, hepatocellular |
| | carcinoma, lung cancer, pancreatic cancer, |
| | colorectal carcinoma |
| Antibody against EGFR | Breast cancer, epithelial malignancies, |
| (such as Cetuximab) | gliomas, lung cancer, colorectal carcinoma, |
| | ovarian carcinoma, brain cancer |
| Antibody against mucin | Breast cancer |
| protein core | |
| Antibody against | Gliomas |
| transferrin receptor | |
| Antibody against | Drug-resistant melanomas |
| gp95/gp97 | |
| Antibody against p- | Drug-resistant melanomas |
| glycoprotein | |
| Antibody against TRAIL- | Multiple malignancies, including |
| R1 | ovarian and colorectal carcinoma |
| Antibody against DR5 | Multiple malignancies, including |
| | ovarian and colorectal carcinoma |
| Antibody against IL-4 | Lymphomas and leukemias |
| Antibody against IL-6 | Lymphomas and leukemias |
| Antibody against PSMA | Prostate carcinoma |
| Antibody against PSCA | Prostate carcinoma |
| Antibody against P- | Epithelial malignancies |
| cadherin (CDH3) | |
| Antibody against LI- | Gastrointestinal malignancies |
| cadherin (CDH17) | |
| Antibody against | Epithelial malignancies |
| CEACAM5 | |
| Antibody against | Epithelial malignancies |
| CEACAM6 | |
| Antibody against | Epithelial malignancies |
| CEACAM7 | |
| Antibody against | Epithelial malignancies |
| TMPRSS4 | |
| Antibody against CA9 | Epithelial malignancies |
| Antibody against GPA33 | Epithelial malignancies |
| Antibody against STEAP1 | Epithelial malignancies, particularly prostate |
| Antibody against CLDN6 | Epithelial malignancies, particularly ovarian |
| Antibody against CLDN16 | Epithelial malignancies, particularly ovarian |
| Antibody against LRRC15 | Epithelial malignancies |
| Antibody against TREM2 | Epithelial malignancies |
| Antibody against CLDN18 | Epithelial malignancies, particularly |
| | pancreatic |
| Antibody against Cripto | Epithelial malignancies |
| (TDGF1) | |
| Antibody against PD1L | Epithelial adenocarcinoma |
| Antibody against IGF-1R | Epithelial adenocarcinoma |
| Antibody against CD38 | Myeloma |
| Antibody against BCMA | Myeloma |
| Antibody against CD138 | Myeloma |
| Antibody against CD33 | Myeloid malignancies, such as AML |
| Antibody against CD37 | B-cell malignancies |
| Antibody against CD123 | Myeloid malignancies such as AML |
| Antibody against CD133 | Myeloid malignancies such as AML |
| Antibody against CD49d | Myeloid malignancies such as AML |
| Antibody against Glypican | Hepatocellular carcinoma |
| 3 | |
| Antibody against TM4SF5 | Hepatocellular carcinoma, pancreatic cancer |
| Antibody against cMet | Hepatocellular carcinoma |
| Antibody against MUC1 | Pancreatic cancer, ovarian carcinoma |
| Antibodies against | Pancreatic, ovarian and epithelial cancers |
| mesothelin (MSLN) | and mesothelioma |
| Antibody against GD2 | Sarcoma, brain cancers |
| Antibody against HER3 | Breast cancer |
| Antibody against IL-13R | Brain cancer |
| Antibody against DLL3 | Small-cell carcinoma, brain cancer |
| Antibody against MUC16 | Ovarian cancer |
| Antibodies against TFR2 | Liver cancer |
| Antibodies against TCR | T-cell malignancies |
| B1 or TCRB2 constant | |
| region | |
| Antibodies against TSHR | Thyroid malignancies |

Antibodies that have bind tumor antigens and that have specificity for tumor cells are well-known in the art. Table 3C summarizes selected publications on exemplary antibodies that bind tumor antigens and that could be used as targeting moieties in the invention.

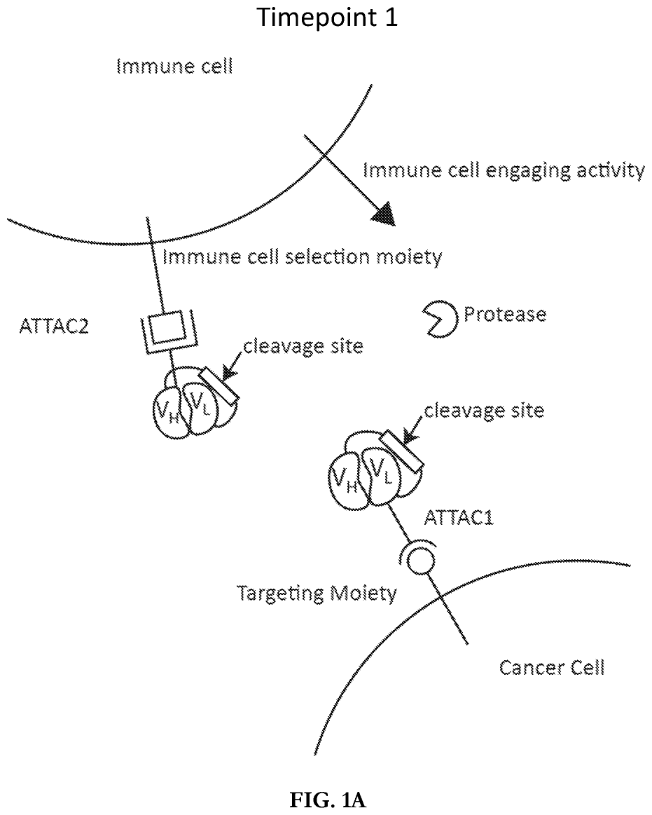

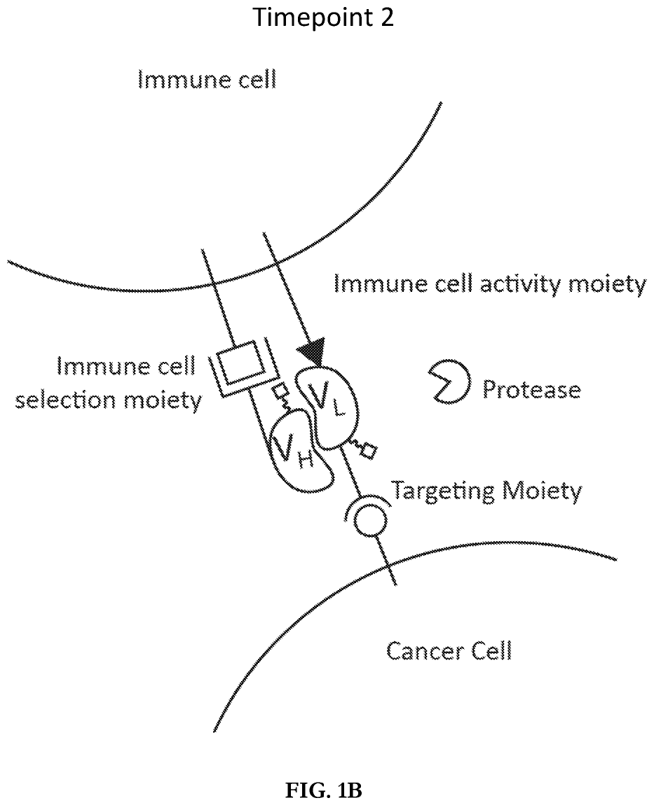

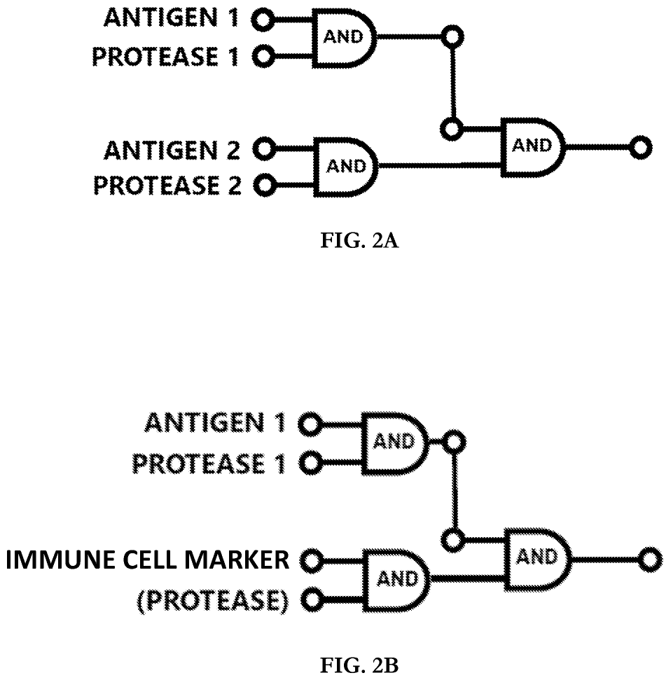

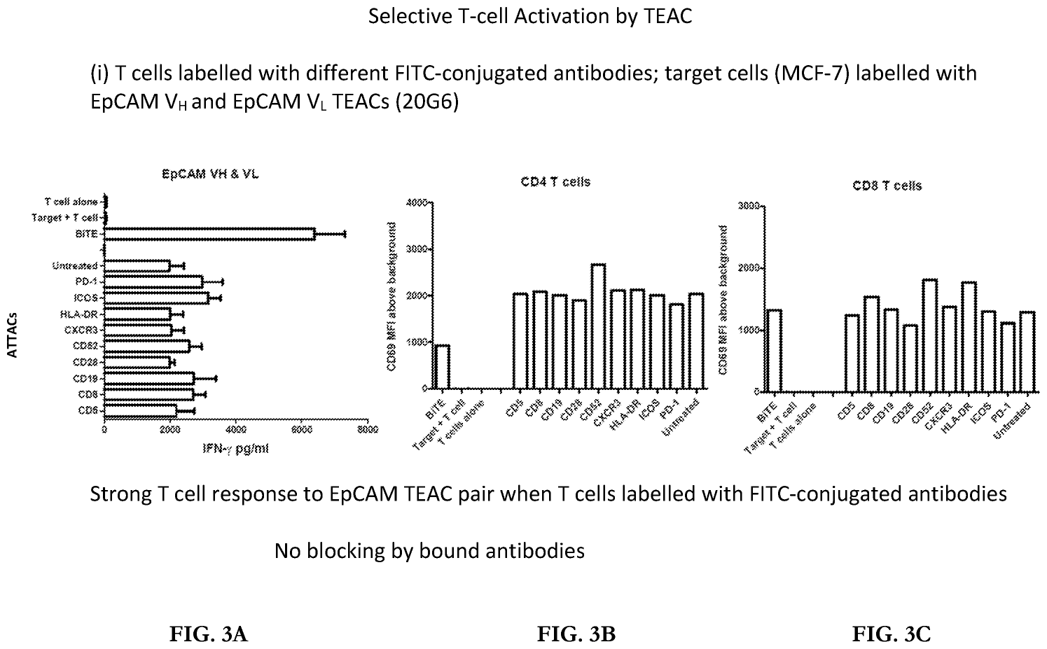

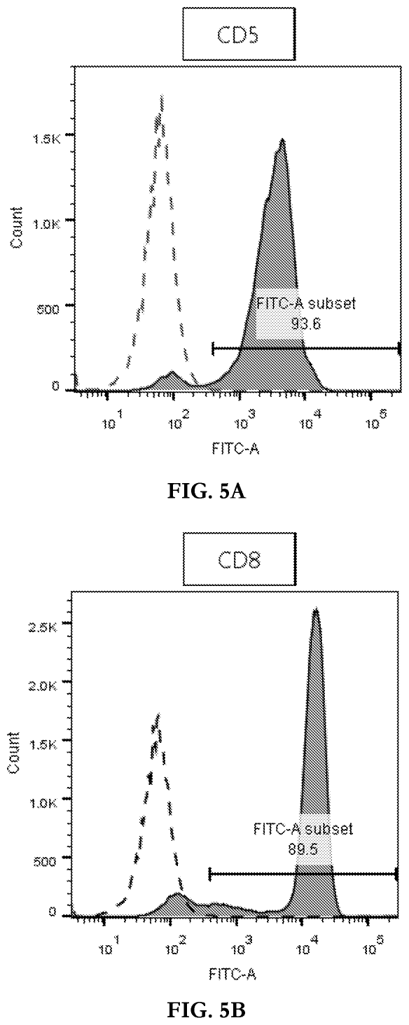

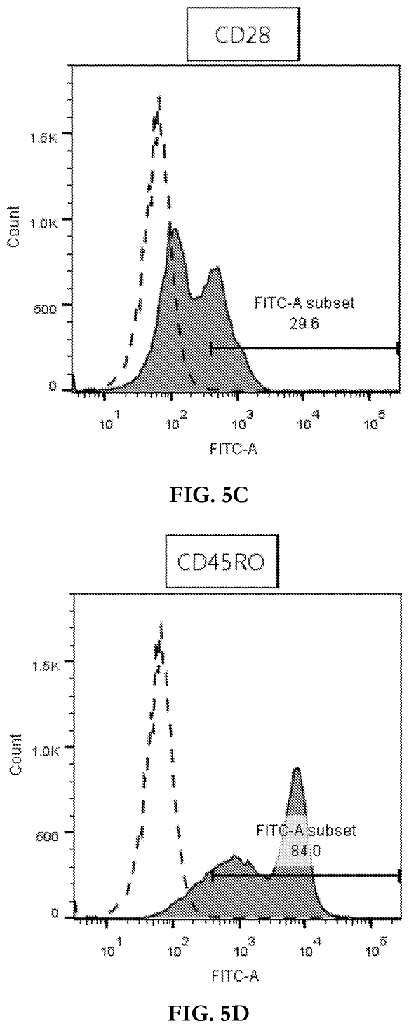

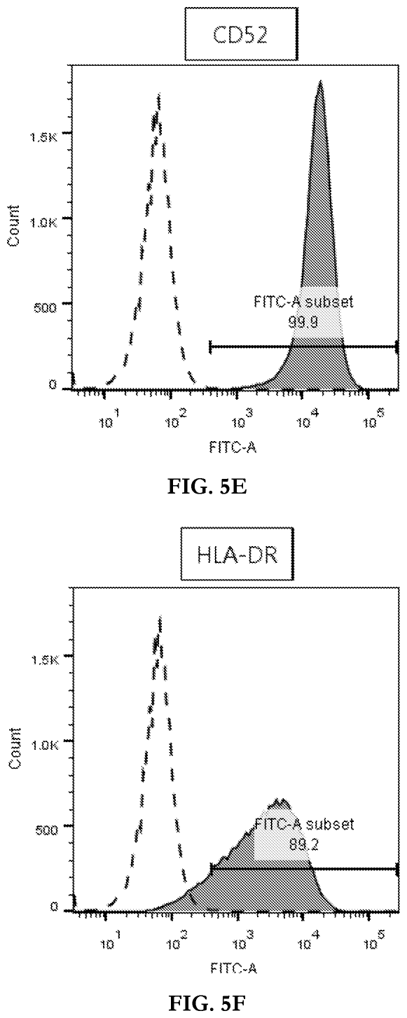

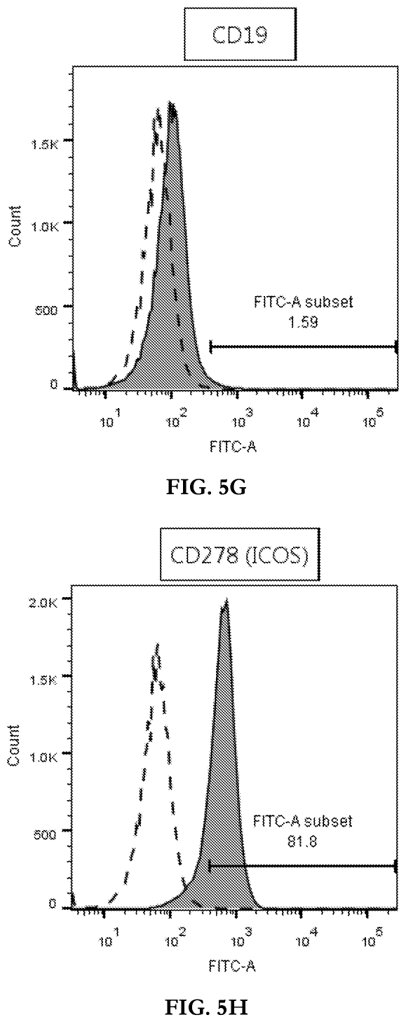

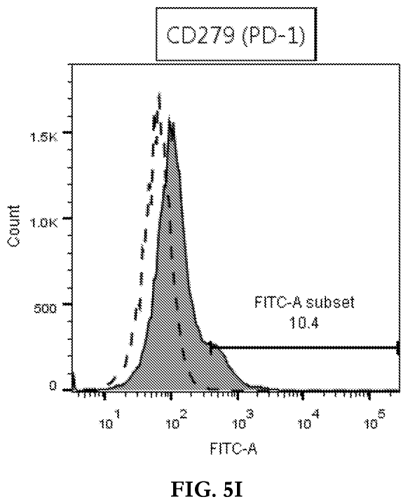

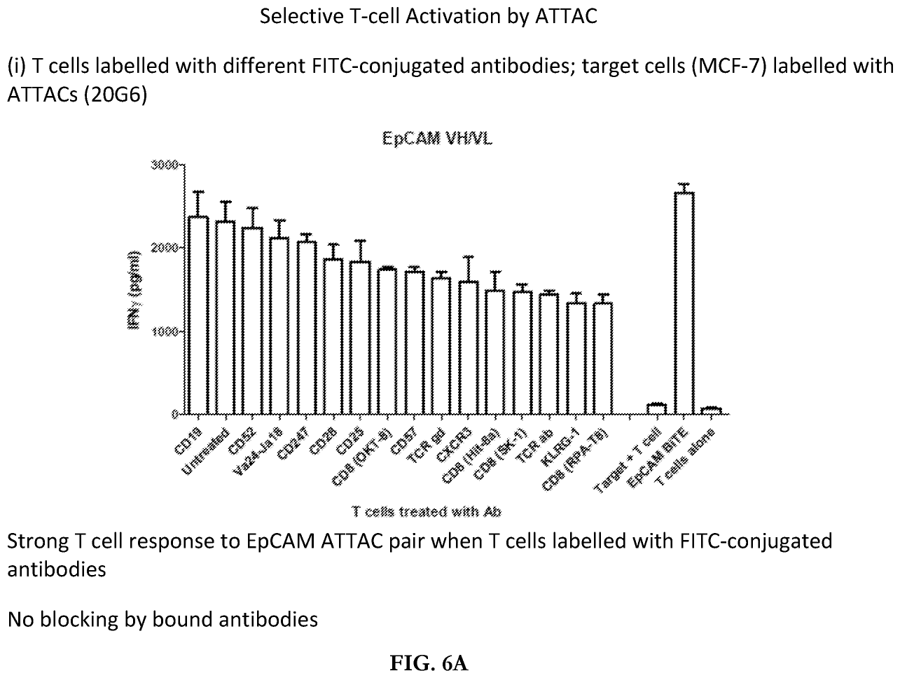

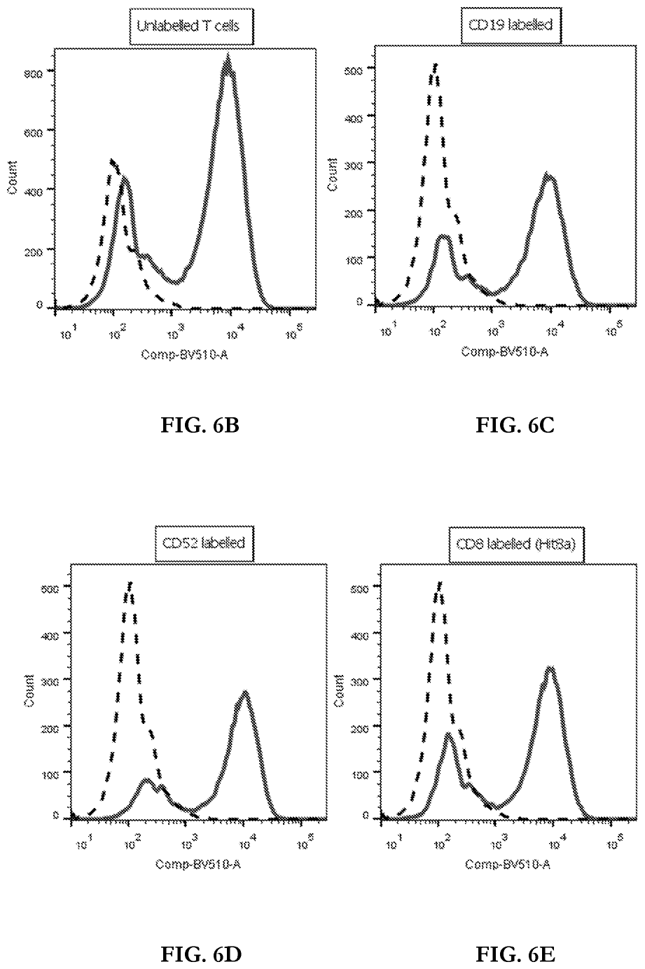

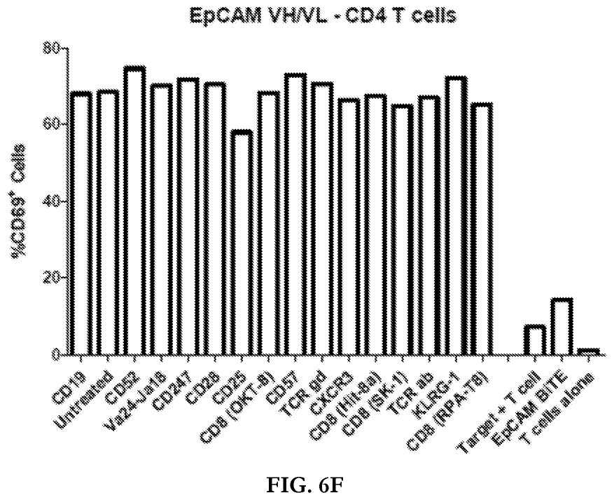

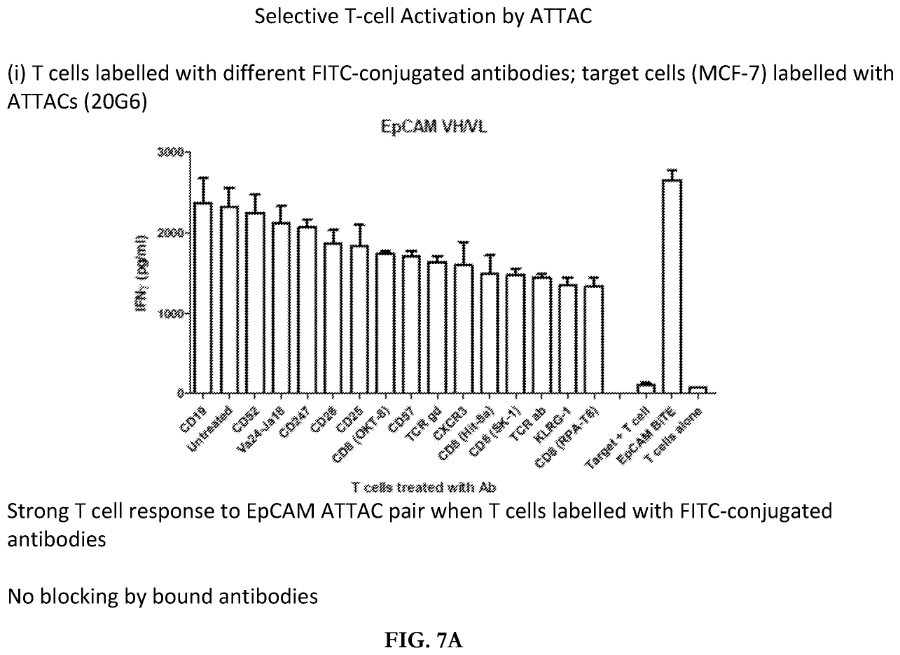

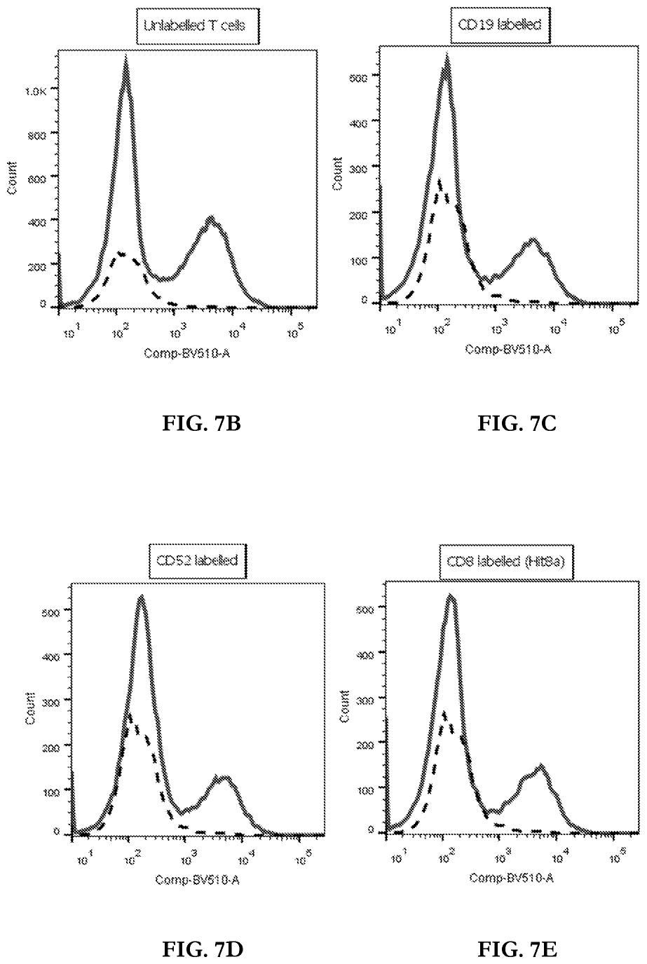

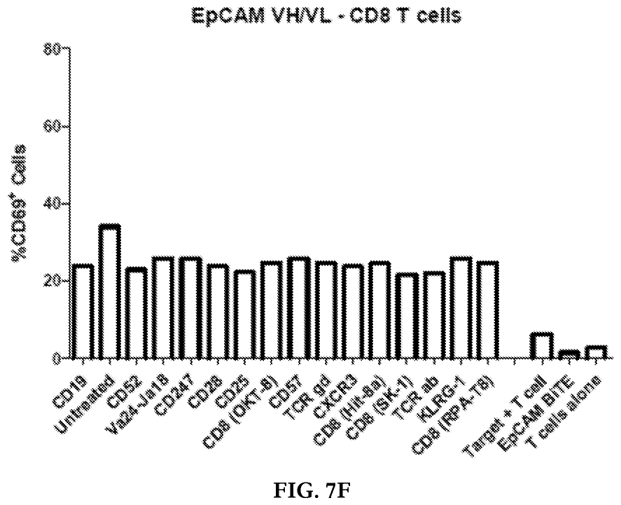

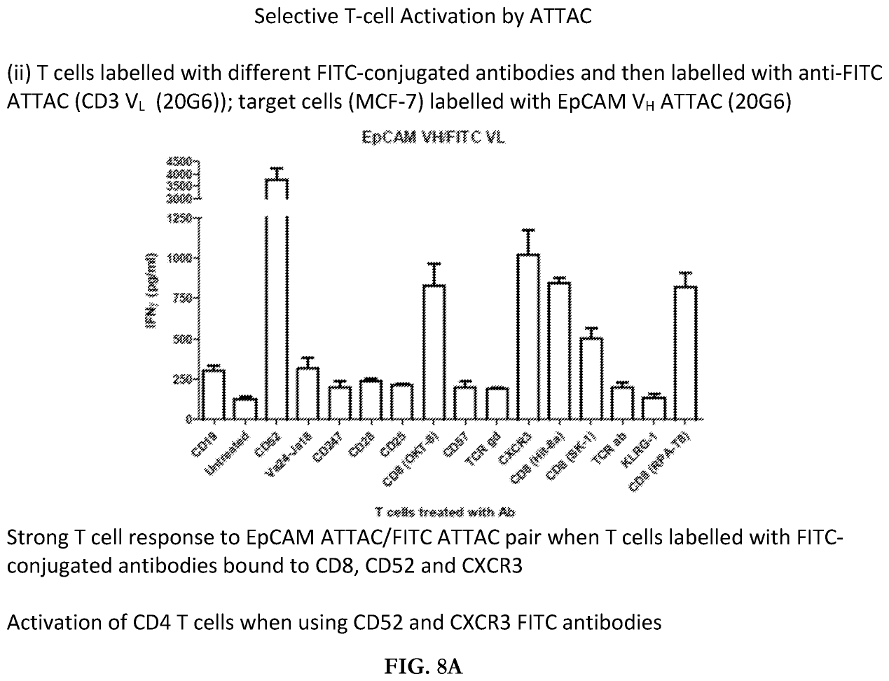

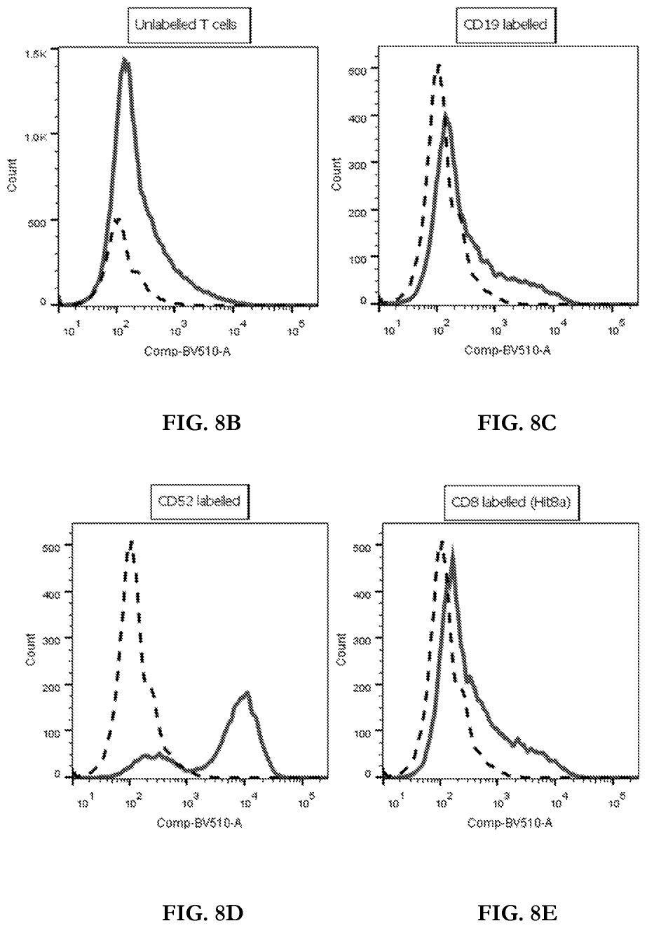

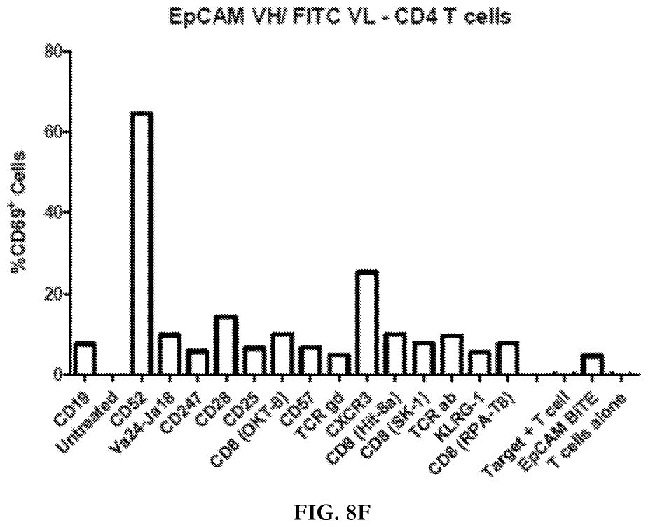

TABLE 3C
Selected publications on antibodies that bind tumor antigens

| Antigen | Publications |
|---|---|
| Her2/Neu | Carter P et al., Humanization of an anti-p185HER2 antibody for human cancer therapy, Proc Natl Acad Sci USA |
| | 89(10): 4285-9 (1992). This paper discloses the heavy and light chain sequences in its FIG. 1B. |
| | US20090202546 (Composition comprising antibody that binds to domain II of her2 and acidic variants thereof). |
| | This application discloses the variable light and variable heavy chain sequences in its claim 8. |
| | Olafsen T et al., Characterization of engineered anti-p185HER-2 (scFv-CH3)2 antibody fragments (minibodies) |
| | for tumor targeting, Protein Eng Des Sel (4): 315-23 (2004). This paper discloses light and heavy chain variable |
| | region sequences in its FIG. 1. |
| EpCAM/ | WO2008122551 (Anti-epcam antibody and uses thereof). This application discloses CDR sequences in claims 1-7. |
| CD326 | WO2010142990 A1 (Anti-EpCAM Antibodies). This application discloses CDR sequences in its claims 1-5 and 7. |
| | U.S. Pat. No. 6,969,517 (Recombinant tumor specific antibody and use thereof). This application discloses |
| | light and heavy chain sequences in its claims 1-4. |
| EGFR | Garrett J et al., Antibodies specifically targeting a locally misfolded region of tumor associated EGFR, Proc Natl |
| | Acad Sci USA 106(13): 5082-5087 and pages 1-7 of Supporting Information including FIGS. S1-S5 (2009). |
| | This paper discloses CDR sequences in its Supplemental Information FIG. S1. (A). |
| | U.S. Pat. No. 5,844,093 Anti-egfr single-chain fws and anti egfr antibodies). This patent discloses CDR |
| | sequences in its FIG. 1. |
| PSMA | US20110028696 A1 (Monoclonal antibodies against prostate specific membrane antigen (psma) lacking in fucosyl |
| | residues). This application discloses CDR sequences in claims 3-4. |
| | WO2003064606 (Human monoclonal antibodies to prostate specific membrane antigen (psma)). This application |
| | discloses CDR sequences in its claim 1. |
| CA125 | WO2011119979 A2 (Antibodies to muc 16 and methods of use thereof). This application discloses VH and VL |
| | sequences in its claim 6. |
| | US20080311134 A1 (Cysteine engineered anti-muc 16 antibodies and antibody drug conjugates). FIGS. 1-4 of this |
| | application show heavy and light chain sequences. |
| Carbonic | WO2007065027 A2 (Carbonic anhydrase ix (g250) antibodies and methods of use thereof). This application |
| Anhydrase | discloses CDR sequences in its claims 4-10. |
| IX | U.S. Pat. No. 7,378,091B2 (Antibodies against carbonic anhydrase IX (CA IX) tumor antigen). This application |
| | discloses CDR sequences in its FIGS. 26-29. |
| c-met/ | US20050054019 A1 (Antibodies to c-met). This application discloses heavy and light chain sequences in its claim |
| HGFR | 6 and CDR sequences in its claim 7. |
| | US 20090175860 A1 (Compositions and methods of use for antibodies of c-Met). This application discloses |
| | CDRs in its FIGS. 1-3 and heavy and light chain sequences in its claims 12-13. |
| TRAIL- | US20040214235 A1 (Anti-trail-r antibodies). This application discloses heavy and light chain sequences in its |
| R1/DR4 | claims 54-55. |
| | US20060062786 A1 (Antibodies that immunospecifically bind to TRAIL receptors). This application discloses |
| | VH and VL sequences in its claims 1-2. |
| TRAIL- | US20070031414A1 (DR5 antibodies and uses thereof). This application discloses heavy and light chain |
| R2/DRS | sequences in its claim 1. |
| | U.S. Pat. No. 7,790,165B2 (Antibody selective for a tumor necrosis factor-related apoptosis-inducing ligand |
| | receptor and uses thereof). This application discloses heavy and light chains sequences in its claims 1-5. |
| IGF-1R | US 20040086503 A1 (Antibodies to insulin-like growth factor receptor). This application discloses light and |
| | heavy chain variable region sequences and CDR sequences in its claims 11-14. |
| | US 20070196376 A1 (Binding proteins specific for insulin-like growth factors and uses thereof). This |
| | application discloses CDR sequence data in its claims 46-47. |
| | WHO Drug Information Vol. 24, No. 2, 2010 INN PL103. This document discloses the sequence of |
| | ganitumab on pages 144-145. |
| VEGF-R2 | Rinderknecht M et al., Phage-Derived Fully Human Monoclonal Antibody Fragments to Human Vascular |
| | Endothelial Growth Factor-C Block Its Interaction with VEGF Receptor-2 and 3, PLoS One 5(8): e11941 (2010). |
| | This paper discloses CDR sequences in its Table 2. |
| | WO1998045331 A2 (Anti-VEGF antibodies). This application discloses CDR sequences in its claims 6, 8, and 9. |
| Prostate | US20090181034 A1 (Antibodies and related molecules that bind to psca proteins). This application discloses |
| stem cell | VH and VL sequences in its claim 17. |
| antigen | U.S. Pat. No. 6,790,939 B2 (Anti-PSCA antibodies). This application discloses CDR sequences in its FIG. 61. |
| (PSCA) | WO2009032949 A2 (High affinity anti-prostate stem cell antigen (psca) antibodies for cancer targeting and |
| | detection). This application discloses CDR sequences in its FIG. 2. |
| MUC1 | Thie H et al., Rise and Fall of an Anti-MUC1 Specific Antibody, PLoS One Jan 14; 6(1): e15921 (2011). |
| | This paper discloses CDR sequences in its FIG. 1. |
| | Henderikx H et al., Human Single-Chain Fv Antibodies to MUC1 Core Peptide Selected from Phage Display |
| | Libraries Recognize Unique Epitopes and Predominantly Bind Adenocarcinoma, Cancer Res. 58(19): 4324-32 |
| | (1998). This paper discloses CDR sequences in its Table 2. |
| CanAg | US20080138898 A1 (Methods for improving antibody production). This application discloses CDR |
| | sequences in its FIG. 5. |
| Mesothelin | WO2009068204 A1 (Anti-mesothelin antibodies and uses therefor). This application discloses CDR |
| | sequences in its Table 7. |
| P-cadherin | WO2010001585 A1 (Anti-CDH3 antibodies labeled with radioisotope label and uses thereof). This |
| | application discloses VH and VL variable region sequences disclosed in its paragraph |
| Myostatin/ | U.S. Pat. No. 7,632,499 B2 (Anti-myostatin antibodies). This application discloses CDR sequences in its claim 1. |
| GDF8 | US 20090148436 A1 (Antibody to GDF8 and uses thereof). This application discloses CDR, VH, and VL |
| | sequences in its claims 2-8. |
| Cripto/ | US20100008906 A1 (Cripto binding molecules). This application discloses light and heavy chain sequences |
| TDGF1 | in its paragraph in its paragraph [0492]. |
| | U.S. Pat. No. 7,531,174 B2 (Cripto blocking antibodies and uses thereof). This application discloses a list |
| | of hybridomas that secrete anti-Cripto antibodies in its Tables 1 and 2. These hybridomas were |
| | available for purchase from the ATCC. |
| MUC5AC | Chung W C et al., CREB mediates prostaglandin F2alpha-induced MUC5AC overexpression, |
| | J Immunol 182(4): 2349-56 (2009) at page 3, second paragraph discloses that clone 45M1 was an anti- |
| | MUC5AC antibody available for purchase. |
| CEACAM | Pavoni E. et al., Selection, affinity maturation, and characterization of a human scFv antibody |
| | against CEA protein, BMC Cancer 6: 41 (2006). This paper discloses CDR sequences of clone |
| | E8 in its FIG. 3. Reactivity of E8 with CEACAM is shown in its FIG. 6. |
| SLC44A4 | US20090175796 A1 (Antibodies and related molecules that bind to 24p4c12 Proteins). This application |
| (formerly | discloses light and heavy chain variable domain sequences in its FIGS. 2 and 3. |
| known as | U.S. Pat. No. 8,039,597 B2 (Antibodies and related molecules that bind to 24p4c12 Proteins). This application |
| protein | discloses light and heavy chain variable domain sequences in its claim 1 and in its FIGS. 2 and 3. |
| 24P4C12 | U.S. Pat. No. 8,309,093 B2 (Antibody drug conjugates (ADC) that bind to 24P4C12 proteins). This application |
| which | discloses light and was heavy chain variable domain sequences in its claim 1 and in its FIGS. 2 and 3. |
| renamed | US20100330107 A1 (Antibody drug conjugates (ADC) that bind to 24P4C12 proteins). This application |
| SLC44A4 | discloses light and heavy chain variable domain sequences in its claims 1 and 2, and in its FIGS. 2 and 3. |
| by the | WO2010111018 A1 (Antibody drug conjugates (ADC) that bind to 24P4C12 proteins). This application |
| Hugo | discloses light and heavy chain variable domain sequences in its claims 1 and 2, and in its FIGS. 2 and 3. |
| Convention | |
| (see | |
| US8039497 | |
| at 114: 56- | |
| 62)) | |
| Neuropilin | U.S. Pat. No. 8,318,163 B2 (Anti-pan neuropilin antibody and binding fragments thereof). This application |
| 1 | discloses light and heavy chain variable domain sequences in its claim 1 and in its FIGS. 7 and 8. |
| | WO 2008/143666 (Crystal structures of neuropilin fragments and neuropilin-antibody complexes). This |
| | application discloses light and heavy chain variable domain sequences in its claim 8 and in its FIGS. 7 and 8. |
| Glypican | U.S. Pat. No. 7,867,734 B2 (Anti-glypican 3 antibody having modified sugar chain). This application discloses |
| | the heavy chain variable region in its claim 1. CDR sequences are disclosed in Table 1 of this application. |
| | U.S. Pat. No. 7,871,613 B2 (Adjuvant therapy with the use of anti-glypican 3 antibody). This application |
| | discloses the heavy chains equence in its claim 6 and the light chain sequence in its claim 7. |
| EphA2 | US20100298545 A1. (Epha2 agonistic monoclonal antibodies and methods of use thereof). This application |
| | discloses CDR sequences in its claim 50. |
| | US20100278838 A1. (Epha2 monoclonal antibodies and methods of use thereof). This application discloses |
| | VH/VL and CDR sequences in its claim 101. |
| | US20100183618 A1 (Anti-epha2 antibody). This application discloses CDR sequences in its claim 11. |
| E-cadherin | U.S. Pat. No. 5,610,281 (Antibodies for modulating heterotypic E-cadherin interactions with human T |
| | lymphocytes). This application discloses that anti- E-cadherin clone E4.6 is available for the ATCC |
| | (HB 11996) in its claim 4. |
| CEA | WO2004032962 A1 (Combination therapy with class iii anti-cea monoclonal antibodies and therapeutic agents). |
| | This application discloses CDR sequences in its claim 6 and its claim 14. |
| | U.S. Pat. No. 5,877,293 (CDR grafted anti-CEA antibodies and their production). This application |
| | discloses antibody sequences in its claims 1-5. |
| | US20080069816 A1 (Humanized anti-cea t84.66 antibody and uses thereof). This application discloses |
| | antibody sequence in its claims 22-23. |
| FGFR3 | US20080044419 A1 (Treatment of T Cell Mediated Diseases by Inhibition of Fgfr3). This application |
| | discloses scFv sequences in claim 6 and VH/VL sequences in its claims 7-10. |
| | US20090175866 A1 (Treatment of B-cell malignancies). This application discloses Vh, Vl and CDR |
| | sequences in its claims 11-12. |
| | Martinez-Torrecuadrada J et al. Targeting the extracellular domain of fibroblast growth factor receptor 3 |
| | with human single-chain Fv antibodies inhibits bladder carcinoma cell line proliferation. Clin Cancer |
| | Res 11(17): 6280-90 (2005). This publication shows VH and VL sequences of a scFv in its FIG. 2. |
| HER3 | Lee-Hoeflich S T et al. A Central Role for HER3 in HER2-Amplified Breast Cancer: Implications for |
| | Targeted Therapy. Cancer Res. 68(14): 5878-5887 (2008). |
| | Scartozzi M et al. The role of HER-3 expression in the prediction of clinical outcome for advanced colorectal |
| | cancer patients receiving irinotecan and cetuximab. Oncologist. 16(1): 53-60 (Epub Jan. 6, 2011). |
| | Sheng Q et al. An activated ErbB3/NRG1 autocrine loop supports in vivo proliferation in ovarian cancer |
| | cells. Cancer Cell .17(3): 298-310 (2010). |
| | Schoeberl B et al. An ErbB3 antibody, MM-121, is active in cancers with ligand dependent activation. |
| | Cancer Res. 70(6): 2485-2494 (2010). |
| | Khan I H et al. Microbead arrays for the analysis of ErbB receptor tyrosine kinase activation and |
| | dimerization in breast cancer cells. Assay Drug Dev Technol. 8(1): 27-36. (2010). |
| | Robinson M K et al. Targeting ErbB2 and ErbB3 with a bispecific single-chain Fv enhances targeting selectivity |
| | and induces a therapeutic effect in vitro. British Journal of Cancer 99: 1415-1425 (2008). |
| | Reschke Metal. HER3 is a determinant for poor prognosis in melanoma. Clin Cancer Res. 14(16): |
| | 5188-97 (2008). |
| PDGFRa | Martinhoe, O. et al. Expression, mutation and copy number analysis of platelet-derived growth factor |
| | receptor A (PDGFRA) and its ligand PDGFA in gliomas. Br J Cancer 101: 973-982 (2009). |
| | Loizos N et al. Targeting the platelet-derived growth factor receptor alpha with a neutralizing human |
| | monoclonal antibody inhibits the growth of tumor xenografts: implications as a potential therapeutic target. |
| | Mol Cancer Ther. 4(3): 369-79 (2005). |
| | Russell M R et al. Targeting the {alpha} receptor for platelet-derived growth factor as a primary or combination |
| | therapy in a preclinical model of prostate cancer skeletal metastasis. Clin Cancer Res. 16(20): 5002-10 (2010). |
| | Shah G D et al. Rationale for the development of IMC-3G3, a fully human immunoglobulin G subclass 1 |
| | monoclonal antibody targeting the platelet-derived growth factor receptor alpha. Cancer. 116(4 Suppl): |
| | 1018-26 (2010). |
| | Dolloff N G et al. Human bone marrow activates the Akt pathway in metastatic prostate cells through |
| | transactivation of the alpha-platelet-derived growth factor receptor. Cancer Res. 67(2): 555-62 (2007). |
| CS1 | Tai-Y T et al. Anti-CS1 humanized monoclonal antibody HuLuc63 inhibits myeloma cell adhesion and |
| | induces antibody-dependent cellular cytotoxicity in the bone marrow milieu. |
| | Blood 112(4): 1329-1337 (2008). |
| | Van Rhee F et al. Combinatorial efficacy of anti-CS1 monoclonal antibody elotuzumab (HuLuc63) and |
| | bortezomib against multiple myeloma. Mol Cancer Ther. 8(9): 2616-2624 (2009). |
| | Hsi E D et al. CS1, a potential new therapeutic antibody target for the treatment of multiple myeloma. |
| | Clin Cancer Res. 14(9): 2775-2784 (2008). |
| | Lee J K et al. CS1 (CRACC, CD319) induces proliferation and autocrine cytokine expression on human |
| | B lymphocytes. J Immunol 179: 4672-4678 (2007). |
| CD137 | Broll K et al. CD137 Expression in Tumor Vessel Walls: High Correlation with Malignant Tumors. |
| (4-1BB) | Am J Clin Pathol 115(4)543-549 (2001). |
| | Melero I et al. Monoclonal antibodies against the 4-1BB T-cell activation molecule eradicate established |
| | tumors. Nat Med 3: 682-5 (1997) (abstract). |
| | Niu L et al. Cytokine-mediated disruption of lymphocyte trafficking, hemopoiesis, and induction of |
| | lymphopenia, anemia, and thrombocytopenia in anti-CD137-treated mice. J Immunol. 178(7): 4194-4213 (2007). |
| | Palazon A et al. Agonist anti-CD137 mAb act on tumor endothelial cells to enhance recruitment of activated T |
| | lymphocytes. Cancer Res. 71(3): 801-11 (February 2011). |
| CXCR4 | Akashi-T et al. Chemokine receptor CXCR4 expression and prognosis in patients with metastatic |
| | prostate cancer. Cancer Sci 99(3): 539-542 (2008). |
| | Mirisola-V. et al. CXCL12/SDF1 expression by breast cancers is an independent prognostic marker of |
| | disease-free and overall surviva., Eur J Cancer 45(14): 2579-87 (2009) (abstract). |
| | Gassmann P et al. CXCR4 regulates the early extravasation of metastatic tumor cells in vivo. |
| | Neoplasia. 11(7): 651-61. (2009). |
| | Roland Jet al. Role of the intracellular domains of CXCR4 in SDF-1—mediated signaling. Blood. 101: |
| | 399-406 (2003). |
| | Fischer T et al. Reassessment of CXCR4 chemokine receptor expression in human normal and |
| | neoplastic tissues using the novel rabbit monoclonal antibody UMB-2. PLoS One. 3(12): e4069 (2008). |
| | Otsuka S and Bebb G. The CXCR4/SDF-1 Chemokine Receptor Axis. J Thorac Oncol. 3: 1379-1383 (2008). |
| | Xu C et al. Human anti-CXCR4 antibodies undergo VH replacement, exhibit functional V-region sulfation, |
| | and define CXCR4 antigenic heterogeneity. J Immunol 179(4): 2408-2418 (2007). |
| ACVRL1/ | Goff L et al. Phase I study of pf-03446962, a fully human mab against alk 1, a TGFbeta receptor involved |
| ALK1 | in tumor angiogenesis J Clin Oncol 28(15 suppl): 3034 (2010) (abstract). |
| | Hu-Lowe D D et al. Targeting activin receptor-like kinase 1 inhibits angiogenesis and tumorigenesis through |
| | a mechanism of action complementary to anti-VEGF therapies. Cancer Res; 71: 1362-73 (2011). |
| | Mancuso P, et al. Validation of a standardized method for enumerating circulating endothelial cells and |
| | progenitors: flow cytometry and molecular and ultrastructural analyses Clin Cancer Res 15: 267-73 (2009). |
| | Naeem S et al. Bone marrow involvement in systemic ALK+ anaplastic large cell lymphoma: morphological |
| | resemblance with Hodgkin's lymphoma. J Coll Physicians Surg Pak 16(2): 148-9 (2006) (abstract). |
| PD-1 | Iwai Y et al. Involvement of PD-L1 on tumor cells in the escape from host immune system and tumor |
| | immunotherapy by PD-L1 blockade. Proc Natl Acad Sci 19(19): 12293-12297 (2002). |
| | Toshiro I et al. Analysis of the Role of Negative T Cell Costimulatory Pathways in CD4 and CD8 T Cell- |
| | Mediated Alloimmune Responses In Vivo. JImmunol, 174: 6648-6656 (2005). |
| | Brahmer JR et al. Phase I study of single-agent anti-programmed death-1 (MDX-1106) in refractory solid |
| | tumors: safety, clinical activity, pharmacodynamics, and immunologic correlates. J Clin Oncol 28: 3167-3175 |
| | (2010). |
| | Tsushima F et al. Interaction between B7-H1 and PD-1 Determines Initiation and Reversal of T-Cell Anergy. |
| | Blood 110(10): 180-185 (2007). |
| PD-L1 | Blank C et al. Blockade of PD-L1 (B7-H1) augments human tumor-specific T cell responses in vitro. |
| | Int J Cancer 119: 317-327 (2006) (abstract). |
| | Ishida M et al. Differential expression of PD-L1 and PD-L2, ligands for an inhibitory receptor PD-1, in the |
| | cells of lymphohematopoietic tissues. Immunol Lett 84(1): 57-62 (2002) (abstract). |
| | Thompson Hit et al. Tumor B7-H1 Is Associated with Poor Prognosis in Renal Cell Carcinoma Patients with |
| | Long-term Follow-up. Cancer Res 66(7): 3381-3385 (2006). |
| | Latchman YE et al. PD-L1-deficient mice show that PD-L1 on T cells, antigen-presenting cells, and h |
| | ost tissues negative lyregulates T cells. Proc Natl Acad Sci 101(29): 10691-10696 (2004). |
| | Dong H et al. Costimulating aberrant T cell responses by B7-H1 autoantibodies in rheumatoid arthritis. |
| | J Clin Invest 111: 363-370 (2003), |
| | Brahmer J R et al. Phase I study of single-agent anti-programmed death-1 (MDX-1106) in refractory solid |
| | tumors: safety, clinical activity, pharmacodynamics, and immunologic correlates. J Clin Oncol 28: |
| | 3167-3175 (2010). |
| | Hamanishi J et al. Programmed cell death 1 ligand 1 and tumor infiltrating CD8 T lymphocytes are |
| | prognostic factors of human ovarian cancer. Proc Natl Acad Sci 105(9): 3360-65 (2007). |
| CD70 | Israel BF et al. Anti-CD70 antibodies: a potential treatment for EBV+ CD70-expressing lymphoma., |
| | Mol Cancer Ther 4(12): 2037-2044 (2005). |
| | Lens S M et al. Aberrant expression and reverse signalling of CD70 on malignant B cells. Br J |
| | Haematol 106: 491-503 (1999). |
| | Ranheim E A et al, Expression of CD27 and its ligand, CD70, on chronic lymphocytic leukemia B |
| | cells. Blood 85: 3556-65 (1995). |
| | Zambello R et al. Analysis of TNF-receptor and ligand superfamily molecules in patients with |
| | lymphoproliferative disease of granular lymphocytes. Blood 96: 647-54 (2000). |
| | Bullock TN et al. Induction of CD70 on dendritic cells through CD40 or TLR stimulation contributes to |
| | the development of CD8+ T cell responses in the absence of CD4+ T cells. J Immunol 174: 710-7 (2005). |
| CD74 | Stein Ret al. CD74: A New Candidate Target for the Immunotherapy of B-Cell Neoplasms Clin |
| | Cancer Res 13(18): 5556s-5563s (2007). |
| | Starlets D et al. Cell surface CD74 initiates a signaling cascade leading to cell proliferation and |
| | survival. Blood 107: 4807-16 (2006). |
| | Stein R et al. Anti-proliferative activity of a humanized anti-CD74 monoclonal antibody, hLL1, on |
| | B-cell malignancies. Blood 104: 3705-11 (2004). |
| | Chang C H et al. Effective therapy of human lymphoma xenografts with a novel recombinant |
| | ribonuclease/anti-CD74 humanized IgG4 antibody immunotoxin. Blood 106: 4308-14 (2005). |
| | Burton J D et al. CD74 Is Expressed by Multiple Myeloma and Is a Promising Target for Therapy. |
| | Clin Cancer Res 10(19): 6606-6611 (2004). |
| CD56 | Fossella V et al. Phase II trial of BB-10901 (huN901-DM1) given weekly for four consecutive weeks |
| | every 6 weeks in patients with relapsed SCLC and CD56-positive small cell carcinoma. |
| | J Clin Oncol 23(16 suppl): 7159-7159 (2005) (abstract). |
| | Roguska M A et al. Humanization of murine monoclonal antibodies through variable domain resurfacing. |
| | Proc Natl Acad Sci 91(3): 969-73 (1994). |
| | Cooper MA et al. Human natural killer cells: a unique innate immunoregulatory role for the CD56(bright) |
| | subset. Blood 97(10): 3146-51 (2001). |
| | Campbell J J et al. Unique subpopulations of CD56+ NK and NK-T peripheral blood lymphocytes |
| | identified by chemokiner eceptor expression repertoire. J Immunol 166(11): 6477-82 (2001). |
| | De Maria A et al. Revisiting human natural killer cell subset function revealed cytolytic CD56(dim)CD16+ |
| | rNK cells as apid producers of abundant IFN-gamma on activation. Proc Natl Acad Sci 108: 728-32 (2011). |
| | Cho E Y et al. Immunohistochemical study of the expression of adhesion molecules in ovarian serous |
| | neoplasms. Pathol Int 56(2): 62-70 (2006) (abstract). |
| CD40 | Luqman M et al. The antileukemia activity of a human anti-CD40 antagonist antibody, HCD122, on |
| | human chronic lymphocytic leukemia cells Blood 112(3): 711-720 (2008). |
| | Uckum F M et al. Temporal association of CD40 antigen expression with discrete stages of human B-cell |
| | ontogeny and the efficacy of anti-CD40 immunotoxins against clonogenic B-lineage acute lymphoblastic |
| | leukemia as well as B- lineage non-Hodgkin's lymphoma cells Blood 76 (12) 2449-2456 (1990). |
| | Vyth-Dreese FA et al. Localization in situ of costimulatory molecules and cytokines in B-cell non- |
| | Hodgkin's lymphoma. Immunology 94: 580-586 (1998). |
| | Hulkkonen J et al. Surface antigen expression in chronic lymphocytic leukemia: clustering analysis, |
| | interrelationships and effects of chromosomal abnormalities. Leukemia 16: 178-185 (2002). |
| | Kater A P et al. CD40 stimulation of B-cell chronic lymphocytic leukaemia cells enhances the anti- |
| | apoptotic profile, but also Bid expression and cells remain susceptible to autologous cytotoxic T-lymphocyte |
| | attack. Br J Haematol 127: 404-415 (2004) (abstract). |
| | Melter M et al. Ligation of CD40 induces the expression of vascular endothelial growth factor by |
| | endothelial cells and monocytes and promotes angiogenesis in vivo. Blood 96: 3801-3808 (2000). |
| CD19 | Blanc V et al. 5AR3419: An Anti-CD19-Maytansinoid Immunoconjugate for the Treatment of B-Cell |
| | Malignancies. Clin Cancer Res 17(20): 6448-6458 (2011). |
| | Herbst R et al. B-cell depletion in vitro and in vivo with an afucosylated anti-CD19 antibody. J Pharmacol |
| | Exp Ther335: 213-22 (2010). |
| | D'Arena G et al. Quantitative flow cytometry for the differential diagnosis of leukemic B-cell chronic |
| | lymphoproliferative disorders. Am J Hemat 64: 275-281 (2000) (abstract). |
| | Johnson N A et al. Diffuse large B-cell lymphoma: reduced CD20 expression is associated with an |
| | inferior survival. Blood 113: 3773-3780 (2009). |
| | Sato S et al. Altered blood B lymphocyte homeostasis in systemic sclerosis: expanded naive B cells |
| | and diminished but activated memory B cell. Arthritis Rheum 50: 1918-1927 (2004) (abstract). |
| | Kansas G S et al. Transmembrane signals generated through MHC class II, CD19, CD20, CD39, and |
| | CD40 antigens induce LFA-1-dependent and independent adhesion in human B cells through a tyrosine |
| | kinase-dependent pathway. J Immunol 147: 4094-4102 (1991) (abstract). |
| CD80 | Leonard J W et al. A phase I/II study of galiximab (an anti-CD80 monoclonal antibody) in combination |
| | with rituximab for relapsed or refractory, follicular lymphoma. Ann Oncol 18(7): 1216-1223 (2007). |
| | Vyth-Dreese F A et al. Localization in situ of costimulatory molecules and cytokines in B-cell non- |
| | Hodgkin's lymphoma. Immunology 94: 580-586 (1998). |
| | Dorfman D M et al. In vivo expression of B7-1 and B7-2 by follicular lymphoma cells can prevent induction |
| | of T-cell anergy but is insufficient to induce significant T-cell proliferation. Blood 90: 4297-4306 (1997). |
| | Dogan A et al. Follicular lymphomas contain a clonally linked but phenotypically distinct neoplastic B-cell |
| | population in the interfollicular zone Blood 91: 4708-4714 (1998). |
| | Suvas S et al. Distinct role of CD80 and CD86 in the regulation of the activation of B cell and B cell |
| | lymphoma. J Biol Chem 277: 7766-7775 (2002). |
| CD86 | Vincenti, F. What's in the pipeline? New immunosuppressive drugs in transplantation. Am J Transplant 2: |
| | 898-903 (2002) (abstract). |
| | Vyth-Dreese FA et al. Localization in situ of costimulatory molecules and cytokines in B-cell non- |
| | Hodgkin's lymphoma. Immunology 94: 580-586 (1998). |
| | Dorfman D M et al. In vivo expression of B7-1 and B7-2 by follicular lymphoma cells can prevent induction |
| | of T-cell anergy but is insufficient to induce significant T-cell proliferation. Blood 90: 4297-4306 (1997). |
| | Dogan A et al. Follicular lymphomas contain a clonally linked but phenotypically distinct neoplastic B-cell |
| | population in the interfollicular zone. Blood 91: 4708-4714 (1998). |
| | Suvas S et al. Distinct role of CD80 and CD86 in the regulation of the activation of B cell and B cell |
| | lymphoma. J Biol Chem 277: 7766-7775 (2002). |
| CD2 | Matthews J B et al. Clinical Trials of Transplant Tolerance: Slow But Steady Progress. Am J Transplant 3: |
| | 794-803 (2003). |
| | Przepiorka D et al. A phase II study of BTI-322, a monoclonal anti-CD2 antibody, for |
| | treatment of steroid-resistant acute graft-versus-host disease. Blood 92: 4066-4071 (1998). |
| | Latinne D et al. An anti-CD2 mAb induces immunosuppression and hyporesponsiveness of CD2+ human |
| | T cells in vitro. Int Immunol 8: 1113 (1996) (abstract). |
| | Guckel B et. Anti-CD2 antibodies induce T cell unresponsiveness in vivo. J Exp Med 174:957, (1991). |
| | Bromberg J S et al. Anti-CD2 monoclonal antibodies alter cell-mediated immunity in vivo. |
| | (abstract). Transplantation 51: 219 (1991) |
| CD30 | Maeda-N. Susceptibility of human T-cell leukemia virus type I-infected cells to humanized anti-CD30 |
| | monoclonal antibodies in vitro and in vivo. Cancer Sci 101(1): 224-30 (2010) (epub 2009 Sep. 8) (abstract). |
| | Schlapschy M et al. Functional humanization of an anti-CD30 Fab fragment for the immunotherapy of |
| | Hodgkin's lymphoma using an in vitro evolution approach. Protein Eng Des Sel 17(12): 847-860 (2004). |
| | da Costa L et al. Immunoscintigraphy in Hodgkin's disease and anaplastic large cell lymphomas: results |
| | in 18 patients using the iodine radiolabeled monoclonal antibody FIRS-3. Ann Oncol. Sep; 3 Suppl 4: |
| | 53-7 (1992) (abstract). |
| | Su C C et al. CD30 Is Involved in Inhibition of T-Cell Proliferation by Hodgkin's Reed-Sternberg Cells, |
| | Cancer Res 64(6): 2148-2152 (2004). |
| | Pinto A et al. Human eosinophils express functional CD30 ligand and stimulate proliferation of a |
| | Hodgkin's disease cell line. Blood 88 (9) 3299-3305 (1996). |
| | Barth-S et al. Ki-4(scFv)—ETA', a new recombinant anti-CD30 immunotoxin with highly specific |
| | cytotoxic activity against disseminated Hodgkin tumors in SCID mice. Blood 95 (12): 3909-3914 (2000). |
| CD20 | McLaughlin P et al. Rituximab chimeric anti-CD20 monoclonal antibody therapy for relapsed indolent |
| | lymphoma: half of patients respond to a four-dose treatment program. J Clin Oncol 16: 2825-33 (1998) |
| | (abstract). |
| | Kaminski M S et al. Radioimmunotherapy with iodine (131)I tositumomab for relapsed or refractory |
| | B-cell non-Hodgkinlymphoma: updated results and long-term follow-up of the University of |
| | Michigan experience. Blood 96: 1259-66 (2000). |
| | Coiffier B et al. Rituximab in combination with CHOP improves survival in elderly patients with |
| | aggressive non-Hodgkin's lymphoma. Semin Oncol 29(2 Suppl 6): 18-22 (2002) (abstract). |
| | Witzig T E et al. Randomized controlled trial of yttrium-90-labeled ibritumomab tiuxetan |
| | radioimmunotherapy versus rituximab immunotherapy for patients with relapsed or refractory low- |
| | grade, follicular, or transformed B-cell non-Hodgkin's lymphoma. J Clin Oncol 20: 2453-6 (2003) |
| | (abstract). |
| | Maddipatla-S et al. Augmented Antitumor Activity against B-Cell Lymphoma by a Combination of |
| | Monoclonal Antibodies Targeting TRAIL-R1 and CD20. Clin Cancer Res 13(15): 4556-4564 (2007). |
| CD33 | Sievers E L et al. Selective ablation of acute myeloid leukemia using antibody-targeted chemotherapy: a |
| | phase I study of an anti-CD33 calicheamicin immunoconjugate. Blood 93: 3678-84 (1999). |
| | Hauswirth A W et al. The Target Receptor Siglec-3 (CD33) Is Expressed on AML Stem Cells in a |
| | Majority of All Patients with AML Blood 106 (11): 4324 (2005) (abstract). |
| | Caron PC et al. Biological and Immunological Features of Humanized M195 (Anti-CD33) Monoclonal |
| | Antibodies. Cancer Res 52(24): 6761-6767 (1992). |
| | Stiff P J et al. Anti-CD33 monoclonal antibody and etoposide/cytosine arabinoside combinations |
| | for the ex vivo purification of bone marrow in acute nonlymphocytic leukemia. Blood 77 (2): |
| | 355-362 (1991). |
| | Roy D C et al. Anti-MY9-blocked-ricin: an immunotoxin for selective targeting of acute myeloid |
| | leukemia cells. Blood 77 (11): 2404-2412 (1991). |
| CD22 | Carnahan J et al. Epratuzumab, a humanized monoclonal antibody targeting CD22: characterization |
| | of in vitro properties. Clin Cancer Res 9(10 Pt 2):39825-905 (2003) (abstract). |
| | Kreitman R J et al. Efficacy of the anti-CD22 recombinant immunotoxin BL22 in chemotherapy- |
| | resistant hairy-cell leukemia. N Engl J Med 345: 241-47 (2001). |
| | Robbins B A et al. Diagnostic application of two-color flow cytometry in 161 cases of hairy cell |
| | leukemia. Blood 82: 1277-87 (1993). |
| | Cordone I et al. Diagnostic relevance of peripheral blood Immunocytochemistry in hairy cell |
| | leukemia. J Clin Pathol 48: 955-960 (1995). |
| | Amlot P L et al. A phase I study of an anti-CD22-deglycosylated ricin A chain immunotoxin in the |
| | treatment of B-cell lymphomas resistant to conventional therapy. Blood 82: 2624-2633 (1993). |

The FDA maintains listings of approved antibody drugs for treating cancer, many of which bind to cancer antigens and can be employed in this context. See The Orange Book Online or Drugs@FDA on the FDA website. The FDA also maintains listings of clinical trials in progress in the clinicaltrials.gov database, which may be searched by disease names. Table 3D provides a representative list of approved antibodies with specificity for tumor cells. Table 3E provides a representative list of antibodies in development with specificity for tumor cells.

TABLE 3D
Representative antibodies approved for cancer indications

| International | | 1st indication |
|---|---|---|
| Nonproprietary | | approved/ |
| Name | Target; Format | reviewed |
| Ado- | HER2; Humanized IgG1, | Breast cancer |
| trastuzumab | ADC | |
| emtansine | | |
| Alemtuzumab | CD52; Humanized IgG1 | Chronic myeloid |
| | | leukemia; |
| | | multiple sclerosis |
| Atezolizumab | PD-L1 Humanized IgG1 | Bladder cancer |
| Avelumab | PD-L1; Human IgG1 | Merkel cell carcinoma |
| Bevacizumab | VEGF; Humanized IgG1 | Colorectal cancer |
| Blinatumomab | CD19, CD3; Murine | Acute lymphoblastic |
| | bispecific tandem scFv | leukemia |
| Brentuximab | CD30; Chimeric IgG1, | Hodgkin lymphoma, |
| vedotin | ADC | systemic anaplastic |
| | | large cell lymphoma |
| Catumaxomab | EPCAM/CD3; Rat/mouse | Malignant ascites |
| | bispecific mAb | |
| Cemiplimab | PD-1; Human mAb | Cutaneous squamous |
| | | cell carcinoma |
| Cetuximab | EGFR; Chimeric IgG1 | Colorectal cancer |
| Daratumumab | CD38; Human IgG1 | Multiple myeloma |
| Dinutuximab | GD2; Chimeric IgG1 | Neuroblastoma |
| Durvalumab | PD-L1; Human IgG1 | Bladder cancer |
| Edrecolomab | EpCAM; Murine IgG2a | Colorectal cancer |
| Elotuzumab | SLAMF7; Humanized IgG1 | Multiple myeloma |
| Gemtuzumab | CD33; Humanized IgG4, | Acute myeloid |
| | ADC | leukemia |
| Ibritumomab | CD20; Murine IgG1 | Non-Hodgkin |
| tiuxetan | | lymphoma |
| Inotuzumab | CD22; Humanized IgG4, | Hematological |
| | ADC | malignancy |
| Ipilimumab | CTLA-4; Human IgG1 | Metastatic melanoma |
| Mogamuizumab | CCR4; Humanized IgG1 | Cutaneous T-cell |
| | | lymphoma |
| Moxetumomab | CD22; Murine IgG1 dsFy | Hairy cell leukemia |
| pasudotox | immunotoxin | |
| Necitumumab | EGFR; Human IgG1 | Non-small cell lung |
| | | cancer |
| Nivolumab | PD-1; Human IgG4 | Melanoma, non-small |
| | | cell lung cancer |
| Obinutuzumab | CD20; Humanized IgGl; | Chronic lymphocytic |
| | Glycoengineered | leukemia |
| Ofatumumab | CD20; Human IgG1 | Chronic lymphocytic |
| | | leukemia |
| Olaratumab | PDGRFα; Human IgG1 | Soft tissue sarcoma |
| Panitumumab | EGFR; Human IgG2 | Colorectal cancer |
| Pembrolizumab | PD-1; Humanized IgG4 | Melanoma |
| Pertuzumab | HER2; Humanized IgG1 | Breast Cancer |
| Ramucirumab | VEGFR2; Human IgG1 | Gastric cancer |
| Rituximab | CD20; Chimeric IgG1 | Non-Hodgkin lymphoma |
| Sacituzumab | TROP-2; Humanized IgG1 | Triple-negative |
| govitecan | ADC | breast cancer |
| Tositumomab- | CD20; Murine IgG2a | Non-Hodgkin lymphoma |
| I131 | | |
| Trastuzumab | HER2; Humanized IgG1 | Breast cancer |

TABLE 3E
Antibodies in development for cancer indications

| INN or code | Molecular | | Late-stage |
|---|---|---|---|
| name | format | Target | study indication(s) |
| Utomilumab | Human IgG2 | CD137 | Diffuse large B-cell |
| | | (4-1BB) | lymphoma |
| XMAB-5574, | Humanized | CD19 | Diffuse large B-cell |
| MOR208 | IgG1 | | lymphoma |
| Ublituximab | Chimeric IgG1 | CD20 | Chronic |
| | | | lymphocytic |
| | | | Leukemia, non- |
| | | | Hodgkin |
| | | | lymphoma, multiple |
| | | | sclerosis |
| Moxetumomab | Murine IgG1 | CD22 | Hairy cell leukemia |
| pasudotox | dsFy | | |
| | immunotoxin | | |
| Isatuximab | Humanized | CD38 | Multiple myeloma |
| | IgG1 | | |
| Polatuzumab | Humanized | CD79b | Diffuse large B-cell |
| vedotin | IgG1 ADC | | lymphoma |
| Tremelimumab | Human IgG2 | CTLA-4 | Non-small cell lung, |
| | | | head & neck, |
| | | | urothelial cancer, |
| | | | hepatocellular |
| | | | carcinoma |
| Rovalpituzumab | Humanized | DLL3 | Small cell lung |
| tesirine | IgG1 ADC | | cancer |
| Depatuxizumab | IgG1 ADC | EGFR | Glioblastoma |
| mafodotin | | | |
| Carotuximab | Chimeric IgG1 | Endoglin | Soft tissue sarcoma, |
| | | | angiosarcoma, |
| | | | renal cell carcinoma, |
| | | | wet age- |
| | | | related macular |
| | | | degeneration |
| Oportuzumab | Humanized | EpCAM | Bladder cancer |
| monatox | scEv | | |
| | immunotoxin | | |
| L19IL2/ | scEv immuno- | Fibronectin | Melanoma |
| L19TNE | conjugates | extra- | |
| | | domain B | |
| Mirvetuximab | IgG1 ADC | Folate | Epithelial ovarian |
| soravtansine | | receptor 1 | cancer, peritoneal |
| | | | carcinoma, |
| | | | fallopian tube cancer |
| Glembatumumab | Human IgG2 | gpNMB | gpNMB+ breast |
| vedotin | ADC | | cancer, melanoma |
| Margetuximab | Chimeric IgG1 | HER2 | Breast cancer |
| (vic-) | Humanized | HER2 | Breast cancer |
| trastuzumab | IgG1 ADC | | |
| duocarmazine | | | |
| DS-8201 | Humanized | HER2 | HER2+ gastric or |
| | ADC | | gastroesophageal |
| | | | junction |
| | | | adenocarcinoma |
| Andecaliximab | Humanized | MMP-9 | Gastric cancer or |
| | IgG4 | | gastroesophageal |
| | | | junction |
| | | | adenocarcinoma |
| Racotumomab | Murine IgG1 | NGcGM3 | Non-small cell |
| | | | lung cancer |
| Camrelizumab | Humanized | PD-1 | Hepatocellular |
| | IgG4 | | carcinoma, |
| | | | esophageal |
| | | | carcinoma |
| Cemiplimab | Human mAb | PD-1 | Cutaneous |
| | | | squamous cell |
| | | | carcinoma; non- |
| | | | small cell lung |
| | | | cancer, |
| | | | cervical cancer |
| IBI308 | Human mAb | PD-1 | Squamous cell |
| | | | non-small cell |
| | | | lung cancer |
| BGB-A317 | Humanized | PD-1 | Non-small cell lung |
| | mAb | | cancer |
| BCD-100 | Human mAb | PD-1 | Melanoma |
| PDR001 | Humanized | PD-1 | Melanoma |
| | IgG4 | | |
| Sacituzumab | IgG1 ADC | TROP-2 | Triple-neg. breast |
| govitecan | | (epithelial | cancer |
| | | glyco- | |
| | | protein-1) | |

Other antibodies well-known in the art may be used as targeting moieties to target to a given cancer. The antibodies and their respective antigens include nivolumab (anti-PD-1 Ab), TA99 (anti-gp75), 3F8 (anti-GD2), 8H9 (anti-B7-H3), abagovomab (anti-CA-125 (imitation)), adecatumumab (anti-EpCAM), afutuzumab (anti-CD20), alacizumab pegol (anti-VEGFR2), altumomab pentetate (anti-CEA), amatuximab (anti-mesothelin), AME-133 (anti-CD20), anatumomab mafenatox (anti-TAG-72), apolizumab (anti-HLA-DR), arcitumomab (anti-CEA), bavituximab (anti-phosphatidylserine), bectumomab (anti-CD22), belimumab (anti-BAFF), besilesomab (anti-CEA-related antigen), bevacizumab (anti-VEGF-A), bivatuzumab mertansine (anti-CD44 v6), blinatumomab (anti-CD19), BMS-663513 (anti-CD137), brentuximab vedotin (anti-CD30 (TNFRSF8)), cantuzumab mertansine (anti-mucin CanAg), cantuzumab ravtansine (anti-MUC1), capromab pendetide (anti-prostatic carcinoma cells), carlumab (anti-MCP-1), catumaxomab (anti-EpCAM, CD3), cBR96-doxorubicin immunoconjugate (anti-Lewis-Y antigen), CC49 (anti-TAG-72), cedelizumab (anti-CD4), Ch.14.18 (anti-GD2), ch-TNT (anti-DNA associated antigens), citatuzumab bogatox (anti-EpCAM), cixutumumab (anti-IGF-1 receptor), clivatuzumab tetraxetan (anti-MUC1), conatumumab (anti-TRAIL-R2), CP-870893 (anti-CD40), dacetuzumab (anti-CD40), daclizumab (anti-CD25), dalotuzumab (anti-insulin-like growth factor I receptor), daratumumab (anti-CD38 (cyclic ADP ribose hydrolase)), demcizumab (anti-DLL4), detumomab (anti-B-lymphoma cell), drozitumab (anti-DR5), duligotumab (anti-HER3), dusigitumab (anti-ILGF2), ecromeximab (anti-GD3 ganglioside), edrecolomab (anti-EpCAM), elotuzumab (anti-SLAMF7), elsilimomab (anti-IL-6), enavatuzumab (anti-TWEAK receptor), enoticumab (anti-DLL4), ensituximab (anti-5AC), epitumomab cituxetan (anti-episialin), epratuzumab (anti-CD22), ertumaxomab (anti-HER2/neu, CD3), etaracizumab (anti-integrin αvβ3), faralimomab (anti-Interferon receptor), farletuzumab (anti-folate receptor 1), FBTA05 (anti-CD20), ficlatuzumab (anti-HGF), figitumumab (anti-IGF-1 receptor), flanvotumab (anti-TYRP1(glycoprotein 75)), fresolimumab (anti-TGF β), futuximab (anti-EGFR), galiximab (anti-CD80), ganitumab (anti-IGF-I), gemtuzumab ozogamicin (anti-CD33), girentuximab (anti-carbonic anhydrase 9 (CA-IX)), glembatumumab vedotin (anti-GPNMB), guselkumab (anti-IL13), ibalizumab (anti-CD4), ibritumomab tiuxetan (anti-CD20), icrucumab (anti-VEGFR-1), igovomab (anti-CA-125), IMAB362 (anti-CLDN18.2), IMC-CS4 (anti-CSF1R), IMC-TR1 (TGFβRII), imgatuzumab (anti-EGFR), inclacumab (anti-selectin P), indatuximab ravtansine (anti-SDC1), inotuzumab ozogamicin (anti-CD22), intetumumab (anti-CD51), ipilimumab (anti-CD152), iratumumab (anti-CD30 (TNFRSF8)), KM3065 (anti-CD20), KW-0761 (anti-CD194), LY2875358 (anti-MET) labetuzumab (anti-CEA), lambrolizumab (anti-PDCD1), lexatumumab (anti-TRAIL-R2), lintuzumab (anti-CD33), lirilumab (anti-KIR2D), lorvotuzumab mertansine (anti-CD56), lucatumumab (anti-CD40), lumiliximab (anti-CD23 (IgE receptor)), mapatumumab (anti-TRAIL-R1), margetuximab (anti-ch4D5), matuzumab (anti-EGFR), mavrilimumab (anti-GMCSF receptor a-chain), milatuzumab (anti-CD74), minretumomab (anti-TAG-72), mitumomab (anti-GD3 ganglioside), mogamulizumab (anti-CCR4), moxetumomab pasudotox (anti-CD22), nacolomab tafenatox (anti-C242 antigen), naptumomab estafenatox (anti-5T4), narnatumab (anti-RON), necitumumab (anti-EGFR), nesvacumab (anti-angiopoietin 2), nimotuzumab (anti-EGFR), nivolumab (anti-IgG4), nofetumomab merpentan, ocrelizumab (anti-CD20), ocaratuzumab (anti-CD20), olaratumab (anti-PDGF-R α), onartuzumab (anti-c-MET), ontuxizumab (anti-TEM1), oportuzumab monatox (anti-EpCAM), oregovomab (anti-CA-125), otlertuzumab (anti-CD37), pankomab (anti-tumor specific glycosylation of MUC1), parsatuzumab (anti-EGFL7), pascolizumab (anti-IL-4), patritumab (anti-HER3), pemtumomab (anti-MUC1), pertuzumab (anti-HER2/neu), pidilizumab (anti-PD-1), pinatuzumab vedotin (anti-CD22), pintumomab (anti-adenocarcinoma antigen), polatuzumab vedotin (anti-CD79B), pritumumab (anti-vimentin), PRO131921 (anti-CD20), quilizumab (anti-IGHE), racotumomab (anti-N-glycolylneuraminic acid), radretumab (anti-fibronectin extra domain-B), ramucirumab (anti-VEGFR2), rilotumumab (anti-HGF), robatumumab (anti-IGF-1 receptor), roledumab (anti-RHD), rovelizumab (anti-CD11 & CD18), samalizumab (anti-CD200), satumomab pendetide (anti-TAG-72), seribantumab (anti-ERBB3), SGN-CD19A (anti-CD19), SGN-CD33A (anti-CD33), sibrotuzumab (anti-FAP), siltuximab (anti-IL-6), solitomab (anti-EpCAM), sontuzumab (anti-episialin), tabalumab (anti-BAFF), tacatuzumab tetraxetan (anti-alpha-fetoprotein), taplitumomab paptox (anti-CD19), telimomab aritox, tenatumomab (anti-tenascin C), teneliximab (anti-CD40), teprotumumab (anti-CD221), TGN1412 (anti-CD28), ticilimumab (anti-CTLA-4), tigatuzumab (anti-TRAIL-R2), TNX-650 (anti-IL-13), tositumomab (anti-CS20), tovetumab (anti-CD140a), TRBS07 (anti-GD2), tregalizumab (anti-CD4), tremelimumab (anti-CTLA-4), TRU-016 (anti-CD37), tucotuzumab celmoleukin (anti-EpCAM), ublituximab (anti-CD20), urelumab (anti-4-1BB), vantictumab (anti-Frizzled receptor), vapaliximab (anti-AOC3 (VAP-1)), vatelizumab (anti-ITGA2), veltuzumab (anti-CD20), vesencumab (anti-NRP1), visilizumab (anti-CD3), volociximab (anti-integrin α5β1), vorsetuzumab mafodotin (anti-CD70), votumumab (anti-tumor antigen CTAA16.88), zalutumumab (anti-EGFR), zanolimumab (anti-CD4), zatuximab (anti-HER1), ziralimumab (anti-CD147 (basigin)), RG7636 (anti-ETBR), RG7458 (anti-MUC16), RG7599 (anti-NaPi2b), MPDL3280A (anti-PD-L1), RG7450 (anti-STEAP1), and GDC-0199 (anti-Bcl-2).

Antibodies that bind these antigens may also be used as targeting moieties, especially for the types of cancers noted: aminopeptidase N (CD13), annexin A1, B7-H3 (CD276, various cancers), CA125 (ovarian cancers), CA15-3 (carcinomas), CA19-9 (carcinomas), L6 (carcinomas), Lewis Y (carcinomas), Lewis X (carcinomas), alpha fetoprotein (carcinomas), CA242 (colorectal cancers), placental alkaline phosphatase (carcinomas), prostate s7pecific antigen (prostate), prostatic acid phosphatase (prostate), epidermal growth factor (carcinomas), CD2 (Hodgkin's disease, NHL lymphoma, multiple myeloma), CD3 epsilon (T-cell lymphoma, lung, breast, gastric, ovarian cancers, autoimmune diseases, malignant ascites), CD19 (B cell malignancies), CD20 (non-Hodgkin's lymphoma, B-cell neoplasmas, autoimmune diseases), CD21 (B-cell lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (metastatic melanoma, sarcoma), CD52 (leukemia), CD56 (small cell lung cancers, ovarian cancer, Merkel cell carcinoma, and the liquid tumor, multiple myeloma), CD66e (carcinomas), CD70 (metastatic renal cell carcinoma and non-Hodgkin lymphoma), CD74 (multiple myeloma), CD80 (lymphoma), CD98 (carcinomas), CD123 (leukemia), mucin (carcinomas), CD221 (solid tumors), CD22 (breast, ovarian cancers), CD262 (NSCLC and other cancers), CD309 (ovarian cancers), CD326 (solid tumors), CEACAM3 (colorectal, gastric cancers), CEACAM5 (CEA, CD66e) (breast, colorectal and lung cancers), DLL4 (A-like-4), EGFR (various cancers), CTLA4 (melanoma), CXCR4 (CD 184, heme-oncology, solid tumors), Endoglin (CD 105, solid tumors), EPCAM (epithelial cell adhesion molecule, bladder, head, neck, colon, NHL prostate, and ovarian cancers), ERBB2 (lung, breast, prostate cancers), FCGR1 (autoimmune diseases), FOLR (folate receptor, ovarian cancers), FGFR (carcinomas), GD2 ganglioside (carcinomas), G-28 (a cell surface antigen glycolipid, melanoma), GD3 idiotype (carcinomas), heat shock proteins (carcinomas), HER1 (lung, stomach cancers), HER2 (breast, lung and ovarian cancers), HLA-DR10 (NHL), HLA-DRB (NHL, B cell leukemia), human chorionic gonadotropin (carcinomas), IGF1R (solid tumors, blood cancers), IL-2 receptor (T-cell leukemia and lymphomas), IL-6R (multiple myeloma, RA, Castleman's disease, IL6 dependent tumors), integrins (αvβ3, α5β1, α6β4, α11β3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 (breast, ovarian, cervix, bronchus and gastrointestinal cancer), MUC16 (CA125) (ovarian cancers), CEA (colorectal cancer), gp100 (melanoma), MARTI (melanoma), MPG (melanoma), MS4A1 (membrane-spanning 4-domains subfamily A, small cell lung cancers, NHL), nucleolin, Neu oncogene product (carcinomas), P21 (carcinomas), nectin-4 (carcinomas), paratope of anti-(N-glycolylneuraminic acid, breast, melanoma cancers), PLAP-like testicular alkaline phosphatase (ovarian, testicular cancers), PSMA (prostate tumors), PSA (prostate), ROB04, TAG 72 (tumour associated glycoprotein 72, AML, gastric, colorectal, ovarian cancers), T-cell transmembrane protein (cancers), Tie (CD202b), tissue factor, TNFRSF10B (tumor necrosis factor receptor superfamily member 10B, carcinomas), TNFRSF13B (tumor necrosis factor receptor superfamily member 13B, multiple myeloma, NHL, other cancers, RA and SLE), TPBG (trophoblast glycoprotein, renal cell carcinoma), TRAIL-R1 (tumor necrosis apoptosis inducing ligand receptor 1, lymphoma, NHL, colorectal, lung cancers), VCAM-1 (CD106, Melanoma), VEGF, VEGF-A, VEGF-2 (CD309) (various cancers). Some other tumor associated antigen targets have been reviewed (Gerber, et al, mAbs 2009 1:247-253; Novellino et al, Cancer Immunol Immunother. 2005 54:187-207, Franke, et al, Cancer Biother Radiopharm. 2000, 15:459-76, Guo, et al., Adv Cancer Res. 2013; 119: 421-475, Parmiani et al. J Immunol. 2007 178:1975-9). Examples of these antigens include Cluster of Differentiations (CD4, CDS5, CD6, CD7, CD8, CD9, CD10, CD11a, CD11b, CD11c, CD12w, CD14, CD15, CD16, CDw17, CD18, CD21, CD23, CD24, CD25, CD26, CD27, CD28, CD29, CD31, CD32, CD34, CD35, CD36, CD37, CD41, CD42, CD43, CD44, CD45, CD46, CD47, CD48, CD49b, CD49c, CD53, CD54, CD55, CD58, CD59, CD61, CD62E, CD62L, CD62P, CD63, CD68, CD69, CD71, CD72, CD79, CD81, CD82, CD83, CD86, CD87, CD88, CD89, CD90, CD91, CD95, CD96, CD100, CD103, CD105, CD106, CD109, CD117, CD120, CD127, CD133, CD134, CD135, CD138, CD141, CD142, CD143, CD144, CD147, CD151, CD152, CD154, CD156, CD158, CD163, CD166, CD168, CD184, CDw186, CD195, CD202 (a, b), CD209, CD235a, CD271, CD303, CD304), annexin A1, nucleolin, endoglin (CD105), ROB04, amino-peptidase N, -like-4 (DLL4), VEGFR-2 (CD309), CXCR4 (CD184), Tie2, B7-H3, WT1, MUC1, LMP2, HPV E6 E7, EGFRvIII, HER-2/neu, idiotype, MAGE A3, p53 nonmutant, NY-ESO-1, GD2, CEA, MelanA/MART1, Ras mutant, gp100, p53 mutant, proteinase3 (PR1), bcr-abl, tyrosinase, survivin, hTERT, sarcoma translocation breakpoints, EphA2, PAP, ML-IAP, AFP, EpCAM, ERG (TMPRSS2 ETS fusion gene), NA17, PAX3, ALK, androgen receptor, cyclin B 1, polysialic acid, MYCN, RhoC, TRP-2, GD3, fucosyl GM1, mesothelin, PSCA, MAGE A1, sLe(a), CYPIB I, PLAC1, GM3, BORIS, Tn, GloboH, ETV6-AML, NY-BR-1, RGS5, SART3, STn, carbonic anhydrase IX, PAXS, OY-TES1, sperm protein 17, LCK, HMWMAA, AKAP-4, SSX2, XAGE 1, B7H3, legumain, Tie 2, Page4, VEGFR2, MAD-CT-1, FAP, PDGFR-β, MAD-CT-2, and Fos-related antigen 1.

In some embodiments, the targeting moiety capable of targeting a cancer is not an antibody, but is another type of targeting moiety. A wide range of targeting moieties capable of targeting cancer are known, including DNA aptamers, RNA aptamers, albumins, lipocalins, fibronectins, ankyrins, CH1/2/3 scaffolds (including abdurins (IgG CH2 scaffolds)), fynomers, Obodies, DARPins, knotins, avimers, atrimers, anticallins, affilins, affibodies, bicyclic peptides, cys-knots, FN3 (adnectins, centryrins, pronectins, TN3), and Kunitz domains. These and other non-antibody scaffold structures may be used for targeting to a cancer cell. Smaller non-antibody scaffolds are rapidly removed from the bloodstream and have a shorter half-life than monocolonal antibodies. They also show faster tissue penetration owing to fast extravasation from the capillary lumen through the vascular endothelium and basement membrane. See Vazquez-Lombardi et al., Drug Discovery Today 20(1):1271-1283 (2015). A number of non-antibody scaffolds targeting cancer are already under clinical development, with other candicates in the preclinical stage. See Vazquez-Lombardi, Table 1.

TABLE 4A
Non-Antibody Scaffolds and Corresponding Targets

| Scaffold | Demonstrated Targets |
|---|---|
| Adnectin | EGFR, IGF-1R |
| Affibodies | HER2, EGFR, IGF-1R, HER3 |
| Affinlins | CTLA-4 |
| Anticalins | CD137/HER2 (a bispecific) |
| Atrimers | DR4 |
| Avimers | IL6 (could be used in oncology to |
| | block growth) |
| Bicyclic peptides | HER2 |
| Cys-knots | NaV1.7 (proof of concept) |
| DARPins | VEGF-a, HER2, VEGF/HGF |
| | (bispecific) |
| Fynomers | HER2 |
| Pronectins | VEGFR2 |
| TN3 | TRAILR2 |

In another embodiment, a targeting moiety may be a binding partner for a protein known to be expressed on the cancer cell. Such expression levels may include overexpression. For example, the binding partners described in Table 4 may bind to the following targets on a cancer cell:

TABLE 4B
Non-Antibody Binding Partners and Corresponding Targets

| Binding Partner | Target on Cancer Cell |
|---|---|
| IL-2 | IL-2 receptor |
| IL-4 | IL-4 receptor |
| IL-6 | IL-6 receptor |
| α-MSH | MSH receptor (melanocyte |
| | stimulating hormone receptor) |
| Transferrin | TR (transferrin receptor) |
| Folic acid | FOLR (folate receptor 1) and/or |
| | FOLH1 (folate hydroxylase) |
| EGF and/or TGFα | EGFR (EGF receptor) |
| PD1 | PD-L1 and/or PD-L2 |
| IL13 | IL-13R (Glioblastoma) |
| Stem cell factor | CXCR4 |
| Insulin-like growth factor (IGF) | IGFR |
| CD40 | CD40L |

The binding partner need not comprise the full length or wildtype sequence for the binding partners listed in Table 4B. All that is required is that the binding partner bind to the target on the cancer cell and can thus include truncated forms, analogs, variants, and derivatives that are well known in the art.

Additionally, in some embodiments, the binding partner may be an aptamer that is capable of binding to a protein known to be expressed on the cancer cell. Aptamers that bind cancer cells, such as cancer cells, are well known and methods for designing them are known.

Cell-based SELEX systems may be used to select a panel of target cell-specific aptamers from a random candidate library. A ssDNA or ssRNA pool may be dissolved in binding buffer and denatured and then incubated with target cells. After washing the bound DNAs or RNAs may be eluted by heating and then incubated with negative cells (if desired), centrifuged, and the supernatant removed. The supernatant may be amplified by PCR with biotin labeled primers. The selected sense ssDNA or ssRNA may be separated from the antisense biotinylated strand using streptavidin coated beads. To increase affinity, washing strength may be increased through increasing washing time, volume of buffer, and number of washes. After the desired rounds of selection, the selected ssDNA or ssRNA pool may be PCR amplified and cloned into E. coli and sequenced. See Shangguan et al., Aptamers evolved from live cells as effective molecular probes for cancer study, PNAS 103(32:11838-11843 (2006); Lyu et al, Generating Cell Targeting Aptamers for Nanotherapeutics Using Cell-SELEX, Theranostics 6(9):1440-1452 (2016); see also Li et al., Inhibition of Cell Proliferation by an Anti-EGFR Aptamer, PLoS One 6(6):e20229 (2011). The specific approaches for designing aptamers and specific aptamers binding to cancer cells in these references are hereby incorporated by reference.

For example, an aptamer may comprise SEQ ID NO: 94 to 164. In some embodiments, an aptamer may comprise SEQ ID NO: 95. These aptamers are directed to EGFR and are provided only as representative of the aptamers that can bind to targets presented on cancer cells. Other aptamers against other targets on cancer cells are equally part of the description herein and incorporated by reference as described in Zhu et al., Progress in Aptamer Mediated Drug Delivery Vehicles for Cancer Targeting, Theranostics 4(9):931-944 (2014).

In some embodiments, aptamers for use herein bind to the target on the cancer cell with a K_{d} in the nanomolar to picomolar range (such as 1 picomolar to 500 nanomolar or 1 picomolar to 100 nanomolar).

Additional specific targeting moieties include those provided in Table 4C.

TABLE 4C
Selected examples of non-immunoglobulin and antigen-binding
fragments of antibodies that can serve as targeting molecules

| | Format | |
|---|---|---|
| Target Antigen | Scaffold | Reference |
| DKK1 | VHH | WO2010/130832 |
| c-Met | VHH | US2012/0244164 |
| TfR (CD71) | VNAR | US2017/0348416 |
| CD33 | Fynomer | WO2014/170063 |
| HLA-A*02:01 | TCR | IMCgp100 |
| gp100 | | |
| HLA-A*02:01NY- | TCR | US2018/0072788 |
| ESO | | |
| HER3 | Affibody | WO2014/053586A1 |
| HER2 | Affibody | US2010/0254899A1 |
| VEGF, HGF | DARPin | MP0250 |
| EGFR/HER2 | DARPin | US9499622B2 |
| EphA2 | Abdurin (CH2) | US2015/0353943 |

D. Immune Cell Engaging Domain

The immune cell engaging domain functions are capable of immune cell engaging activity when a first immune cell engaging domain binds to a second immune cell engaging domain. When the first and second immune cell engaging domains are paired together, when the inert binding partner is removed, they can bind to an immune cell. This binding can lead to activation of the immune cell.

In the absence of pairing of the first and second immune cell engaging domain, neither the first nor the second immune cell engaging domain alone can bind to an immune cell.

In some embodiments, the immune cell is a T cell, natural killer cell, macrophage, neutrophil, eosinophil, basophil, γδ T cell, NKT cell, or engineered immune cell. In some embodiments, the first and second immune cell engaging domains when paired together can activate an immune cell.

1. T-cell Engaging Domains

In some embodiments, the immune cell engaging domain is a T-cell engaging domain. The targeted T-cell engaging agent comprises a first T-cell engaging domain that is unable of engaging a T-cell alone. Instead, the first T-cell engaging domain is capable of activity when binding a second T-cell engaging domain, which is not part of the targeted T-cell engaging agent. Thus, the first and second T-cell engaging domains may be any two moieties that do not possess T-cell engaging activity alone, but do possess it when paired with each other. In other words, the first and second T-cell engaging domains are complementary halves of a functional active protein.

When the two T-cell engaging domains are associated together in the two-component system, they may bind to the CD3 antigen and/or T-cell receptor on the surface of the T-cell as these activate T cells. CD3 is present on all T cells and consists of subunits designated γ, δ, ε, ζ, and η. The cytoplasmic tail of CD3 is sufficient to transduce the signals necessary for T cell activation in the absence of the other components of the TCR receptor complex. Normally, activation of T cell cytotoxicity depends first on binding of the TCR with a major histocompatibility complex (MHC) protein, itself bound to a foreign antigen, located on a separate cell. In a normal situation, only when this initial TCR-MHC binding has taken place can the CD3 dependent signally cascade responsible for T cell clonal expansion and, ultimately, T cell cytotoxicity ensue. In some of the present embodiments, however, when the two-component system binds to CD3 and/or the TCR, activation of cytotoxic T cells in the absence of independent TCR-MHC can take place by virtue of the crosslinking of the CD3 and/or TCR molecules mimicking an immune synapse formation. This means that T cells may be cytotoxically activated in a clonally independent fashion, i.e. in a manner that is independent of the specific TCR clone carried by the T cell. This allows for activation of the entire T cell compartment rather than only specific T cells of a certain clonal identity.

In some embodiments, the first T-cell engaging domain is a VH domain and the second T-cell engaging domain is a VL domain. In other embodiments, the first T-cell engaging domain is a VL domain and the second T-cell engaging domain is a VH domain. In such embodiments, when paired together the first and second T-cell engaging domains may comprise an scFv (by this we mean equivalent to an scFv but for the fact that the VH and VL are not in a single-chain configuration).

If the first and second T-cell engaging domains are a pair of VH and VL domains, the VH and VL domains may be specific for an antigen expressed on the surface of a T cell, such as CD3 or TCR. If the antigen is CD3, one potential T-cell engaging domain may be derived from muromonab (muromonab-CD3 or OKT3), otelixizumab, teplizumab, visilizumab, foralumab, or SP34. One skilled in the art would be aware of a wide range of anti-CD3 antibodies, some of which are approved therapies or have been clinically tested in human patients (see Kuhn and Weiner Immunotherapy 8(8):889-906 (2016)). Table 5 presents selected publications on exemplary anti-CD3 antibodies.

TABLE 5
Selected References Showing Specificity of Exemplary Anti-CD3 Antibodies

| | |
|---|---|
| Muromonab/ | Herold K C et al. A single course of anti-CD3 monoclonal antibody |
| OKT3 | hOKT3gamma1(Ala-Ala) results in improvement in C-peptide responses |
| | and clinical parameters for at least 2 years after onset of type 1 diabetes. |
| | Diabetes. 54(6): 1763-9 (2005). |
| | Richards J et al. Phase I evaluation of humanized OKT3: toxicity and |
| | immunomodulatory effects of hOKT3gamma4. Cancer Res. 59(9): 2096-10 |
| | (1999). |
| | Kuhn C and Weiner H L. Therapeutic anti-CD3 monoclonal antibodies: |
| | from bench to bedside. Immunotherapy 8(8): 889-906 (2016). |
| Otelixizumab | Kuhn C et al. Human CD3 transgenic mice: preclinical testing of |
| | antibodies promoting immune tolerance. Sci Transl Med. 3(68): 68ra10 |
| | (2011). |
| | Kuhn C and Weiner H L. Therapeutic anti-CD3 monoclonal antibodies: |
| | from bench to bedside. Immunotherapy 8(8): 889-906 (2016). |
| | Dean Y et al. Combination therapies in the context of anti-CD3 |
| | antibodies for the treatment of autoimmune diseases. Swiss Med Wkly. |
| | 142: w13711 (2012). |
| | Daifotis A G et al. Anti-CD3 clinical trials in type 1 diabetes mellitus. |
| | Clin Immunol. 149(3): 268-78 (2013) (abstract). |
| | Chatenoud L and Waldmann H. CD3 monoclonal antibodies: a first step |
| | towards operational immune tolerance in the clinic. Rev Diabet Stud. |
| | 9(4): 372-81. (2012). |
| Teplizumab | Masharani U B and Becker J. Teplizumab therapy for type 1 diabetes. |
| | Expert Opin Biol Ther. 10(3): 459-65 (2010). |
| | Herold K C et al. Treatment of patients with new onset Type 1 diabetes |
| | with a single course of anti-CD3 mAb Teplizumab preserves insulin |
| | production for up to 5 years. Clin Immunol. 132(2): 166-73 (2009). |
| | Kuhn C and Weiner H L. Therapeutic anti-CD3 monoclonal antibodies: |
| | from bench to bedside. Immunotherapy 8(8): 889-906 (2016). |
| | Dean Y et al. Combination therapies in the context of anti-CD3 |
| | antibodies for the treatment of autoimmune diseases. Swiss Med Wkly. |
| | 142: w13711 (2012). |
| | Daifotis A G et al. Anti-CD3 clinical trials in type 1 diabetes mellitus. |
| | Clin Immunol. 149(3): 268-78 (2013) (abstract). |
| | Chatenoud L and Waldmann H. CD3 monoclonal antibodies: a first step |
| | towards operational immune tolerance in the clinic. Rev Diabet Stud. |
| | 9(4): 372-81 (2012). |
| Visilizumab | Kuhn C and Weiner H L. Therapeutic anti-CD3 monoclonal antibodies: |
| | from bench to bedside. Immunotherapy 8(8): 889-906 (2016). |
| | Shan L. ^{99m}Tc-Labeled succinimidyl-6-hydrazinonicotinate |
| | hydrochloride (SHNH)-conjugated visilizumab. Molecular Imaging and |
| | Contrast Agent Database (MICAD) [Internet]. Created: Dec. 7, 2009; |
| | Last Update: Jan. 12, 2010; Downloaded May 3, 2018. |
| | Dean Y et al. Combination therapies in the context of anti-CD3 |
| | antibodies for the treatment of autoimmune diseases. Swiss Med Wkly. |
| | 142: w13711 (2012). |
| Foralumab | Kuhn C and Weiner H L. Therapeutic anti-CD3 monoclonal antibodies: |
| | from bench to bedside. Immunotherapy 8(8): 889-906 (2016). |
| | Dean Y et al. Combination therapies in the context of anti-CD3 |
| | antibodies for the treatment of autoimmune diseases. Swiss Med Wkly. |
| | 142: w13711 (2012). |
| SP34 | Pessano S et al. The T3/T cell receptor complex: antigenic distinction |
| | between the two 20-kd T3 (T3-6 and T3-E) subunits. EMBO Journal |
| | 4(2): 337-344 (1985). |
| 20G6 | WO2016/116626 |

Antibodies with specificity to the TCR, including the αβ and γδ TCRs, are also well-known. Table 6 presents selected publications on exemplary anti-TCR antibodies.

TABLE 6
Selected References Showing Specificity of Exemplary Anti-TCR Antibodies

Verma-B. et al. TCR Mimic Monoclonal Antibody Targets a Specific Peptide/HLA Class I
Complex and Significantly Impedes Tumor Growth In Vivo Using Breast Cancer Models
J Immunol. 184: 2156-2165 (2010).
Conrad M L et al. TCR and CD3 antibody cross-reactivity in 44 species. Cytometry A.
71(11): 925-33 (2007).
Koenecke C et al. In vivo application of mAb directed against the gammadelta TCR does
not deplete but generates “invisible” gammadelta T cells. Eur J Immunol. 39(2): 372-9 (2009).
Exley M A et al. Selective activation, expansion, and monitoring of human iNKT cells with a
monoclonal antibody specific for the TCR alpha-chain CDR3 loop. Eur J Immunol. 38(6): 1756-
66 (2008).
Deetz C O et al. Gamma interferon secretion by human Vgamma2Vdelta2 T cells after
stimulation with antibody against the T-cell receptor plus the Toll-Like receptor 2 agonist
Pam3Cys. Infection and Immunity. 74(8): 4505-4511 (2006).
Tang X et al. Anti-TCR antibody treatment activates a novel population of nonintestinal
CD8 alpha alpha+ TCR alpha beta+ regulatory T cells and prevents experimental autoimmune
encephalomyelitis. J Immunol. 178(10): 6043-50 (2007).
Lavasani S et al. Monoclonal antibody against T-cell receptor alphabeta induces self-
tolerance in chronic experimental autoimmune encephalomyelitis. Scand J Immunol. 65(1): 39-
47 (2007).
Nasreen M et al. In vivo treatment of class II MHC-deficient mice with anti-TCR antibody
restores the generation of circulating CD4 T cells and optimal architecture of thymic medulla.
J Immunol. 171(7): 3394-400 (2003).

2. Natural Killer Cell Engaging Domains

In some embodiments, the immune cell engaging domain is a natural killer cell engaging domain. When the two natural killer cell engaging domains are associated together in the two-component system, they may bind to an antigen on the surface of the NK cell to engage these cells. In some embodiments, the antigen on the surface of the NK cell may be NKG2D, CD16, NKp30, NKp44, NKp46 or DNAM.

In some embodiments, having one half of the two-component system bind to a surface protein on the natural killer cell and having the other half of the system bind to cancer cells allows specific engagement of natural killer cells. Engagement of natural killer cells can lead to their activation and induce natural killer cell-mediated cytotoxicity and cytokine release.

When the two natural killer cell engaging domains are associated together in the ATTAC, the natural killer cell may specifically lyse the cancer cells bound by the cancer-specific ATTAC component. Killing of a cancer cell may be mediated by either the perforin/granzyme system or by FasL-Fas engagement. As well as this potential cytotoxic function, natural killer cells are also able to secrete pro-inflammatory cytokines including interferon gamma and tumor necrosis factor alpha which can activate macrophages and dendritic cells in the immediate vicinity to enhance the anti-cancer immune response.

In some embodiments, the first natural killer cell engaging domain is a VH domain and the second natural killer cell engaging domain is a VL domain. In other embodiments, the first natural killer cell engaging domain is a VL domain and the second natural killer cell engaging domain is a VH domain. In such embodiments, when paired together the first and second natural killer cell engaging domains may comprise an scFv (by this we mean equivalent to an scFv but for the fact that the VH and VL are not in a single-chain configuration).

If the first and second natural killer cell engaging domains are a pair of VH and VL domains, the VH and VL domains may be specific for an antigen expressed on the surface of a natural killer cell, such as NKG2D, CD16, NKp30, NKp44, NKp46 and DNAM.

Table 7 presents selected publications on some exemplary antibodies specific for an antigen expressed on the surface of a natural killer cell.

TABLE 7
Selected References Showing Specificity of Exemplary Antibodies
for Surface Antigens on Natural Killer Cells

| | |
|---|---|
| NKG2D | Vadstrup et al. Anti-NKG2D mAb: A New Treatment for Crohn's Disease? |
| | Int J Mol Sci. 18(9) (2017). |
| | Rong et al. Recognition and killing of cancer stem-like cell population in |
| | hepatocellular carcinoma cells by cytokine-induced killer cells via NKG2d- |
| | ligands recognition. Oncoimmunology. 5(3): e1086060 (2015). |
| | Shen et al. Possible association of decreased NKG2D expression levels and |
| | suppression of the activity of natural killer cells in patients with colorectal cancer. |
| | Int J Oncol. 40(4): 1285-90 (2012). |
| | Kim et al. Suppression of human anti-porcine natural killer cell xenogeneic |
| | responses by combinations of monoclonal antibodies specific to CD2 and |
| | NKG2D and extracellular signal-regulated kinase kinase inhibitor. |
| | Immunology. 130(4): 545-55 (2010). |
| | Steigerwald et al. Human IgG1 antibodies antagonizing activating receptor |
| | NKG2D on natural killer cells. MAbs. 1(2): 115-27 (2009). |
| | Paidipally et al. NKG2D-dependent IL-17 production by human T cells in |
| | response to an intracellular pathogen. J Immunol. 183(3): 1940-5 (2009). |
| | Kwong et al. Generation, affinity maturation, and characterization of a human |
| | anti-human NKG2D monoclonal antibody with dual antagonistic and agonistic |
| | activity. J Mol Biol. 384(5): 1143-56 (2008). |
| | Wrobel et al. Lysis of a broad range of epithelial tumour cells by human |
| | gamma delta T cells: involvement of NKG2D ligands and T-cell receptor- versus |
| | NKG2D-dependent recognition. Scand J Immunol. 66(2-3): 320-8 (2007). |
| | Andre et al. Comparative analysis of human NK cell activation induced by |
| | NKG2D and natural cytotoxicity receptors. Eur J Immunol. 34(4): 961-71 (2004). |
| | Regunathan et al. NKG2D receptor-mediated NK cell function is regulated by |
| | inhibitory Ly49 receptors. Blood. 105(1): 233-40 (2005). |
| CD16 | Lee et al. Expansion of cytotoxic natural killer cells using irradiated autologous |
| | peripheral blood mononuclear cells and anti-CD16 antibody. Sci Rep. 7(1): 11075 |
| | (2017). |
| | Parsons et al. Anti-HIV antibody-dependent activation of NK cells impairs |
| | NKp46 expression. J Immunol. 192(1): 308-15 (2014). |
| | Vallera et al. Heterodimeric bispecific single-chain variable-fragment |
| | antibodies against EpCAM and CD16 induce effective antibody-dependent |
| | cellular cytotoxicity against human carcinoma cells. Cancer Biother Radiopharm. |
| | 28(4): 274-82 (2013). |
| | Asano et al. Construction and humanization of a functional bispecific EGFR × |
| | CD16 diabody using a refolding system. FEBS J. 279(2): 223-33 (2012). |
| | Jewett et al. Strategies to rescue mesenchymal stem cells (MSCs) and dental |
| | pulp stem cells (DPSCs) from NK cell mediated cytotoxicity. PLoS One. |
| | 5(3): e9874 (2010). |
| | Congy-Jolivet et al. Fc gamma RIIIa expression is not increased on natural |
| | killer cells expressing the Fc gamma RIIIa-158V allotype. Cancer Res. |
| | 68(4): 976-80 (2008). |
| | Jewett et al. Coengagement of CD16 and CD94 receptors mediates secretion of |
| | chemokines and induces apoptotic death of naive natural killer cells. |
| | Clin Cancer Res. 12(7 Pt 1): 1994-2003 (2006). |
| | Yamaguchi et al. HER2-specific cytotoxic activity of lymphokine-activated |
| | killer cells in the presence of trastuzumab. Anticancer Res. 25(2A): 827-32 |
| | (2005). |
| | Shahied et al. Bispecific minibodies targeting HER2/neu and CD16 exhibit |
| | improved tumor lysis when placed in a divalent tumor antigen binding format. |
| | J Biol Chem. 279(52): 53907-14 (2004). |
| | Dall'Ozzo et al. Rituximab-dependent cytotoxicity by natural killer cells: |
| | influence of FCGR3A polymorphism on the concentration-effect relationship. |
| | Cancer Res. 64(13): 4664-9 (2004). |
| NKp30 | Hervier et al. Involvement of NK Cells and NKp30 Pathway in Antisynthetase |
| | Syndrome. J Immunol. 197(5): 1621-30 (2016). |
| | Zou et al. NKP30-B7-H6 Interaction Aggravates Hepatocyte Damage through |
| | Up-Regulation of Interleukin-32 Expression in Hepatitis B Virus-Related Acute- |
| | On-Chronic Liver Failure. PLoS One. 10(8): e0134568 (2015). |
| | Ferrari de Andrade et al. Natural killer cells are essential for the ability of |
| | BRAF inhibitors to control BRAFV600E-mutant metastatic melanoma. |
| | Cancer Res. 74(24): 7298-308 (2014). |
| | Warren et al. Evidence that the cellular ligand for the human NK cell activation |
| | receptor NKp30 is not a heparan sulfate glycosaminoglycan. J Immunol. |
| | 175(1): 207-12 (2005). |
| | Holder et al. Hepatitis C virus-infected cells downregulate NKp30 and inhibit |
| | ex vivo NK cell functions. J Immunol. 191(6): 3308-18 (2013). |
| | Laufer et al. CD4+ T cells and natural killer cells: Biomarkers for hepatic |
| | fibrosis in human immunodeficiency virus/hepatitis C virus-coinfected patients. |
| | World J Hepatol. 9(25): 1073-1080 (2017). |
| | Chretien et al. NKp30 expression is a prognostic immune biomarker for |
| | stratification of patients with intermediate-risk acute myeloid leukemia. |
| | Oncotarget. 8(30): 49548-49563 (2017). |
| | Spaggiari et al. Mesenchymal stem cells inhibit natural killer-cell proliferation, |
| | cytotoxicity, and cytokine production: role of indoleamine 2,3-dioxygenase and |
| | prostaglandin E2. Blood. 111: 1327-1333 (2008). |
| | Fiegler et al. Downregulation of the activating NKp30 ligand B7-H6 by HDAC |
| | inhibitors impairs tumor cell recognition by NK cells. Blood. 122(5): 684-693 |
| | (2013). |
| | Salimi et al. Group 2 Innate Lymphoid Cells Express Functional NKp30 |
| | Receptor Inducing Type 2 Cytokine Production. J Immunol. 196: 45-54 (2016). |
| NKp44 | Esin et al. Interaction of Mycobacterium tuberculosis cell wall components |
| | with the human natural killer cell receptors NKp44 and Toll-like receptor 2. |
| | Scand J Immunol. 77(6): 460-9 (2013). |
| | Hershkovitz et al. NKp44 receptor mediates interaction of the envelope |
| | glycoproteins from the West Nile and dengue viruses with NK cells. J Immunol. |
| | 2009 Aug. 15; 183(4): 2610-21. |
| | Sivori et al. 2B4 functions as a co-receptor in human NK cell activation. |
| | Eur J Immunol. 30(3): 787-93 (2000). |
| | Vitale et al. NKp44, a Novel Triggering Surface Molecule Specifically |
| | Expressed by Activated Natural Killer Cells, Is Involved in Non-Major |
| | Histocompatibility Complex-restricted Tumor Cell Lysis. J Exp. Med. |
| | 187(12): 2065-2072 (1998). |
| | Campbell et al. NKp44 Triggers NK Cell Activation through DAP12 |
| | Association That Is Not Influenced by a Putative Cytoplasmic Inhibitory |
| | Sequence. J Immunol. 172: 899-906 (2004). |
| | Fuchs et al. Paradoxic inhibition of human natural interferon-producing cells |
| | by the activating receptor NKp44. Blood. 106: 2076-2082 (2005). |
| | Vacca et al. Regulatory role of NKp44, NKp46, DNAM-1 and NKG2D |
| | receptors in the interaction between NK cells and trophoblast cells. Evidence for |
| | divergent functional profiles of decidual versus peripheral NK cells. |
| | International Immunology 20(11): 1395-1405 (2008). |
| | Cantoni et al. NKp44, A Triggering Receptor Involved in Tumor Cell Lysis by |
| | Activated Human Natural Killer Cells, Is a Novel Member of the |
| | Immunoglobulin Superfamily. J Exp Med. 189(5): 787-795 (1999). |
| | Vieillard et al. NK cytotoxicity against CD4+ T cells during HIV-1 infection: |
| | A gp41 peptide induces the expression of an NKp44 ligand. Proc Natl Acad Sci U S A. |
| | 102(31): 10981-10986. |
| | Glatzer et al. RORγt + Innate Lymphoid Cells Acquire a Proinflammatory |
| | Program upon Engagement of the Activating Receptor NKp44. Immunity. |
| | 38: 1223-1235 (2013). |
| NKp46 | Shemer-Avni et al. Expression of NKp46 Splice Variants in Nasal Lavage |
| | Following Respiratory Viral Infection: Domain 1-Negative Isoforms Predominate |
| | and Manifest Higher Activity. Front Immunol. 8: 161 (2017). |
| | Crome et al. A distinct innate lymphoid cell population regulates tumor- |
| | associated T cell Nat Med. 23(3): 368-375 (2017). |
| | Li et al. Natural Killer p46 Controls Hepatitis B Virus Replication and |
| | Modulates Liver Inflammation. PLoS One. 10(8): e0135874 (2015). |
| | Dou et el. Influenza vaccine induces intracellular immune memory of human |
| | NK cells. PLoS One. 10(3): e0121258 (2015). |
| | Vego et al. Monomethyl fumarate augments NK cell lysis of tumor cells |
| | through degranulation and the upregulation of NKp46 and CD107a. |
| | Cell Mol Immunol. 13(1): 57-64 (2016). |
| | Vankayalapati et al. Role of NK cell-activating receptors and their ligands in |
| | the lysis of mononuclear phagocytes infected with an intracellular bacterium. |
| | J Immunol. 175(7): 4611-7 (2005). |
| | Laufer et al. CD4+ T cells and natural killer cells: Biomarkers for hepatic |
| | fibrosis in human immunodeficiency virus/hepatitis C virus-coinfected patients. |
| | World J Hepatol. 9(25): 1073-1080 (2017). |
| | Yoshioka et al. Frequency and role of NKp46 and NKG2A in hepatitis B virus |
| | infection. PLoS One. 12(3): e0174103 (2017). |
| | Vacca et al. Regulatory role of NKp44, NKp46, DNAM-1 and NKG2D |
| | receptors in the interaction between NK cells and trophoblast cells. Evidence for |
| | divergent functional profiles of decidual versus peripheral NK cells. |
| | International Immunology 20(11): 1395-1405 (2008). |
| DNAM | Okumura G, et al. Development and Characterization of Novel Monoclonal |
| (CD226) | Antibodies Against Human DNAM-1. |
| | Monoclon Antib Immunodiagn Immunother. |
| | 36(3): 135-139 (2017). |
| | Stein N et al. The paired receptors TIGIT and DNAM-1 as targets for |
| | therapeutic antibodies. Hum Antibodies. 25(3-4): 111-119 (2017). |
| | Elhai M et al. Targeting CD226/DNAX accessory molecule-1 (DNAM-1) in |
| | collagen-induced arthritis mouse models. J Inflamm (Lond). 12: 9 (2015). |
| | Laufer et al. CD4+ T cells and natural killer cells: Biomarkers for hepatic |
| | fibrosis in human immunodeficiency virus/hepatitis C virus-coinfected patients. |
| | World J Hepatol. 9(25): 1073-1080 (2017). |
| | Shibuya et al. Physical and Functional Association of LFA-1 with DNAM-1 |
| | Adhesion Molecule. Immunity. 11: 615-623 (1999). |
| | Li et al. CD155 loss enhances tumor suppression via combined host and tumor- |
| | intrinsic mechanisms. J Clin Invest. pii: 98769 (2018). |
| | Chen et al. Targeting chemotherapy-resistant leukemia by combining DNT |
| | cellular therapy with conventional chemotherapy. J Exp Clin Cancer Res. |
| | 37(1): 88 (2018). |
| | Rodrigues et al. Low-Density Lipoprotein Uptake Inhibits the Activation and |
| | Antitumor Functions of Human Vγ9Vδ2 T Cells. Cancer Immunol Res. 6(4): 448- |
| | 457 (2018). |
| | Rocca et al. Phenotypic and Functional Dysregulated Blood NK Cells in |
| | Colorectal Cancer Patients Can Be Activated by Cetuximab Plus IL-2 or IL-15. |
| | Front Immunol. 7: 413 (2016). |
| | Shibuya et al DNAM-1, a novel adhesion molecule involved in the cytolytic |
| | function of T lymphocytes. Immunity. 4(6): 573-81(1996). |

3. Macrophage Engaging Domains

In some embodiments, the immune cell engaging domain is a macrophage engaging domain. As used herein, a “macrophage” may refer to any cell of the mononuclear phagocytic system, such as grouped lineage-committed bone marrow precursors, circulating monocytes, resident macrophages, and dendritic cells (DC). Examples of resident macrophages can include Kupffer cells and microglia.

When the two macrophage engaging domains are associated together in the two-component system, they may bind to an antigen on the surface of the macrophage to engage these cells. In some embodiments, the antigen on the surface of the macrophage may be CD89 (Fc alpha receptor 1), CD64 (Fc gamma receptor 1), CD32 (Fc gamma receptor 2A) or CD16a (Fc gamma receptor 3A).

In some embodiments, having one half of the two-component system bind to a surface protein on the macrophage and having the other half of the system bind to cancer cells allows specific engagement of macrophages. Engagement of macrophages can lead the macrophage to phagocytose the cancer cell.

In some embodiments, inducing macrophage phagocytosis via binding to an antigen on the surface of the macrophages is independent of Fc receptor binding, which has been shown previously to be a method of tumor cell killing by macrophages. Normally, cancer cells are bound by whole antibodies and the Fc portion of the antibody binds to the Fc receptor and induces phagocytosis.

In some embodiments, engagement of toll-like receptors on the macrophage surface (see patent application US20150125397A1) leads to engagement of macrophages.

When the two macrophage engaging domains are associated together in the ATTAC, they may induce the macrophage to phagocytose the cancer cell bound by the cancer-specific ATTAC component.

In some embodiments, the first macrophage engaging domain is a VH domain and the second macrophage engaging domain is a VL domain. In other embodiments, the first macrophage engaging domain is a VL domain and the second macrophage engaging domain is a VH domain. In such embodiments, when paired together the first and second macrophage engaging domains may comprise an scFv (by this we mean equivalent to an scFv but for the fact that the VH and VL are not in a single-chain configuration).

If the first and second macrophage engaging domains are a pair of VH and VL domains, the VH and VL domains may be specific for an antigen expressed on the surface of a macrophage, such as CD89 (Fc alpha receptor 1), CD64 (Fc gamma receptor 1), CD32 (Fc gamma receptor 2A) and CD16a (Fc gamma receptor 3A), or toll-like receptors.

Table 8 presents selected publications on some exemplary antibodies specific for an antigen expressed on the surface of a macrophage.

TABLE 8
Selected References Showing Specificity of Exemplary
Antibodies for Surface Antigens on Macrophages

| | |
|---|---|
| CD89 (Fc | Xu et al. Critical Role of Kupffer Cell CD89 Expression in |
| alpha | Experimental IgA Nephropathy. PLoS One. 11(7): e0159426 (2016). |
| receptor | Deo et al. Bispecific molecules directed to the Fc receptor for IgA (Fc |
| 1) | alpha RI, CD89) and tumor antigens efficiently promote cell-mediated |
| | cytotoxicity of tumor targets in whole blood. J Immunol. 160(4): 1677-86 |
| | (1998). |
| | Hamre et al. Expression and modulation of the human |
| | immunoglobulin A Fc receptor (CD89) and the FcR gamma chain on |
| | myeloid cells in blood and tissue. Scand J Immunol. 57(6): 506-16 |
| | (2003). |
| | Mladenov et al. The Fc-alpha receptor is a new target antigen for |
| | immunotherapy of myeloid leukemia. Int J Cancer. 137(11): 2729-38 |
| | (2015). |
| | United States Patent Application US20110104145A1 Method for the |
| | treatment or prophylaxis of chronic inflammatory diseases. |
| | Smith et al. Intestinal macrophages lack CD14 and CD89 and |
| | consequently are down-regulated for LPS- and IgA-mediated activities. |
| | J Immunol. 167(5): 2651-6 (2001). |
| | Van Zandbergen et al. Crosslinking of the human Fc receptor for IgA |
| | (FcalphaRI/CD89) triggers FcR gamma-chain-dependent shedding of |
| | soluble CD89. J Immunol. 163(11): 5806-12 (1999). |
| | Cheeseman et al. Expression Profile of Human Fc Receptors in |
| | Mucosal Tissue: Implications for Antibody-Dependent Cellular Effector |
| | Functions Targeting HIV-1 Transmission. PLoS One. 11(5): e0154656 |
| | (2016). |
| | Geissman et al. A subset of human dendritic cells expresses IgA Fc |
| | receptor (CD89), which mediates internalization and activation upon |
| | cross-linking by IgA complexes. J Immunol. 166(1): 346-52 (2001). |
| | Reterink et al. Transforming growth factor-beta 1 (TGF-beta 1) down- |
| | regulates IgA Fc-receptor (CD89) expression on human monocytes. |
| | Clin Exp Immunol. 103(1): 161-6 (1996). |
| CD64 (Fc | Histodorov et al. Recombinant H22(scFv) blocks CD64 and prevents |
| gamma | the capture of anti-TNF monoclonal antibody. A potential strategy to |
| receptor | enhance anti-TNF therapy. MAbs. 6(5): 1283-9 (2014). |
| 1) | Cheeseman et al. Expression Profile of Human Fc Receptors in |
| | Mucosal Tissue: Implications for Antibody-Dependent Cellular Effector |
| | Functions Targeting HIV-1 Transmission. PLoS One. 11(5): e0154656 |
| | (2016). |
| | Moura et al. Co-association of methotrexate and SPIONs into anti- |
| | CD64 antibody-conjugated PLGA nanoparticles for theranostic |
| | application. Int J Nanomedicine. 9: 4911-22 (2014). |
| | Petricevic et al. Trastuzumab mediates antibody-dependent cell- |
| | mediated cytotoxicity and phagocytosis to the same extent in both |
| | adjuvant and metastatic HER2/neu breast cancer patients. J Transl Med. |
| | 11: 307 (2013). |
| | Miura et al. Paclitaxel enhances antibody-dependent cell-mediated |
| | cytotoxicity of trastuzumab by rapid recruitment of natural killer cells in |
| | HER2-positive breast cancer. J Nippon Med Sch. 81(4): 211-20 (2014). |
| | Schiffer et al. Targeted ex vivo reduction of CD64-positive monocytes |
| | in chronic myelomonocytic leukemia and acute myelomonocytic |
| | leukemia using human granzyme B-based cytolytic fusion proteins. |
| | Int J Cancer. 135(6): 1497-508 (2014). |
| | Matt et al. Elevated Membrane and Soluble CD64: A Novel Marker |
| | Reflecting Altered FcγR Function and Disease in Early Rheumatoid |
| | Arthritis That Can Be Regulated by Anti-Rheumatic Treatment. |
| | PLoS One. 10(9): e0137474 (2015). |
| | Haegel et al. A unique anti-CD115 monoclonal antibody which |
| | inhibits osteolysis and skews human monocyte differentiation from M2- |
| | polarized macrophages toward dendritic cells. MAbs. 5(5): 736-47 (2013). |
| | Mladenov et al. CD64-directed microtubule associated protein tau kills |
| | leukemic blasts ex vivo. Oncotarget. 7(41): 67166-67174 (2016). |
| | Wong et al. Monochromatic gating method by flow cytometry for high |
| | purity monocyte analysis. Cytometry B Clin Cytom. 84(2): 119-24 (2013). |
| CD32 (Fc | Cheeseman et al. Expression Profile of Human Fc Receptors in |
| gamma | Mucosal Tissue: Implications for Antibody-Dependent Cellular Effector |
| receptor | Functions Targeting HIV-1 Transmission. PLoS One. 11(5): e0154656 |
| 2A) | (2016). |
| | Bhatnagar et al. FcγRIII (CD16)-mediated ADCC by NK cells is |
| | regulated by monocytes and FcγRII (CD32). Eur J Immunol. |
| | 44(11): 3368-79 (2014). |
| | Veri et al. Monoclonal antibodies capable of discriminating the human |
| | inhibitory Fcgamma-receptor IIB (CD32B) from the activating |
| | Fcgamma-receptor IIA (CD32A): biochemical, biological and functional |
| | characterization. Immunology. 121(3): 392-404 (2007). |
| | Vivers et al. Divalent cation-dependent and -independent |
| | augmentation of macrophage phagocytosis of apoptotic neutrophils by |
| | CD44 antibody. Clin Exp Immunol. 138(3): 447-52 (2004). |
| | Athanasou et al. Immunophenotypic differences between osteoclasts |
| | and macrophage polykaryons: immunohistological distinction and |
| | implications for osteoclast ontogeny and function. J Clin Pathol. |
| | 43(12): 997-1003 (1990). |
| | Leidi et al. M2 macrophages phagocytose rituximab-opsonized |
| | leukemic targets more efficiently than m1 cells in vitro. J Immunol. |
| | 182(7): 4415-22 (2009). |
| | Shanaka et al. Differential Enhancement of Dengue Virus Immune |
| | Complex Infectivity Mediated by Signaling-Competent and Signaling- |
| | Incompetent Human FcγRIA (CD64) or FcγRIIA (CD32). J Virol. |
| | 80(20): 10128-10138 (2006). |
| | Lee et al. Isolation and immunocytochemical characterization of |
| | human bone marrow stromal macrophages in hemopoietic clusters. |
| | J Exp Med. 168(3): 1193-8 (1988). |
| | Dialynas et al. Phenotypic and functional characterization of a new |
| | human macrophage cell line K1m demonstrating immunophagocytic |
| | activity and signalling through HLA class II. Immunology. 90(4): 470-6 |
| | (1997). |
| | Athanasou et al. Immunocytochemical analysis of human synovial |
| | lining cells: phenotypic relation to other marrow derived cells. |
| | Ann Rheum Dis. 50(5): 311-315 (1991). |
| CD 16a | Zhou et al. CD14(hi)CD16+ monocytes phagocytose antibody- |
| (Fc | opsonised Plasmodium falciparum infected erythrocytes more efficiently |
| gamma | than other monocyte subsets, and require CD16 and complement to do |
| receptor | so. BMC Med. 13: 154(2015). |
| 3A) | Cheeseman et al. Expression Profile of Human Fc Receptors in |
| | Mucosal Tissue: Implications for Antibody-Dependent Cellular Effector |
| | Functions Targeting HIV-1 Transmission. PLoS One. 11(5): e0154656 |
| | (2016). |
| | Dialynas et al. Phenotypic and functional characterization of a new |
| | human macrophage cell line K1m demonstrating immunophagocytic |
| | activity and signalling through HLA class II. Immunology. 90(4): 470-6 |
| | (1997). |
| | Nazareth et al. Infliximab therapy increases the frequency of |
| | circulating CD16(+) monocytes and modifies macrophage cytokine |
| | response to bacterial infection. Clin Exp Immunol. 2014 September; 177(3): 703- |
| | 11. |
| | Pander et al. Activation of tumor-promoting type 2 macrophages by |
| | EGFR-targeting antibody cetuximab. Clin Cancer Res. 17(17): 5668-73 |
| | (2011). |
| | Boyle. Human macrophages kill human mesangial cells by Fas-L- |
| | induced apoptosis when triggered by antibody via CD16. |
| | Clin Exp Immunol. 137(3): 529-37 (2004). |
| | Korkosz et al. Monoclonal antibodies against macrophage colony- |
| | stimulating factor diminish the number of circulating intermediate and |
| | nonclassical (CD14(++)CD16(+)/CD14(+)CD16(++)) monocytes in |
| | rheumatoid arthritis patient. Blood. 119(22): 5329-30 (2012). |
| | Wang et al. Interleukin-10 induces macrophage apoptosis and |
| | expression of CD16 (FcgammaRIII) whose engagement blocks the cell |
| | death programme and facilitates differentiation. Immunology. |
| | 102(3): 331-7 (2001). |
| | Kramer et al. 17 beta-estradiol regulates cytokine release through |
| | modulation of CD16 expression in monocytes and monocyte-derived |
| | macrophages. Arthritis Rheum. 50(6): 1967-75 (2004). |
| | Tricas et al. Flow cytometry counting of bronchoalveolar lavage |
| | leukocytes with a new profile of monoclonal antibodies combination. |
| | Cytometry B Clin Cytom. 82(2): 61-6 (2012). |

4. Neutrophil Engaging Domains

In some embodiments, the immune cell engaging domain is a neutrophil engaging domain. When the two neutrophil engaging domains are associated together in the two-component system, they may bind to an antigen on the surface of the neutrophil to engage these cells. In some embodiments, the antigen on the surface of the neutrophil may be CD89 (FcαR1), FcγRI (CD64), FcγRIIA (CD32), FcγRIIIA (CD16a), CD11b (CR3, αMβ2), TLR2, TLR4, CLEC7A (Dectin1), formyl peptide receptor 1 (FPR1), formyl peptide receptor 2 (FPR2), or formyl peptide receptor 3 (FPR3).

In some embodiments, having one half of the two-component system bind to a surface protein on the neutrophil and having the other half of the system bind to cancer cells allows specific engagement of neutrophils. Engagement of neutrophils can lead to phagocytosis and cell uptake.

When the two neutrophil engaging domains are associated together in the ATTAC, the neutrophil may engulf the target cells.

In some embodiments, the first neutrophil engaging domain is a VH domain and the second neutrophil engaging domain is a VL domain. In other embodiments, the first neutrophil engaging domain is a VL domain and the second neutrophil engaging domain is a VH domain. In such embodiments, when paired together the first and second neutrophil engaging domains may comprise an scFv (by this we mean equivalent to an scFv but for the fact that the VH and VL are not in a single-chain configuration).

If the first and second neutrophil engaging domains are a pair of VH and VL domains, the VH and VL domains may be specific for an antigen expressed on the surface of a neutrophil, such as CD89 (FcαR1), FcγRI (CD64), FcγRIIA (CD32), FcγRIIIA (CD16a), CD11b (CR3, αMβ2), TLR2, TLR4, CLEC7A (Dectin1), FPR1, FPR2, or FPR3.

Table 9 presents selected publications on some exemplary antibodies specific for an antigen expressed on the surface of a neutrophil.

TABLE 9
Selected References Showing Specificity of Exemplary
Antibodies for Surface Antigens on Neutrophils

| CD89 (FcαR1) | Li B et al. CD89-mediated recruitment of macrophages via a |
|---|---|
| | bispecific antibody enhances anti-tumor efficacy. Oncoimmunology. |
| | 7(1) (2017) |
| | Valerius T et al. FcalphaRI (CD89) as a novel trigger molecule for |
| | bispecific antibody therapy. Blood 90(11): 4485-92 (1997) |
| FcγRI (CD64) | Honeychurch et al. Therapeutic efficacy of FcgammaRI/CD64- |
| | directed bispecific antibodies in B-cell lymphoma. Blood |
| | 96(10): 3544-52 (2000) |
| | James et al. A phase II study of the bispecific antibody MDX-H210 |
| | (anti-HER2 × CD64) with GM-CSF in HER2+ advanced prostate |
| | cancer. British Journal of Cancer 85(2): 152-156 (2001) |
| | Futosi K et al Neutrophil cell surface receptors and their |
| | intracellular signal transduction pathways. Int Immunopharmacol. |
| | 17(3): 638-50 (2013) |
| | Kasturirangan et al. Targeted Fcγ Receptor (FcγR)-mediated |
| | Clearance by a Biparatopic Bispecific Antibody. |
| | Journal of Biological Chemistry 292(10): |
| | 4361-4370 (2017) |
| FcγRIIA | Futosi K et al Neutrophil cell surface receptors and their |
| (CD32) | intracellular signal transduction pathways. Int Immunopharmacol. |
| | 17(3): 638-50 (2013) |
| | Nimmerjahn F and Ravetch J V. Antibodies, Fc receptors and |
| | cancer. Curr Opin Immunol. 19(2): 239-45 (2007) |
| | Ravetch J V: Fc receptors. In Fundamental Immunology, edn5. |
| | Edited by Paul W E. Lippincott-Raven; 685-700 (2003) |
| | Nimmerjahn F, Ravetch J V: Fcγ receptors: old friends and new |
| | family members. Immunity 24: 19-28 (2006) |
| FcγRIIIA | Futosi K et al Neutrophil cell surface receptors and their |
| (CD16a) | intracellular signal transduction pathways. Int Immunopharmacol. |
| | 17(3): 638-50 (2013) |
| | Nimmerjahn F and Ravetch J V. Antibodies, Fc receptors and |
| | cancer. Curr Opin Immunol. 19(2): 239-45 (2007) |
| | Ravetch J V: Fc receptors. In Fundamental Immunology, edn5. |
| | Edited by Paul W E. Lippincott-Raven; 685-700 (2003) |
| | Nimmerjahn F, Ravetch J V Fcγ receptors: old friends and new |
| | family members. Immunity 24: 19-28 (2006) |
| | Renner et al. Targeting properties of an anti-CD16/anti-CD30 |
| | bispecific antibody in an in vivo system. |
| | Cancer Immunol Immunother. 50(2): |
| | 102-8 (2001) |
| CD11b(CR3, | Gazendam R P et al. How neutrophils kill fungi. Immunol Rev. |
| (α_{M}β₂) | 273(1): 299-311 (2016) |
| | Urbaczek A C et al. Influence of FcγRIIIb polymorphism on its |
| | ability to cooperate with FcγRIIa and CR3 in mediating the oxidative |
| | burst of human neutrophils. Hum Immunol. 75(8): 785-90 (2014) |
| | Futosi K et al Neutrophil cell surface receptors and their |
| | intracellular signal transduction pathways. Int Immunopharmacol. |
| | 17(3): 638-50 (2013) |
| | van Spriel A B et al. Mac-1 (CD11b/CD18) is essential for Fc |
| | receptor-mediated neutrophil cytotoxicity and immunologic synapse |
| | formation. Blood. 97(8): 2478-86 (2001) |
| TLR2 | Kawasaki T and Kawai T. Toll-Like Receptor Signaling Pathways. |
| | Front Immunol. 5: 461 (2014) |
| | Beutler B A. TLRs and innate immunity. Blood. 113(7): 1399-407 |
| | (2009) |
| | Beutler B et al. Genetic analysis of host resistance: Toll-like |
| | receptor signaling and immunity at large. Annu Rev Immunol. 24: 353- |
| | 89 (2006) |
| TLR4 | Kawasaki T and Kawai T. Toll-Like Receptor Signaling Pathways. |
| | Front Immunol. 5: 461 (2014) |
| | Beutler B A. TLRs and innate immunity. Blood. 113(7): 1399-407 |
| | (2009) |
| | Beutler B et al. Genetic analysis of host resistance: Toll-like |
| | receptor signaling and immunity at large. Annu Rev Immunol. 24: 353- |
| | 89 (2006) |
| CLEC7A | Brown G D. Dectin-1: a signalling non-TLR pattern-recognition |
| (Dectin1) | receptor. Nat Rev Immunol. 6(1): 33-43 (2006) |
| | Pyz E et al. C-type lectin-like receptors on myeloid cells. Ann Med. |
| | 38(4): 242-51 (2006) |
| FPR1, FPR2, | Dahlgren C et al. Basic characteristics of the neutrophil receptors |
| FPR3 | that recognize formylated peptides, a danger-associated molecular |
| | pattern generated by bacteria and mitochondria. Biochem Pharmacol. |
| | 114: 22-39. doi: 10.1016/j.bcp.2016.04.014 (2016) |
| | Lee HY et al. Formyl Peptide Receptors in Cellular Differentiation |
| | and Inflammatory Diseases. J Cell Biochem. 118(6): 1300-1307 |
| | (2017) |

5. Eosinophil Engaging Domains

In some embodiments, the immune cell engaging domain is an eosinophil engaging domain. When the two eosinophil engaging domains are associated together in the two-component system, they may bind to an antigen on the surface of the eosinophil to engage these cells. In some embodiments, the antigen on the surface of the eosinophil may be CD89 (Fc alpha receptor 1), FcεRI, FcγRI (CD64), FcγRIIA (CD32), FcγRIIIB (CD16b), or TLR4.

In some embodiments, having one half of the two-component system bind to a surface protein on the eosinophil and having the other half of the system bind to cancer cells allows specific engagement of eosinophils. Engagement of eosinophils can lead to degranulation and release of preformed cationic proteins, such as EPO, major basic protein 1 (MBP1), and eosinophil-associated ribonucleases (EARs), known as ECP and eosinophil-derived neurotoxin.

When the two neutrophil engaging domains are associated together in the ATTAC, the neutrophil may phagocytose the target cell or secrete neutrophil extracellular traps (NETs); finally, they may activate their respiratory burst cascade to kill phagocytosed cells.

In some embodiments, the first eosinophil engaging domain is a VH domain and the second eosinophil engaging domain is a VL domain. In other embodiments, the first eosinophil engaging domain is a VL domain and the second eosinophil engaging domain is a VH domain. In such embodiments, when paired together the first and second eosinophil engaging domains may comprise an scFv (by this we mean equivalent to an scFv but for the fact that the VH and VL are not in a single-chain configuration).

If the first and second eosinophil engaging domains are a pair of VH and VL domains, the VH and VL domains may be specific for an antigen expressed on the surface of an eosinophil, such as CD89 (Fc alpha receptor 1), FcεRI, FcγRI (CD64), FcγRIIA (CD32), FcγRIIIB (CD16b), or TLR4.

Table 10 presents selected publications on some exemplary antibodies specific for an antigen expressed on the surface of an eosinophil.

TABLE 10
Selected References Showing Specificity of Exemplary
Antibodies for Surface Antigens on Eosinophils

| | |
|---|---|
| CD89 (Fc | Xu et al. Critical Role of Kupffer Cell CD89 Expression in |
| alpha | Experimental IgA Nephropathy. PLoS One. 11(7): e0159426 (2016) |
| receptor 1) | Monteiro R C et al. IgA Fc receptors. Annu Rev Immunol. 21: 177-204. |
| | (2003) |
| | Morton H C et al. CD89: the human myeloid IgA Fc receptor. |
| | Arch Immunol Ther Exp (Warsz). 49(3): 217-29 (2001) |
| FcεRI | Stone K D et al. IgE, mast cells, basophils, and eosinophils. |
| | J Allergy Clin Immunol. 125(2 Suppl 2): S73-80 (2010) |
| | Conner E R and Saini S S. The immunoglobulin E receptor: expression |
| | and regulation. Curr Allergy Asthma Rep. 5(3): 191-6 (2005) |
| FcγRI | Nimmerjahn F and Ravetch J V. Antibodies, Fc receptors and cancer. |
| (CD64) | Curr Opin Immunol. 19(2): 239-45 (2007) |
| | Ravetch J V: Fc receptors. In Fundamental Immunology, edn5. Edited |
| | by Paul W E. Lippincott-Raven; 685-700 (2003) |
| | Nimmerjahn F, Ravetch J V: Fcγ receptors: old friends and new family |
| | members. Immunity 24: 19-28 (2006) |
| FcγRIIA | Nimmerjahn F and Ravetch J V. Antibodies, Fc receptors and cancer. |
| (CD32) | Curr Opin Immunol. 19(2): 239-45 (2007) |
| | Ravetch J V: Fc receptors. In Fundamental Immunology, edn5. Edited |
| | by Paul W E. Lippincott-Raven; 685-700 (2003) |
| | Nimmerjahn F, Ravetch J V: Fcγ receptors: old friends and new family |
| | members. Immunity 24: 19-28 (2006) |
| FcγRIIIB | Nimmerjahn F and Ravetch J V. Antibodies, Fc receptors and cancer. |
| (CD 16b) | Curr Opin Immunol. 19(2): 239-45 (2007) |
| | Ravetch J V: Fc receptors. In Fundamental Immunology, edn5. Edited |
| | by Paul W E. Lippincott-Raven; 685-700 (2003) |
| | Nimmerjahn F, Ravetch J V: Fcγ receptors: old friends and new family |
| | members. Immunity 24: 19-28 (2006) |
| TLR4 | Beutler B A. TLRs and innate immunity. Blood 113(7): 1399-407 |
| | (2009) |
| | Beutler B et al. Genetic analysis of host resistance: Toll-like receptor |
| | signaling and immunity at large. Annu Rev Immunol. 24: 353-89 (2006) |

6. Basophil Engaging Domains

In some embodiments, the immune cell engaging domain is a basophil engaging domain. When the two basophil engaging domains are associated together in the two-component system, they may bind to an antigen on the surface of the basophil to engage these cells. In some embodiments, the antigen on the surface of the basophil may be CD89 (Fc alpha receptor 1) or FcεRI.

In some embodiments, having one half of the two-component system bind to a surface protein on the basophil and having the other half of the system bind to cancer cells allows specific engagement of basophils. Engagement of basophils can lead to the release of basophil granule components such as histamine, proteoglycans, and proteolytic enzymes. They also secrete leukotrienes (LTD-4) and cytokines.

When the two basophil engaging domains are associated together in the ATTAC, the basophil may degranulate.

In some embodiments, the first basophil engaging domain is a VH domain and the second basophil engaging domain is a VL domain. In other embodiments, the first basophil engaging domain is a VL domain and the second basophil engaging domain is a VH domain. In such embodiments, when paired together the first and second basophil engaging domains may comprise an scFv (by this we mean equivalent to an scFv but for the fact that the VH and VL are not in a single-chain configuration).

If the first and second basophil engaging domains are a pair of VH and VL domains, the VH and VL domains may be specific for an antigen expressed on the surface of a basophil, such as CD89 (Fc alpha receptor 1) or FcεRI.

Table 11 presents selected publications on some exemplary antibodies specific for an antigen expressed on the surface of a basophil.

TABLE 11
Selected References Showing Specificity of Exemplary
Antibodies for Surface Antigens on Basophils

| CD89 (Fc alpha | Xu et al. Critical Role of Kupffer Cell CD89 Expression in |
|---|---|
| receptor 1) | Experimental IgA Nephropathy. PLoS One. 11(7): e0159426 (2016). |
| | Monteiro R C et al. IgA Fc receptors. Annu Rev Immunol. 21: 177- |
| | 204 (2003) |
| | Morton H C et al. CD89: the human myeloid IgA Fc receptor. |
| | Arch Immunol Ther Exp (Warsz). 49(3): 217-29 (2001) |
| FcεRI | Stone K D et al. IgE, mast cells, basophils, and eosinophils. |
| | J Allergy Clin Immunol. 125(2 Suppl 2): S73-80 (2010) |
| | Conner E R and Saini S S. The immunoglobulin E receptor: |
| | expression and regulation. Curr Allergy Asthma Rep. 5(3): 191-6 |
| | (2005) |

7. γδ T cells

In some embodiments, the immune cell engaging domain is a γδ T-cell engaging domain. As used herein, a γδ T cell refers to a T cell having a TCR made up of one gamma chain (γ) and one delta chain (δ).

When the two γδ T-cell engaging domains are associated together in the two-component system, they may bind to an antigen on the surface of the γδ T cell to engage these cells. In some embodiments, the antigen on the surface of the γδ T cell may be γδ TCR, NKG2D, CD3 Complex (CD3ε, CD3γ, CD3δ, CD3ζ, CD3ζ), 4-1BB, DNAM-1, or TLRs (e.g., TLR2, TLR6).

In some embodiments, having one half of the two-component system bind to a surface protein on the γδ T cell and having the other half of the system bind to cancer cells allows specific engagement of γδ T cells. Engagement of γδ T cell can lead to cytolysis of the target cell and release of proinflammatory cytokines such as TNFα and IFNγ.

When the two γδ T-cell engaging domains are associated together in the ATTAC, the γδ T cell may kill the target cell.

In some embodiments, the first γδ T-cell engaging domain is a VH domain and the second γδ T-cell engaging domain is a VL domain. In other embodiments, the first γδ T-cell engaging domain is a VL domain and the second γδ T-cell engaging domain is a VH domain. In such embodiments, when paired together the first and second γδ T-cell engaging domains may comprise an scFv (by this we mean equivalent to an scFv but for the fact that the VH and VL are not in a single-chain configuration).

If the first and second γδ T-cell engaging domains are a pair of VH and VL domains, the VH and VL domains may be specific for an antigen expressed on the surface of a γδ T cell, such as γδ TCR, NKG2D, CD3 Complex (CD3ε, CD3γ, CD3δ, CD3ζ, CD3η), 4-1BB, DNAM-1, or TLRs (TLR2, TLR6).

Table 12 presents selected publications on some exemplary antibodies specific for an antigen expressed on the surface of a γδ T cell.

TABLE 12
Selected References Showing Specificity of Exemplary Antibodies
for Surface Antigens on Gamma-delta (γδ) T cells

| | |
|---|---|
| γδ TCR | Vantourout P and Hayday A. Six-of-the-best: unique contributions |
| | of γδ T cells to immunology. Nat Rev Immunol. 13(2): 88-100 (2013) |
| | Hayday A and Tigelaar R. Immunoregulation in the tissues by |
| | gammadelta T cells. Nat Rev Immunol. 3(3): 233-42 (2003) |
| | Hayday A C. γδ cells: a right time and a right place for a conserved |
| | third way of protection. Annu Rev Immunol. 18: 975-1026 (2000) |
| NKG2D | Vantourout P and Hayday A. Six-of-the-best: unique contributions |
| | of γδ T cells to immunology. Nat Rev Immunol. 13(2): 88-100 (2013) |
| | Hayday A and Tigelaar R. Immunoregulation in the tissues by |
| | gammadelta T cells. Nat Rev Immunol. 3(3): 233-42 (2003) |
| | Hayday A C. γδ cells: a right time and a right place for a conserved |
| | third way of protection. Annu Rev Immunol. 18: 975-1026 (2000) |
| | Raulet D H et al. Regulation of ligands for the NKG2D activating |
| | receptor. Annu Rev Immunol. 31: 413-41 (2013) |
| CD3 | Vantourout P and Hayday A. Six-of-the-best: unique contributions |
| Complex | of γδ T cells to immunology. Nat Rev Immunol. 13(2): 88-100 (2013) |
| (CD3α, | Hayday A and Tigelaar R. Immunoregulation in the tissues by |
| CD3β, CD3γ, | gammadelta T cells. Nat Rev Immunol. 3(3): 233-42 (2003) |
| CD3γ, CD3ε) | Hayday A C. γδ cells: a right time and a right place for a conserved |
| | third way of protection. Annu Rev Immunol. 18: 975-1026 (2000) |
| 4-1BB | Ochoa M C et al. Antibody-dependent cell cytotoxicity: |
| | immunotherapy strategies enhancing effector NK cells. Immunol Cell |
| | Biol. 95(4): 347-355 (2017) |
| DNAM-1 | Niu C et al. Low-dose bortezomib increases the expression of |
| | NKG2D and DNAM-1 ligands and enhances induced NK and γδ T cell- |
| | mediated lysis in multiple myeloma. Oncotarget. 8(4): 5954-5964 |
| | (2017) |
| | Toutirais O et al. DNAX accessory molecule-1 (CD226) promotes |
| | human hepatocellular carcinoma cell lysis by Vgamma9Vdelta2 T cells. |
| | Eur J Immunol. 39(5): 1361-8 (2009) |
| TLRs (TLR2, | Beutler B A. TLRs and innate immunity. Blood. 113(7): 1399-407 |
| TLR6) | (2009) |
| | Beutler B et al. Genetic analysis of host resistance: Toll-like receptor |
| | signaling and immunity at large. Annu Rev Immunol. 24: 353-89 (2006) |

8. Natural Killer T Cells (NKT Cells)

In some embodiments, the immune cell engaging domain is a NKT engaging domain. NKT cells refers to T cells that express the Vα24 and Vβ11 TCR receptors.

When the two NKT engaging domains are associated together in the two-component system, they may bind to an antigen on the surface of the NKT to engage these cells. In some embodiments, the antigen on the surface of the NKT may be αβTCR, NKG2D, CD3 Complex (CD3ε, CD3γ, CD3δ, CD3ζ, CD3η), 4-1BB, or IL-12R.

In some embodiments, having one half of the two-component system bind to a surface protein on the NKT and having the other half of the system bind to cancer cells allows specific engagement of NKT. Engagement of NKTs can lead to cytolysis of the target cell.

When the two NKT engaging domains are associated together in the ATTAC, the NKT may cytolysis of the target cell and the release of proinflammatory cytokines.

In some embodiments, the first NKT engaging domain is a VH domain and the second NKT engaging domain is a VL domain. In other embodiments, the first NKT engaging domain is a VL domain and the second NKT engaging domain is a VH domain. In such embodiments, when paired together the first and second NKT engaging domains may comprise an scFv (by this we mean equivalent to an scFv but for the fact that the VH and VL are not in a single-chain configuration).

If the first and second NKT engaging domains are a pair of VH and VL domains, the VH and VL domains may be specific for an antigen expressed on the surface of a NKT, such as αβTCR, NKG2D, CD3 Complex (CD3ε, CD3γ, CD3δ, CD3ζ, CD3η), 4-1BB, or IL-12R.

Table 13 presents selected publications on some exemplary antibodies specific for an antigen expressed on the surface of a NKT.

TABLE 13
Selected References Showing Specificity of Exemplary
Antibodies for Surface Antigens on NKT cells

| | |
|---|---|
| αβTCR | Courtney A H et al. TCR Signaling: Mechanisms of Initiation and |
| | Propagation. Trends Biochem Sci. 43(2): 108-123 (2018) |
| | Davis M M et al. Ligand recognition by alpha beta T cell receptors. |
| | Annu. Rev. Immunol. 16: 523-544 (1998) |
| NKG2D | Sentman C L and Meehan K R. NKG2D CARs as cell therapy for |
| | cancer. Cancer J. 20(2): 156-9 (2014) |
| | Ullrich E et al. New prospects on the NKG2D/NKG2DL system for |
| | oncology. Oncoimmunology. 2(10): e26097 (2013) |
| CD3 Complex (CD3α, | Courtney A H et al. TCR Signaling: Mechanisms of Initiation and |
| CD3β, CD3γ, | Propagation. Trends Biochem Sci. 43(2): 108-123 (2018) |
| CD3γ, CD3ε) | |
| 4-1BB | Makkouk A et al. Rationale for anti-CD137 cancer immunotherapy. |
| | Eur J Cancer. 54: 112-119 (2016) |
| | Zhou S J. Strategies for Bispecific Single Chain Antibody in Cancer |
| | Immunotherapy. J Cancer. 8(18): 3689-3696 (2017) |
| IL-12R | Lasek W et al. Interleukin 12: still a promising candidate for tumor |
| | immunotherapy? Cancer Immunol Immunother. 63(5): 419-35 (2014) |

9. Engineered Immune Cells

In some embodiments, the immune cell engaging domain is an engineered immune cell engaging domain.

In some embodiments, the engineered immune cell is a chimeric antigen receptor (CAR) cell. In some embodiments, the CAR comprises an extracellular domain capable of tightly binding to a tumor antigen (for example, an scFv), fused to a signaling domain partly derived from a receptor naturally expressed by an immune cell. Exemplary CARs are described in Facts about Chimeric Antigen Receptor (CAR) T-Cell Therapy, Leukemia and Lymphoma Society, December 2017. CARs may comprise an scFV region specific for a tumor antigen, an intracellular co-stimulatory domain, and linker and transmembrane region. For example, a CAR in a CAR T cell may comprise an extracellular domain of a tumor antigen fused to a signaling domain partly derived from the T cell receptor. A CAR may also comprise a co-stimulatory domain, such as CD28, 4-1 BB, or OX40. In some embodiments, binding of the CAR expressed by an immune cell to a tumor target antigen results in immune cell activation, proliferation, and target cell elimination. Thus, a range of CARs may be used that differ in their scFV region, intracellular co-stimulatory domains, and linker and transmembrane regions to generate engineered immune cells.

Exemplary engineered immune cells include CAR T cells, NK cells, NKT cells, and γδ cells. In some embodiments, engineered immune cells are derived from the patient's own immune cells. In some embodiments, the patient's tumor expresses a tumor antigen that binds to the scFV of the CAR.

Potential CAR targets studied so far include CD19, CD20, CD22, CD30, CD33, CD123, ROR1, Igk light chain, BCMA, LNGFR, and NKG2D. However, the CAR technology would be available for developing engineered immune cells to a range of tumor antigens.

In some embodiments, the engineered immune cell is a genetically engineered immune cell.

When the two engineered immune cell engaging domains are associated together in the two-component system, they may bind to an antigen on the surface of the engineered immune cell to engage these cells. In some embodiments, the antigen on the surface of the engineered immune cell may be be an engagement domain recited in this application with specificity for T cells, NK cells, NKT cells, or γδ cells.

In some embodiments, having one half of the two-component system bind to a surface protein on the engineered immune cell and having the other half of the system bind to cancer cells allows specific engagement of engineered immune cells. Engagement of engineered immune cells can lead to activation of the effector response of these cells such as cytolysis of their target and release of cytokines.

When the two engineered immune cell engaging domains are associated together in the ATTAC, the engineered immune cell may kill the target cell.

In some embodiments, the first engineered immune cell engaging domain is a VH domain and the second engineered immune cell engaging domain is a VL domain. In other embodiments, the first engineered immune cell engaging domain is a VL domain and the second engineered immune cell engaging domain is a VH domain. In such embodiments, when paired together the first and second engineered immune cell engaging domains may comprise an scFv (by this we mean equivalent to an scFv but for the fact that the VH and VL are not in a single-chain configuration).

If the first and second engineered immune cell engaging domains are a pair of VH and VL domains, the VH and VL domains may be specific for an antigen expressed on the surface of an engineered immune cell, based on the type of cell used for the engineering.

E. Inert Binding Partner

The ATTAC also comprises at least one inert binding partner capable of binding the immune cell engaging domain to which it binds and preventing it from binding to another immune engaging domain unless certain conditions occur. When an immune cell engaging domain is bound to the at least one inert binding partner, it does not possess immune cell engaging activity.

In other words, the at least one inert binding partner cripples the function of an immune engaging domain by blocking it from binding its complementary pair (the other immune cell engaging domain) and preventing the two domains from joining together to have immune cell engaging activity. As such, the inert binding partner binds to an immune cell engaging domain such that the immune cell engaging domain does not bind to the other immune cell engaging domain unless the inert binding partner is removed. By does not bind, the application does not exclude nonspecific binding or low levels of binding (for example, ≤1%, ≤5%, ≤10%).

In some embodiments, the first immune cell engaging domain is bound to an inert binding partner. The inert binding partner bound to the first immune cell engaging domain prevents the first immune cell engaging domain from binding to the second immune cell binding domain.

In some embodiments, the second immune cell engaging domain is bound to an inert binding partner. The inert binding partner bound to the second immune cell engaging domain prevents the second immune cell engaging domain from binding to the first immune cell binding domain.

In some embodiments, the first and the second immune cell engaging domain are both bound to an inert binding partner. The inert binding partners bound to the first and the second immune cell engaging domain prevents the two immune cell engaging domain from binding to each other.

In some embodiments, the inert binding partner binds specifically to the immune cell engaging domain.

In some embodiments, the at least one inert binding partner is a VH or VL domain. In some embodiments, when the immune cell engaging domain in the ATTAC is a VH domain, the inert binding partner may be a VL domain and when the first immune cell engaging domain is a VL domain, the inert binding partner may be a VH domain.

If a first component comprises a targeting moiety and a VL immune cell engaging domain and a VH inert binding partner, in some embodiments, the VH inert binding partner has an equilibrium dissociation constant for binding to the VL immune cell engaging domain, which is greater than the equilibrium dissociation constant of the VL immune cell engaging domain for its partner VH immune cell engaging domain in the second component. In some embodiments, the prior sentence is equally true when VH is switched for VL and vice versa.

It is believed that using the inert binding partner as a mispairing partner with the immune cell engaging domain in the construct results in constructs that are more stable and easier to manufacture. In some embodiments, both the first and second immune binding domains may be bound to an inert binding partner as described herein. In some embodiments, only one of the immune binding domains is bound to an inert binding partner.

1. Inactivated VH or VL Domains as Inert Binding Partners

In some embodiments when an immune cell engaging domain is a VH or VL domain, the inert binding partner has homology to a corresponding VL or VH domain that can pair with the immune cell binding domain to form a functional antibody and bind to an immune cell antigen. This immune cell antigen may be an antigen present on any immune cell, including a T cell, a macrophage, a natural killer cell, a neutrophil, eosinophil, basophil, γδ T cell, natural killer T cell (NKT cells), or engineered immune cell. In some embodiments, this immune cell antigen is CD3.

In some embodiments, the inert binding partner is a VH or VL that cannot specifically bind an antigen when paired with its corresponding VL or VH of the immune cell engaging domain because of one or more mutations made in the inert binding partner to inhibit binding to the target antigen. In some embodiments, the VH or VL of the inert binding partner may differ by one or more amino acids from a VH or VL specific for an immune cell antigen. In other words, one or more mutations may be made to a VH or VL specific for a target immune cell antigen to generate an inert binding partner.

These mutations may be, for example, a substitution, insertion, or deletion in the polypeptide sequence of a VH or VL specific for an immune cell antigen to generate an inert binding partner. In some embodiments, the mutation in a VH or VL specific for an immune cell antigen may be made within CDR1, CDR2, or CDR3 to generate an inert binding partner. In some embodiments, an VH or VL used as an inert binding partner may retain the ability to pair with an immune cell engaging domain, but the resulting paired VH/VL domains have reduced binding to the immune cell antigen. In some embodiments, an inert binding partner has normal affinity to bind its corresponding immune cell engaging domain, but the paired VH/VL has lower binding affinity for the immune cell antigen compared to a paired VH/VL that does not comprise the mutation of the inert binding partner. For example, this lower affinity may be a 20-fold, 100-fold, or 1000-fold lower binding to an immune cell antigen.

In some embodiments, the first immune cell binding domain is a VH specific for an immune cell antigen and the inert binding partner is a VL domain for the same antigen that has one or more mutations such that the paired VH/VL has decreased or no binding to the antigen. In some embodiments, the first immune cell binding domain is a VL specific for an immune cell antigen and the inert binding partner is a VH domain for the same antigen that has one or more mutations such that the paired VH/VL has decreased or no binding to the antigen.

In some embodiments, the second immune cell binding domain is a VH specific for an immune cell antigen and the inert binding partner is a VL domain for the same antigen that has one or more mutations such that the paired VH/VL has decreased or no binding to the antigen. In some embodiments, the second immune cell binding domain is a VL specific for an immune cell antigen and the inert binding partner is a VH domain for the same antigen that has one or more mutations such that the paired VH/VL has decreased or no binding to the antigen.

2. Inert Binding Partners Obtained from Unrelated Antibodies

In some embodiments, a VH or VL used as an inert binding partner is unrelated to the VL or VH of the immune cell engaging domain. In other words, the inert binding partner may have little or no sequence homology to the corresponding VH or VL that normally associates with the VL or VH of the immune cell engaging domain. In some embodiments, the VH or VL used as an inert binding partner may be from a different antibody or scFv than the VL or VH used as the immune cell engaging domain.

If both components have inert binding partner, in some embodiments, the VH inert binding partner of one component and the VL inert binding partner of the other component may be from different antibodies.

F. Cleavage Site

By way of overview, the cleavage site may be (i) cleaved by an enzyme expressed by the cancer cells; (ii) cleaved through a pH-sensitive cleavage reaction inside the cancer cell; (iii) cleaved by a complement-dependent cleavage reaction; or (iv) cleaved by a protease that is colocalized to the cancer cell by a targeting moiety that is the same or different from the targeting moiety in the agent. In some embodiments, the cleavage site is a protease cleavage site.

The cleavage sites function to release the inert binding partner from the first immune cell engaging domain. The cleavage sites can function in different ways to release the inert binding partner from one or both immune cell engaging domains in the microenvironment of the cancer cells. The cleavage may occur inside the cancer cell or outside the cancer cell, depending on the strategy employed. If cleavage occurs outside the cancer cell, the immune cell engaging domain can be presented without first being internalized into a cell and being engaged in the classical antigen-processing pathways.

In certain embodiments, at least one cleavage site may be cleaved by an enzyme expressed by the cancer cells. Cancer cells, for instance, are known to express certain enzymes, such as proteases, and these may be employed in this strategy to cleave the ATTAC's one or more cleavage site. By way of nonlimiting example, cathepsin B cleaves FR, FK, VA and VR amongst others; cathepsin D cleaves PRSFFRLGK (SEQ ID NO: 45), ADAM28 cleaves KPAKFFRL (SEQ ID NO: 1), DPAKFFRL (SEQ ID NO: 2), KPMKFFRL (SEQ ID NO: 3) and LPAKFFRL (SEQ ID NO: 4); and MMP2 cleaves AIPVSLR (SEQ ID NO: 46), SLPLGLWAPNFN (SEQ ID NO: 47), HPVGLLAR (SEQ ID NO: 48), GPLGVRGK (SEQ ID NO: 49), and GPLGLWAQ (SEQ ID NO: 50), for example. Other cleavage sites listed in Table 1A or 3A may also be employed. Protease cleavage sites and proteases associated with cancer are well known in the art. Oncomine (www.oncomine.org) is an online cancer gene expression database, so when the agent of the invention is for treating cancer, the skilled person may search the Oncomine database to identify a particular protease cleavage site (or two protease cleavage sites) that will be appropriate for treating a given cancer type. Alternative databases include the European Bioinformatic Institute (www.ebi.ac.uk), in particular (www.ebi.ac.uk/gxa). Protease databases include ExPASy Peptide Cutter (ca.expasy.org/tools/peptidecutter) and PMAP.Cut DB (cutdb.burnham.org).

In some embodiments, at least one cleavage site may be cleaved through a pH-sensitive cleavage reaction inside the cancer cell. If the ATTAC is internalized into the cell, the cleavage reaction may occur inside the cell and may be triggered by a change in pH between the microenvironment outside the cancer cell and the interior of the cell. Specifically, some cancer types are known to have acidic environments in the interior of the cancer cells. Such an approach may be employed when the interior cancer cell type has a characteristically different pH from the extracellular microenvironment, such as particularly the glycocalyx. Because pH cleavage can occur in all cells in the lysozymes, selection of a targeting agent when using a pH-sensitive cleavage site may require, when desired, more specificity. For example, when a pH-sensitive cleavage site is used, a targeting agent that binds only or highly preferably to cancer cells may be desired (such as, for example, an antibody binding to mesothelin for treatment of lung cancer).

In certain embodiments, at least one cleavage site may be cleaved by a complement-dependent cleavage reaction. Once the ATTAC binds to the cancer cell, the patient's complement cascade may be triggered. In such a case, the complement cascade may also be used to cleave the inert binding partner from the first immune cell engaging domain by using a cleavage site sensitive to a complement protease. For example, C1r and C1s and the C3 convertases (C4B,2a and C3b,Bb) are serine proteases. C3/C5 and C5 are also complement proteases. Mannose-associated binding proteins (MASP), serine proteases also involved in the complement cascade and responsible for cleaving C4 and C2 into C4b2b (a C3 convertase) may also be used. For example, and without limitation, C1s cleaves YLGRSYKV and MQLGRX. MASP2 is believed to cleave SLGRKIQI. Complement component C2a and complement factor Bb are believed to cleave GLARSNLDE.

In some embodiments, at least one cleavage site may be cleaved by a protease that is colocalized to the cancer cell by a targeting moiety that is the same or different from the targeting moiety in the ATTAC. For example, any protease may be simultaneously directed to the microenvironment of the cancer cells by conjugating the protease to a targeting agent that delivers the protease to that location. The targeting agent may be any targeting agent described herein. The protease may be affixed to the targeting agent through a peptide or chemical linker and may maintain sufficient enzymatic activity when bound to the targeting agent.

In some embodiments, both the first component and second component are mispaired with an inert binding partner. In some embodiments, the protease cleavage site in the first component and the second component are the same. In other embodiments, the protease cleavage sites in the first component and the second component are different cleavage sites for the same protease. In other embodiments, the protease cleavage sites in the first component and the second component are cleavage sites for different proteases. In some embodiments employing two different proteases, the cancer cell expresses both proteases.

In some embodiments, in a first component, the inert binding partner in an uncleaved state interferes with the specific binding of a VL or VH immune engaging domain to its partner VH or VL, respectively, immune cell engaging domain in a second component. In some embodiments, the inert binding partner in an uncleaved state inhibits the binding of the VL or VH immune cell engaging domain to its partner VH or VL, respectively, immune cell engaging domain in a second component such that the dissociation constant (Kd) of the VL or VH immune cell engaging domain to its partner VH or VL, respectively, immune cell engaging domain in a second component in an uncleaved state is at least 100 times greater than the Kd of the VL or VH immune cell engaging domain to its partner VH or VL, respectively, immune cell engaging domain in a second component in a cleaved state.

G. Linkers

In addition to the cleavage site, linkers may optionally be used to attach the separate parts of the ATTAC together. By linker, we include any chemical moiety that attaches these parts together. In some embodiments, the linkers may be flexible linkers. Linkers include peptides, polymers, nucleotides, nucleic acids, polysaccharides, and lipid organic species (such as polyethylene glycol). In some embodiments, the linker is a peptide linker. Peptide linkers may be from about 2-100, 10-50, or 15-30 amino acids long. In some embodiments, peptide linkers may be at least 10, at least 15, or at least 20 amino acids long and no more than 80, no more than 90, or no more than 100 amino acids long. In some embodiments, the linker is a peptide linker that has a single or repeating GGGGS (SEQ ID NO: 85), GGGS (SEQ ID NO: 86), GS (SEQ ID NO: 87), GSGGS (SEQ ID NO: 88), GGSG (SEQ ID NO: 89), GGSGG (SEQ ID NO: 90), GSGSG (SEQ ID NO: 91), GSGGG (SEQ ID NO: 92), GGGSG (SEQ ID NO: 93), and/or GSSSG (SEQ ID NO: 94) sequence(s).

In some embodiments, the linker is a maleimide (MPA) or SMCC linker.

H. Methods of Making

The ATTACs as described herein can be made using genetic engineering techniques. Specifically, a nucleic acid may be expressed in a suitable host to produce an ATTAC. For example, a vector may be prepared comprising a nucleic acid sequence that encodes the ATTAC including all of its component parts and linkers and that vector may be used to transform an appropriate host cell.

Various regulatory elements may be used in the vector as well, depending on the nature of the host and the manner of introduction of the nucleic acid into the host, and whether episomal maintenance or integration is desired.

Chemical linkage techniques, such as using maleimide or SMCC linkers, may also be employed.

In instances where the binding partner is an aptamer, a person of ordinary skill in the art would appreciate how to conjugate an aptamer to a protein, namely the immune cell engaging domain. Aptamers may be conjugated using a thiol linkage or other standard conjugation chemistries. A maleimide, succinimide, or SH group may be affixed to the aptamer to attach it to the immune cell engaging domain.

### II. Pharmaceutical Compositions

The ATTACs may be employed as pharmaceutical compositions. As such, they may be prepared along with a pharmaceutically acceptable carrier. If parenteral administration is desired, for instance, the ATTACs may be provided in sterile, pyrogen-free water for injection or sterile, pyrogen-free saline. Alternatively, the ATTACs may be provided in lyophilized form for resuspension with the addition of a sterile liquid carrier.

### III. Methods of Using ATTACs

The ATTACs described herein may be used in a method of treating a disease in a patient characterized by the presence of cancer cells comprising administering an ATTAC comprising at least a first and a second component to the patient, as each of the components have been described in detail in various embodiments above. Additionally, the agents described herein may also be used in a method of targeting a patient's own immune response to cancer cells comprising administering an ATTAC to the patient.

In some embodiments, the patient has cancer or a recognized pre-malignant state. In some embodiments, the patient has undetectable cancer, but is at high risk of developing cancer, including having a mutation associated with an increased risk of cancer. In some embodiments, the patient at high risk of developing cancer has a premalignant tumor with a high risk of transformation. In some embodiments, the patient at high risk of developing cancer has a genetic profile associated with high risk. In some embodiments, the presence of cancer or a pre-malignant state in a patient is determined based on the presence of circulating tumor DNA (ctDNA) or circulating tumor cells. In some embodiments, treatment is pre-emptive or prophylactic. In some embodiments, treatment slow or blocks the occurrence or reoccurrence of cancer.

The amount of the agent administered to the patient may be chosen by the patient's physician so as to provide an effective amount to treat the condition in question. The first component and the second component of the ATTAC may be administered in the same formulation or two different formulations within a sufficiently close period of time to be active in the patient.

The patient receiving treatment may be a human. The patient may be a primate or any mammal. Alternatively, the patient may be an animal, such as a domesticated animal (for example, a dog or cat), a laboratory animal (for example, a laboratory rodent, such as a mouse, rat, or rabbit), or an animal important in agriculture (such as horses, cattle, sheep, or goats).

The cancer may be a solid or non-solid malignancy, The cancer may be any cancer such as breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, liver cancer, leukemia, myeloma, nonHodgkin lymphoma, Hodgkin lymphoma, acute myeloid leukemia, acute lymphoblastic leukemia, chronic lymphoblastic leukemia, lymphoproliferative disorder, myelodysplastic disorder, myeloproliferative disease and premalignant disease.

In some embodiments, a patient treated with an ATTAC has a tumor characterized by the presence of high levels of regulatory T cells (see Fridman W H et al., Nature Reviews Cancer 12:298-306 (2012) at Table 1). In patients with tumors characterized by a high presence of regulatory T cells, ATTAC therapy may be advantageous over other therapies that non-selectively target T cells, such as unselective BiTEs. In some embodiments, ATTAC therapy avoids engagement of regulatory T cells. In some embodiments, at least 80%, 85%, 90%, 91%, 92%, 93%, 94%, 95%, 96%, 97%, 98%, 99% of activated T cells are not regulatory T cells. In some embodiments, no regulatory T cells are activated by ATTAC therapy.

In some embodiments, the presence of a biomarker is used to select patients for receiving the ATTAC. A wide variety of tumor markers are known in the art, such as those described at www.cancer.gov/about-cancer/diagnosis-staging/diagnosis/tumor-markers-fact-sheet. In some embodiments, the tumor marker is ALK gene rearrangement or overexpression; alpha-fetoprotein; beta-2-microglobulin; beta-human chorionic gonadotropin; BRCA1 or BRCA2 gene mutations; BCR-ABL fusion genes (Philadelphia chromosome); BRAF V600 mutations; C-kit/CD117; CA15-3/CA27.29; CA19-9; CA-125; calcitonin; carcinoembryonic antigen (CEA); CD20; chromogranin A (CgA); chromosomes 3, 7, 17, or 9p21; circulating tumor cells of epithelial origin (CELLSEARCH®); cytokeratin fragment 21-1; EGFR gene mutation analysis; estrogen receptor (ER)/progesterone receptor (PR); fibrin/fibrinogen; HE4; HER2/neu gene amplification or protein overexpression; immunoglobulins; KRAS gene mutation analysis; lactate dehydrogenase; neuron-specific enolase (NSE); nuclear matrix protein 22; programmed death ligand 1 (PD-L1); prostate-specific antigen (PSA); thyroglobulin; urokinase plasminogen activator (uPA); plasminogen activator inhibitor (PAI-1); 5-protein signature (OVA1®); 21-gene signature (Oncotype DX®); or 70-gene signature (Mammaprint®).

The ATTAC may be administered alone or in conjunction with other forms of therapy, including surgery, radiation, traditional chemotherapy, or immunotherapy.

In some embodiments, the immunotherapy is checkpoint blockade. Checkpoint blockade refers to agents that inhibit or block inhibitory checkpoint molecules that suppress immune functions. In some embodiments, the checkpoint blockade targets CTLA4, PD1, PD-L1, LAG3, CD40, TIGIT, TIM3, VISTA or HLA-G.

In some embodiments, the immunotherapy is immune cytokines or cytokine fusions. Cytokines refer to cell-signaling proteins naturally made by the body to activate and regulate the immune system. Cytokine fusions refer to engineered molecules comprising all or part of a cytokine. For example, a cytokine fusion may comprise all or part of a cytokine attached to an antibody that allows targeting to a tumor such as Darleukin (see Zegers et al. (2015) Clin. Cancer Res., 21, 1151-60), Teleukin (see WO2018087172).

In some embodiments, the immunotherapy is cancer treatment vaccination. In some embodiments, cancer treatment vaccination boosts the body's natural defenses to fight cancer. These can either be against shared tumor antigens (such as E6, E7, NY-ESO, MUC1, or HER2) or against personalized mutational neoantigens.

### EXAMPLES

### Example 1: Labelling T Cells with ATTAC

To facilitate initial testing of the ATTAC platform and to show proof of concept, a model system employing FITC was used. Immune cells were stained with FITC-labelled antibodies against immune cell markers and anti-FITC ATTAC components were used for initial testing.

Thus, in this model, the anti-FITC ATTAC component (SEQ ID NO: 165) acts as an adapter ATTAC component whereby firstly, FITC-labelled antibodies can be used to label different target antigens on the immune cells of interest. Using an adapter ATTAC component means a large number of antigens on the immune cell surface can be assayed using one ATTAC component that constitutes half of the required two components. Immune cells would then be labelled with the anti-FITC ATTAC component, only if the FITC-labelled antibody bound to the cells of interest. The anti-FITC ATTAC component would contain one half of the immune cell activating domain with the second half of the immune cell activating domain coming from a second ATTAC component bound to an antigen on the unwanted tumor cells.

In this experiment, we counted T cells (4×10⁶) and washed twice in RPMI+10% NBS. Re-suspended T cells to 2.6×10⁶ per ml and added 95 μl to 15 ml Falcon tubes and added 5 μl FITC antibodies (do not add anything to untreated T cells), then incubated at room temperature for 30 minutes.

Washed off excess antibody by adding 5 mls media and spinning down. Removed supernatant and re-suspended cells in residual media (around 80 ul). Added 100 ul media to each tube.

Added 20 l anti-FITC ATTAC component (SEQ ID NO: 165-300 μg/ml) to each tube so there was a final concentration of 30 μg/ml—incubated at room temperature for 30 minutes.

Washed off excess ATTAC component by adding 5 mls media and spinning down. Removed supernatant and re-suspended cells to 0.3×10⁶ per ml and add 100 μl per well of 96 well U-bottom plate.

The T cells then were labelled with CD3-V_{L} (from the 20G6 anti-CD3 clone) through the anti-FITC ATTAC component.

### Example 2: Labelling Tumor Cells with ATTAC

The unwanted tumor cells are labelled with either a combination of ATTAC or T-cell engaging antibodies (TEAC) components that bind to EpCAM and once processed at the cell surface, will re-combine to produce a functional anti-CD3 activating domain. TEACs refer to a kit or composition wherein both components target to a cancer cell (see WO2017/087789). TEACs lack an immune cell selection moiety, which is comprised in an ATTAC. This pairing was used as a positive control as this pairing generates a T cell response by cytokine secretion.

To pair with the anti-FITC ATTAC component, the unwanted tumor cells were labelled with an ATTAC component that bound to EpCAM on the tumor cell and once processed at the cell surface expressed the corresponding CD3 domain to the anti-FITC ATTAC component so that once the T cells with the anti-FITC ATTAC component and the tumor cells with the anti-EpCAM ATTAC component are mixed together, there is a functional anti-CD3 VH-VL domain to activate the wanted subset of T cells. Counted MCF-7 cells (12×10⁶) and washed twice in RPMI+10% NBS.

Re-suspended in media so there are 300,000 cells per 160 μl and added 2.56 ml to two 15 ml Falcon tubes labelled (i) EpCAM VH TEAC component (SEQ ID NO: 166) and EpCAM VL TEAC component (SEQ ID NO: 167) (the components form a TEAC [used as a control] when both components target to the cancer cell and neither component contains an immune cell selection moiety) and (ii) EpCAM VH ATTAC component (SEQ ID NO: 166) only. Also added 160 ul to another two Falcon tubes (iii) BiTE labelled (SEQ ID NO: 168) and (iv) untreated.

Mixed 320 μl EpCAM-20G6 VL TEAC component (300 μg/ml) and 320 μl EpCAM-20G6 VH TEAC component (300 μg/ml) together and added 640 ul to tube (i). Added 320 ul EpCAM-20G6 VH ATTAC component (300 μg/ml) to tube (ii). Final concentration of each ATTAC/TEACcomponent was 30 μg/ml. Incubated at room temperature for 30 minutes.

Washed off excess ATTAC/TEAC component by adding 5 mls media and spinning down. Removed supernatant and re-suspended cells to 1×10⁶ per ml and added 100 ul per well already containing the T cells (see above).

In tube (i), tumor cells were labelled with TEAC components containing both VH and VL. In tube (ii), the tumor cells were only labelled with the EpCAM ATTAC component containing the VH domain of the anti-CD3 and this can complement the VL domain of the anti-CD3 which can be found on the T cells.

### Example 3: Controls

As a positive control, tumor cells were labelled with BiTE (SEQ ID NO: 168) to demonstrate that if a complete anti-CD3 molecule is on the surface of the tumor cell, T cells can become activated. As a negative control, T cells were incubated with untreated tumor cells to demonstrate that there is no T cell activation if there is no anti-CD3 molecules on the tumor cell surface.

For BiTE treated cells, added 20 μl BiTE (SEQ ID NO: 168-20 μg/ml). Final concentration of BiTE was 2 μg/ml. Incubated at room temperature for 30 minutes.

Washed off excess BiTE by adding 5 mls media and spinning down. Removed supernatant and re-suspended cells to 1×10⁶ per ml and add 100 ul per well.

For untreated target cells, nothing was added. Incubated at room temperature for 30 minutes.

Added 5 mls media and spun down. Removed supernatant and re-suspended cells to 1×10⁶ per ml and added 100 ul per well.

Incubated plate at 37° C. overnight and used 100 μl supernatant for IFN-gamma ELISA and then pool cells from triplicate wells and use for FACS staining.

### Example 4: IFN-Gamma ELISA

For the IFN-gamma ELISA assay, a kit from ThermoFisher (Cat #88-7316-77) was used.

Background of IFNγ Assays Generally: Expression of cytokine markers in vitro, such as IFNγ expression, is known to have a predictive value for T cell responses and, thus, predicts in vivo results. As described in Ghanekar et al., Clin Diag Lab Immunol j8(3):628-31 (2001), IFNγ expression in CD8+ T cells measured by cytokine flow cytometry (CFC) is a surrogate marker for the response of cytotoxic T lymphocytes. Ghanekar at 628. Prior work showed that there is a strong correlation between the expression of IFNγ by CD8+ T cells and the activity of CTL effector cells. Ghanekar at 630. Prior work shows that the use of data on IFNγ expression allows greater accuracy in assessing CD8+ T-cell responses in a clinical setting. Id. at 631. This demonstrates that the cytokine expression assays herein were known to have predictive value for in vivo and clinical responses. While the methods herein do not follow the exact method steps of Ghanekar because there are multiple ways to assess IFNγ expression, Ghanekar demonstrates that IFNγ expression is a proxy for T-cell activity.

### Example 5: Flow Cytometry

Cells were washed in 3 ml FACS buffer (PBS+2% serum) and the supernatant discarded. Cells were stained with antibodies against CD3, CD4, CD8 and CD69 (T cell activation marker) for 30 minutes. Excess antibody was washed off using FACS buffer. The cells were filtered prior to running on the flow cytometer.

### Example 6: Results

FIGS. 3A-3C provides results from selective T-cell activation from TEACs. This experiment demonstrates that labelling T cells with FITC-conjugated antibodies does not alter their ability to recognize the CD3 molecule on the tumor cell surface and become activated in response to it. Target cells will be bound by the EpCAM-CD3VH and EpCAM-CD3VL TEAC components (and therefore have both halves of the anti-CD3 molecule). As shown in FIG. 3A, as expected, the amount of IFN gamma release across all tests with the TEAC labelled tumor cells is very similar and therefore, there is no obvious inhibitory effects of the FITC-conjugated antibodies on the T cell surface, i.e., no blocking by bound antibody.

The controls worked well with strong T cell activation by BiTE and there is no T cell activation when they are incubated with unlabeled target cells (no anti-CD3 on the cell surface). Thus, more specifically, this control experiment shows that TEACs are not selective between CD4 and CD8 and that using an FITC model did not alter the expected results. The use of the FITC model does not prevent T cell activation. The results seen in FIG. 3A-C demonstrate the activation of all T cell subsets (CD4 and CD8) when there is a full anti-CD3 activating domain on the tumor cell.

FIGS. 3B and 3C demonstrate T cell activation by CD69 flow cytometry staining using the mean fluorescence intensity above background as readout. Similar to the IFN gamma results, the activation of CD4 T cells (FIG. 3B) again demonstrated that there was no inhibitory effect of antibody labelling the T cells. Similar results can be seen with the CD8 T cells (FIG. 3C).

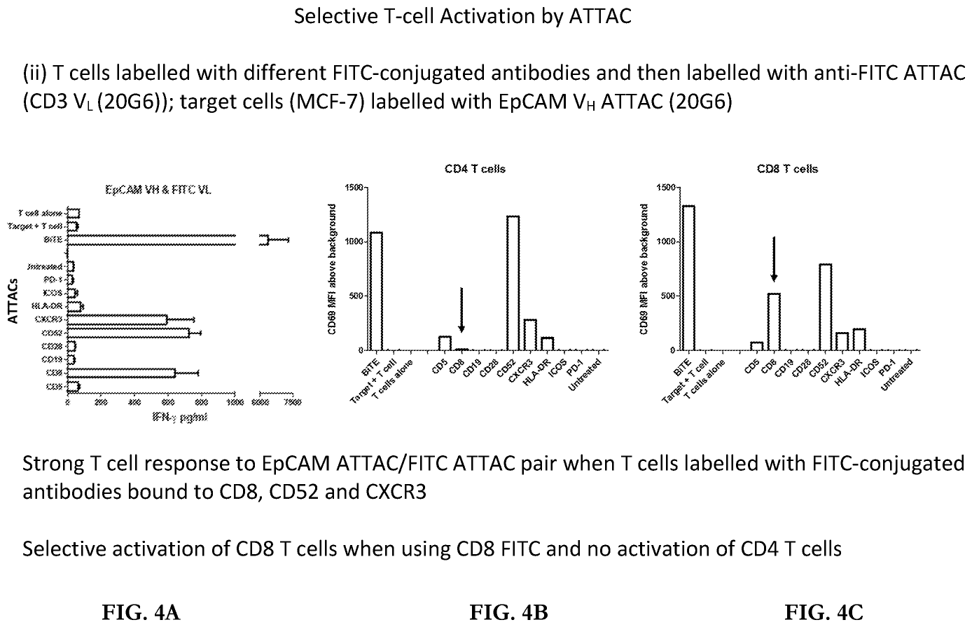

FIGS. 4A-C provides further evidence of selective T-cell activation by ATTACs. This part of the same experiment is a repeat of that in FIG. 3 but this time, the tumor cells only have one ATTAC component (EpCAM VH (SEQ ID NO: 166)); half of the anti-CD3 molecule) and the T cells have the anti-FITC ATTAC component (anti FITC VH (SEQ ID NO: 165)); the complementary half of the anti-CD3 molecule). When looking at the IFN Gamma results as a proxy for T cell activation (FIG. 4A), there is only T cell activation when T cells are labelled with CD52, CD8 and CXCR3. A strong T cell response to EpCAM ATTAC component/FITC ATTAC component pair was seen when when T cells labelled with FITC-conjugated antibodies bound to CD8, CD52 and CXCR3. FIGS. 4B (CD4 T cells) and FIG. 4C (CD8 T cells) demonstrate T cell activation by CD69 flow cytometry staining using the MFI above background as readout. Selective activation of CD8 T cells was seen when using anti-CD8 FITC ATTAC component, and there was no activation of CD4 T cells (see arrow in FIG. 4B versus FIG. 4C).

Therefore, even though all T cells express the listed proteins on their cell surface (see FIGS. 5A-5I), only binding the ATTAC component to CD52, CD8 and CXCR3 (via FITC) allowed T cell activation.

T cells stained with the FITC-conjugated antibodies prior to running the experiment to demonstrate that FITC will be on the T cell surface for the anti-FITC ATTAC component to bind to.

FIGS. 4B and 4C again demonstrate T cell activation by CD69 flow cytometry staining using the mean fluorescence intensity above background as readout. Both CD4 and CD8 T cells will express CD52, CD5, CXCR3 and HLA-DR. Therefore, the results that show activation of both CD4 and CD8 T cells labelled with these antibodies is expected and matches the results of the IFN gamma ELISA.

The results in FIGS. 4B and 4C with the CD8 labelling are the most important here as they demonstrate ATTACs can specifically activate one type of T cell over another. When all of the T cells are labelled with CD8-FITC ATTAC component and the anti-FITC ATTAC component, these proteins will only bind to the CD8 T cells and not the CD4 T cells. Once all T cells are incubated overnight with the tumor cells expressing the complementary ATTAC component, it can be seen from the flow cytometry that there is no activation of CD4 T cells but there is activation of CD8 T cells by CD69 staining.

Results in FIGS. 6A-8F use the same protocol as above and only differ from the experiment shown in FIGS. 3A-C and 4A-C by using freshly isolated unstimulated T cells prior to running the experiment and the addition of more FITC-conjugated antibodies for T cell labelling.

FIGS. 6A-6F provide additional evidence of selective T-cell activation by TEACs without blocking by FITC antibodies.

Target cells have both EpCAM-CD3VH and EpCAM-CD3VL (therefore have both halves of the anti-CD3 molecule). FIG. 6A shows, as expected, the amount of IFN gamma release across all tests with the EpCAM VH/VL TEAC pair-labelled tumor cells is very similar and therefore, there is no obvious inhibitory effects of the FITC-conjugated antibodies on the T cell surface, i.e., no blocking by bound antibody.

The controls in FIG. 6A have worked well with strong T cell activation by BiTE and there is no T cell activation when they are incubated with unlabeled target cells (no anti-CD3 on the cell surface).

FIGS. 6B-6E are representative raw data flow cytometry plots with FIG. 6F collating the T cell activation data for CD4 T cells. The plot in dashed line shows in FIGS. 6B-6E shows CD69 staining of untreated T cells that acts as a background level of CD69 activation. The plot in solid line in FIGS. 6B-6E shows the CD69 staining of T cells incubated overnight with the ATTAC labelled tumor cells. FIGS. 6B-6E present representative raw data flow cytometry plots with the collated data presented in FIG. 6F.

As expected, there is very similar CD4 T cell activation across all antibody labelled T cells as both TEAC components have been bound to the tumor cells.

FIGS. 7A-7F provide similar information as FIGS. 6A-F, but are directed to CD8 T cells. FIG. 7F shows similar CD8 T cell activation across all antibody labelled T cells as both TEAC components have been bound to the tumor cells. FIGS. 7B-7E present representative raw data flow cytometry plots with the collated data presented in FIG. 7F.

FIGS. 8A-8F offer additional information and are based on FIGS. 6A-6F and 7A-7F, but this time, the tumor cells are bound by one ATTAC component (half of the anti-CD3 molecule) and the T cells are bound by the anti-FITC ATTAC component (the complementary half of the anti-CD3 molecule). When looking at the IFN Gamma results as a proxy for T cell activation, there is only T cell activation when T cells are labelled with CD52, CD8 (four different anti-CD8 antibody clones) and CXCR3 (FIG. 8A). Again, FIGS. 8B-8E present representative raw data flow cytometry plots with the collated data presented in FIG. 8F.

Activation of CD4 T cells was only seen when bound with the CD52 and CXCR3 antibodies, and no activation of CD4 T cells was seen when bound with other antibodies including the CD8 antibodies.

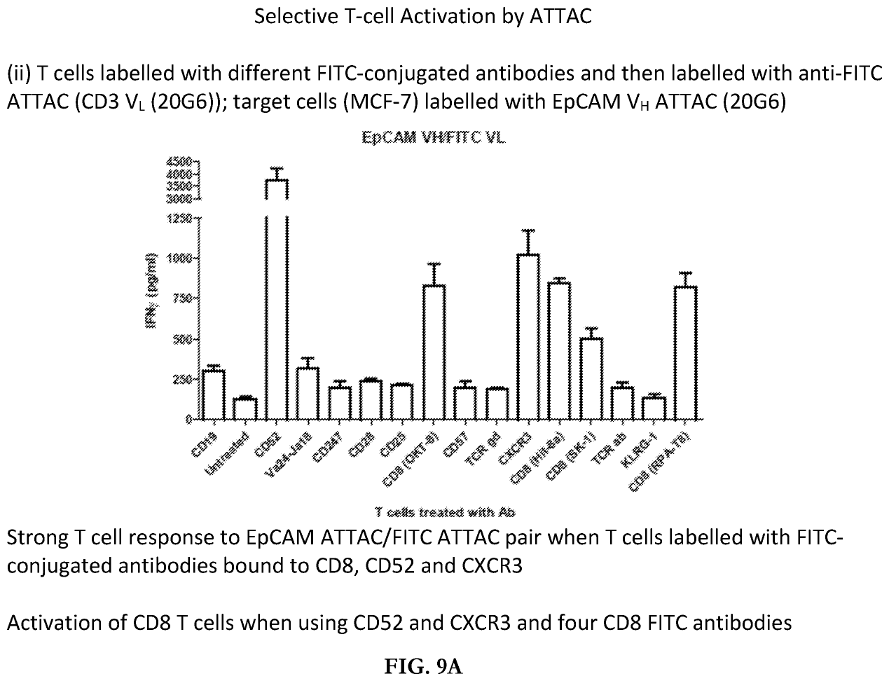

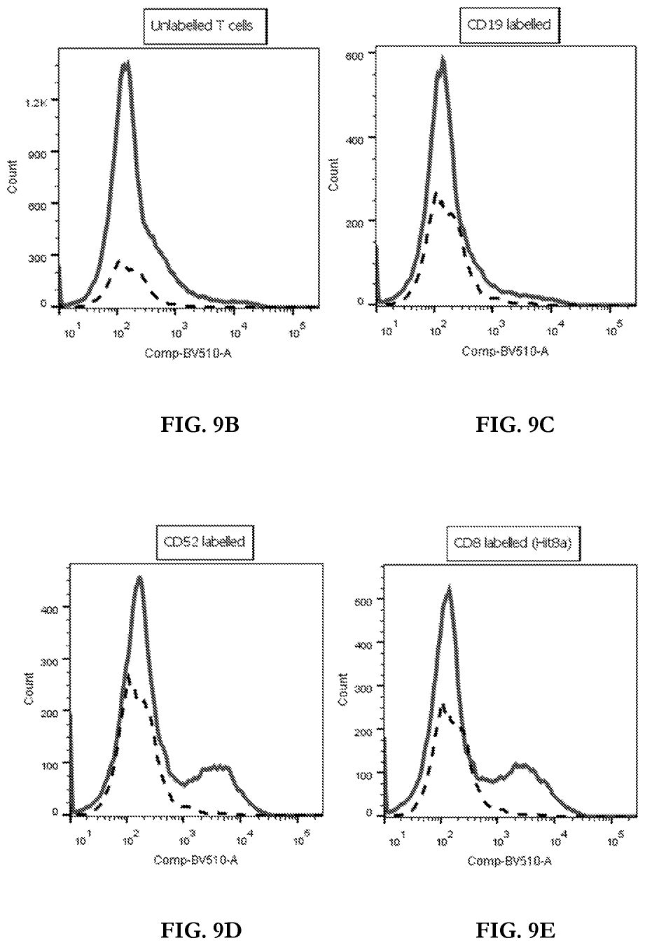

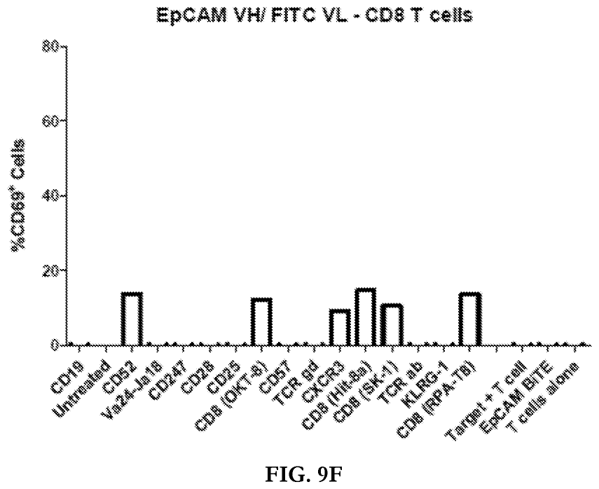

FIGS. 9A-9F provides a similar experiment to that shown in FIGS. 8A-8F, but for CD8 T cells. As shown in the collated data (FIG. 9F), CD52 and CXCR3 antibodies activated CD8 T cells in the same way they activate the CD4 T cells but this time, the CD8 antibodies activate the CD8 T cells as well.

These data support specific activation of CD8 T cells and not CD4 T cells using the CD8 FITC antibody and the anti-FITC ATTAC component as a means of getting the anti-CD3 VL on the T cell surface where it can pair with the anti-CD3 VH which is present on the tumor cell surface from binding of the EpCAM ATTAC component.

### Example 7. FACs Analysis Experiments Using Anti-CD8 ATTAC

Experiments were performed with direct targeting to immune cells, instead of using a model system employing FITC.

An ATTAC comprises two components. In these examples, for convenience, a first component comprising a targeted immune cell binding agent is referred to as an ATTAC1, and a second component comprising a selected immune cell binding agent is referred to as an ATTAC2.

In some experiments, a component that comprises a targeting moiety capable of targeting the cancer was used together with a second component that also comprises a targeting moiety capable of targeting the cancer to generate a TEAC. The TEACs are used herein as a control. The TEAC control shows activity induced when both components target the cancer cell.

MDA-MB-231 cells over-expressing EpCAM were labelled with anti-EpCAM ATTAC1 (containing the anti-CD3 VH domain (SEQ ID NO: 166)) and excess ATTAC component removed by washing.

Peripheral blood mononuclear cells (PBMCs) from healthy donor were labelled with the anti-CD8 ATTAC2 (containing the anti-CD3 VL domain (SEQ ID NO: 170)) and excess ATTAC component was removed by washing.

Control cells were labelled with anti-EpCAM TEACs. For experiments where anti-EpCAM TEACs were used (SEQ ID NOs: 166 and 167), both components will bind EpCAM on the tumor cells, without a targeting moiety that binds to an immune cell. In this control experiment, the TEAC pair thus will not confer specificity with an immune cell selection moiety.

The PBMCs were then co-cultured with the tumor cells at a PBMC to tumor cell ratio of 1:2. The ATTACs are proteolytically activatable by addition of an exogenous protease (enterokinase) with the protease added or not to the mixed cells. The co-cultured cells were then incubated overnight at 37° C.

After incubation, co-cultured cells were washed in FACS buffer (PBS+2% serum) and labelled for flow cytometry using CD3 APC-Cy7, CD4 PE, CD8 APC and CD69 FITC to ascertain the level of T cell activation (measured by an increase in CD69 staining) of the CD4 and CD8 T cell subsets.

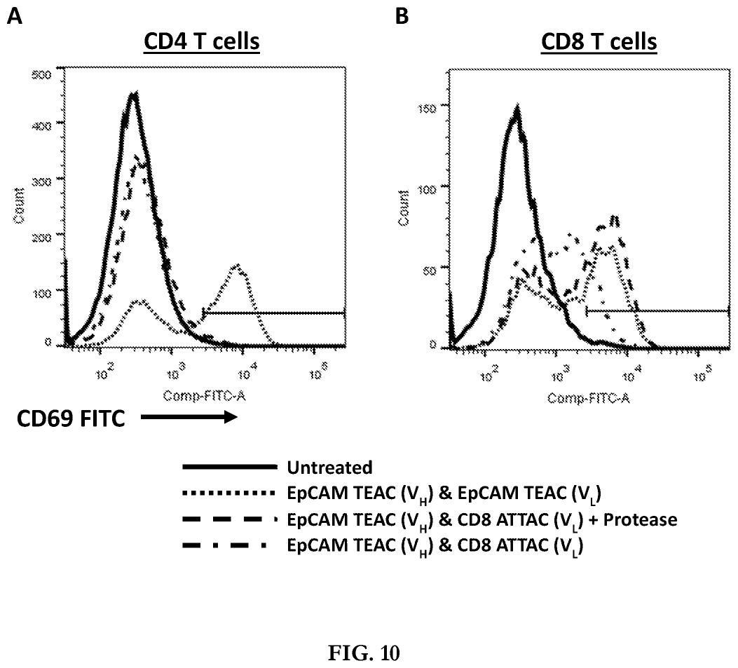

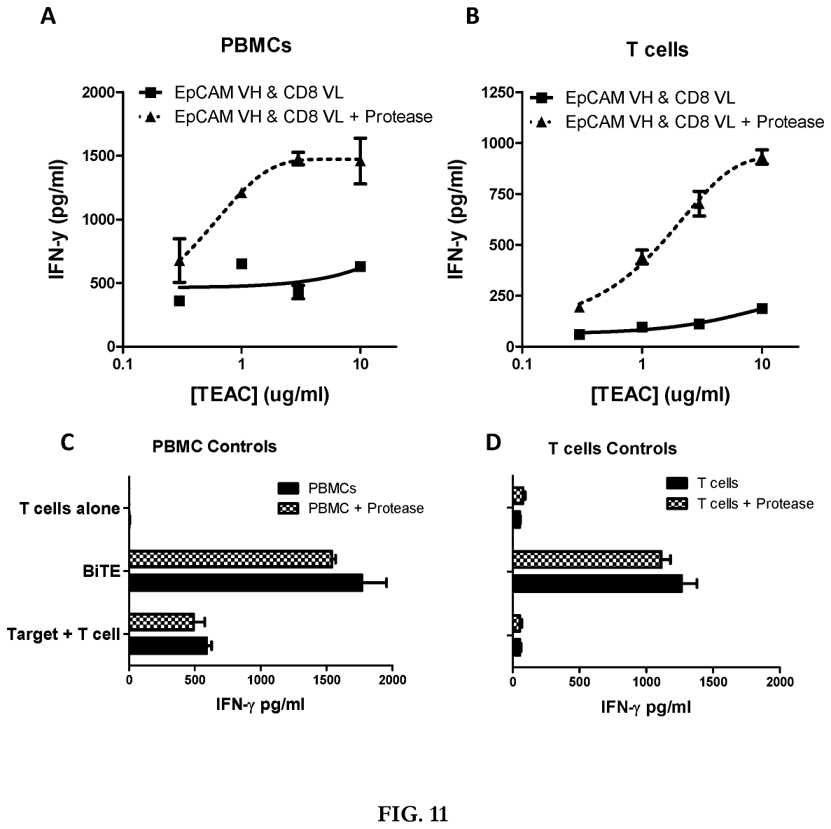

An increase in activation of CD8 T cells was seen after treatment with anti-EpCAM ATTAC1 and anti-CD8 ATTAC2 when enterokinase (protease) is added (FIG. 10B, dashed line). There was no activation for this ATTAC pair for labelled PBMCs without the addition of the exogenous protease (FIG. 10B, solid line) or the untreated PBMCs (filled histogram). These results confirm that ATTAC activity requires proteolytic activation. Furthermore, there is no activation of the CD4 T cell subset after treatment with anti-EpCAM ATTAC1 and anti-CD8 ATTAC2 in the presence of protease (FIG. 10A, dashed line), with results similar to the untreated PBMCs (filled histogram).

When both components of a TEAC are bound to the tumor cell (control wherein a TEAC component pair both bind to EpCAM) to form a functional anti-CD3 moiety at the tumor cell surface, both CD4 T cells (FIG. 10A, dotted line) and CD8 T cells (FIG. 10B, dotted line) are activated as measured by CD69 staining.

These results show that treatment with the EpCAM ATTAC VH (ATTAC1) plus CD8 ATTAC VL (ATTAC2) activates CD8 T cells in the presence of a protease, without activating CD4 T cells. In contrast, treatment with an EpCAM TEAC component pair activates both CD4 and CD8 T cells.

Thus, ATTACs can be used to specifically activate CD8 T cells, which are critical for successful anti-tumor immune responses.

### Example 8. Interferon Gamma Release Experiments Using Anti-CD8 ATTAC

Interferon gamma release was also used to evaluate activity of an ATTAC1 targeting a tumor cell antigen and an ATTAC2 targeting an immune cell antigen. In this example, ATTAC1 comprises a targeting moiety capable of targeting the cancer by targeting EpCAM expressed on the tumor cells and an anti-CD3 VH domain. ATTAC2 comprises an immune cell selection moiety capable of selectively targeting an immune cell by targeting CD8 and an anti-CD3 VH domain.

Tumor cells were labelled with increasing concentrations of anti-EpCAM ATTAC1 (containing both the an anti-EpCAM function and an anti-CD3 VH domain (SEQ ID NO: 166); termed “EpCAM VH”) and excess ATTAC component removed by washing. PBMCs from a healthy donor (FIG. 11A) or cultured T cells (FIG. 11B) were labelled with increasing concentration of the anti-CD8 ATTAC2 (containing both an anti-CD8 function and the anti-CD3 VL domain (SEQ ID NO: 170); termed “CD8 VL”), and excess ATTAC component was removed by washing. The PBMCs or T cells were then co-cultured with the tumor cells at a PBMC to tumor cell ratio of 1:4. The ATTACs were proteolytically activatable by addition of an exogenous protease (enterokinase) with the protease added or not to the mixed cells. The co-cultured cells were then incubated overnight at 37° C.

Following co-culture, the supernatant was assayed for the presence of interferon gamma (IFN-gamma), which denotes cytokine release by activated T cells. There was a dose-dependent increase in interferon gamma release by both PBMCs (FIG. 11A) and cultured T cells (FIG. 11B) when the cells are cultured in the presence of exogenous protease, but there is no increase in interferon gamma release when the protease is absent. The higher baseline levels of interferon gamma in the PBMCs compared to cultured T cells may be due to the presence of NK cells in the PBMC sample, as NK cells can produce interferon gamma.

The results in FIGS. 11A and 11B demonstrate the requirement of proteolytic activation of the ATTACs in generating a T cell response. Further, the ATTAC response was dose-dependent.

In the experimental controls, there was a lack of T cell activation, as measured by interferon gamma release, when T cells (FIG. 11D) or T cells in PBMC cultures (FIG. 11C) were cultured alone or with untreated tumor cells (target+T cell groups). As a positive control, T cells were activated when cultured with tumor cells labelled with an EpCAM-binding bi-specific T cell engager (BiTE; SEQ ID NO: 168).

### Example 9. Analysis of Concentration Dependence of ATTACs

The concentration dependence of an ATTAC pair was tested, wherein the ATTAC1 targeted a tumor cell antigen and an ATTAC2 targeted an immune cell antigen.

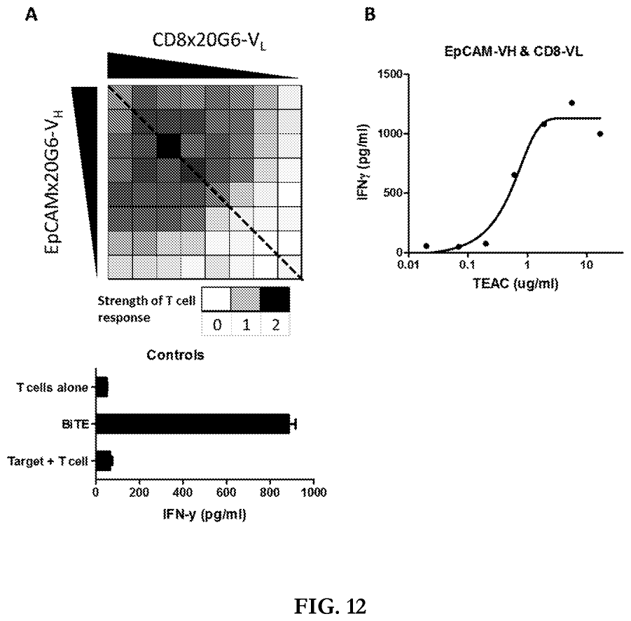

Tumor cells were labelled with increasing concentrations of anti-EpCAM ATTAC1 (containing the anti-CD3 VH domain; SEQ ID NO: 166) and excess ATTAC component removed by washing. PBMCs from a healthy donor (FIG. 12A) were labelled with increasing concentration of the anti-CD8 ATTAC2 (containing the anti-CD3 VL domain; SEQ ID NO: 170), and excess ATTAC component was removed by washing. Instead of keeping ATTAC1 and ATTAC2 at equimolar concentrations, the concentrations of ATTAC1 and ATTAC2 were at different molar concentrations to determine if there would be any skewing of T cell activation (by assaying for interferon gamma) towards one of the two ATTAC components. Once both the tumor cells and PBMCs had been labelled with the respective ATTAC components, the cells were co-cultured overnight at 37° C.

The data demonstrates strong T cell activation when the concentrations of ATTAC1 and 2 increase in equimolar concentrations (FIG. 12A). As the concentrations are skewed towards either ATTAC1 or ATTAC2, the level of T cell activation decreases, which suggests that the most potent activation of T cells (within PBMCs) is seen with equimolar concentrations of ATTAC1 and ATTAC2. FIG. 12B shows that increasing T cell activation with increasing equimolar concentrations of ATTAC1 and ATTAC2 (denoted by the dashed line in FIG. 12A) showed no skewing towards either ATTAC component used and that both ATTAC1 and ATTAC2 are equally important in activating T cells.

FIG. 12C shows control data for interferon release from T cells in PBMCs cultured alone or with untreated target cells. As a positive control, FIG. 12C shows strong interferon gamma release from T cells in PBMCs when cultured target cells were labelled by a BiTE (SEQ ID NO: 168).

### Example 10. Selective Activation of T Cell Subsets by ATTACs

Selective activation of T cell subsets was also tested using a model system employing FITC.

Tumor cells were labelled with an anti-EpCAM ATTAC1 (containing the anti-CD3 VH domain (SEQ ID NO: 166)), and excess ATTAC component was removed by washing. PBMCs from healthy donor were labelled with FITC-conjugated antibodies against CD4, CD8, or CD19 with excess antibody removed by washing. The PBMCs were further labelled with an anti-FITC ATTAC2 (containing the anti-CD3 VL domain (SEQ ID NO: 165)), and excess ATTAC component was removed by washing. The PBMCs were then co-cultured with the tumor cells at a PBMC to tumor cell ratio of 1:2. The ATTACs were proteolytically activatable by addition of an exogenous protease (enterokinase) with the protease added to the mixed cells. The co-cultured cells were then incubated overnight at 37° C.

In these experiments, FITC-labeled CD19 cells are a negative control, because CD19-expressing cells do not normally express CD3. Thus, binding of an anti-FITC ATTAC component to a CD19-positive cell would not lead to activation via a paired anti-CD3 VH/VL from an ATTAC component pair.

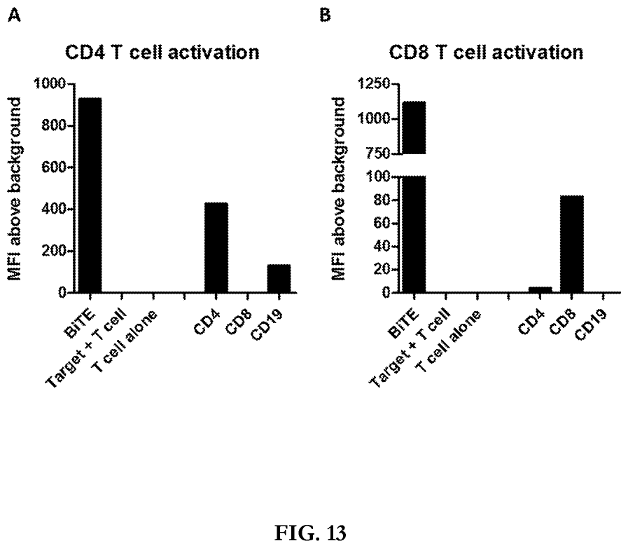

After incubation, co-cultured cells were washed in FACS buffer (PBS+2% serum) and labelled for flow cytometry using CD3 APC-Cy7, CD4 PE, CD8 APC and CD69 BV421 to ascertain the level of T cell activation (measured by the increase in CD69 staining) of CD4 and CD8 T cell subsets. Excess antibodies were removed by washing and the cells were analyzed by flow cytometry. CD4 T cells were only significantly activated (compared with the background activation of untreated T cells) when the PBMCs were labelled with the anti-CD4 FITC antibody (FIG. 13A). In this instance, the anti-FITC ATTAC2 containing the anti-CD3 VL domain would only bind to CD4 T cells, and this subset of T cells was activated. PBMCs labelled with the anti-CD8 or anti-CD19 FITC antibodies did not cause significant activation of the CD4 T cells, because CD4 cells do not express these antigens.

In contrast, CD8 T cells were only significantly activated (compared with the background activation of untreated T cells) when the PBMCs were labelled with the anti-CD8 FITC antibody (FIG. 13B). In this instance, the anti-FITC ATTAC2 containing the anti-CD3 VL domain would only bind to CD8 T cells, and this subset of T cells was activated. PBMCs labelled with the anti-CD4 or anti-CD19 FITC antibodies did not cause activation of the CD8 T cells, because CD8 cells do not express these antigens.

These data show the ability of ATTACs to activate a specific subset of T cell within a more complex mix of T cells. As shown FIGS. 13A and 13B, even using the same target cells and the same PBMCs, different subsets of immune cells could be activated using different ATTAC2 components.

Selective activation of a specific subset of immune cells could be therapeutically useful. For example, ATTACs that activate only cytotoxic T cells could avoid activation of unwanted T cells, such as regulatory T cells. Further, use of ATTACs that require cleavage by a tumor-associated protease can allow activation of immune cells within the tumor microenvironment. In this way, ATTACs could provide specificity for activating specific subsets of immune cells within the tumor microenvironment.

### Example 11. Prophetic ATTAC Experiments Using Anti-CD8 ATTAC Such as SEQ ID NO: 169 and 170

Peripheral blood mononuclear cells are labelled with the anti-CD8 ATTAC component and the excess ATTAC component removed by washing. The anti-CD8 ATTAC component contains one half of the anti-CD3 activating domain (VL). Unwanted tumor cell line would be labelled with an anti-EpCAM ATTAC component that contains the corresponding half of the anti-CD3 activating domain (VH) (SEQ ID NO: 166). The ATTAC would then be able to activate CD3 specifically on the CD8 T cells within the peripheral blood mononuclear cells. The activation of the CD8 T cells can be assayed by ELISA for IFN gamma secretion or by flow cytometry assaying for activation markers such as CD69 and CD38.

### Example 12. Prophetic ATTAC Experiments Using Anti-CD4 ATTAC Such as SEQ ID NO: 171

Peripheral blood mononuclear cells are labelled with the anti-CD4 ATTAC component and the excess ATTAC component removed by washing. The anti-CD4 ATTAC component contains one half of the anti-CD3 activating domain (VL) (SEQ ID NO: 166). Unwanted tumor cell line would be labelled with an anti-EpCAM ATTAC component that contains the corresponding half of the anti-CD3 activating domain (VH). The ATTAC would then be able to activate CD3 specifically on the CD4 T cells within the peripheral blood mononuclear cells. The activation of the CD4 T cells can be assayed by ELISA for IFN gamma secretion or by flow cytometry assaying for activation markers such as CD69 and CD38.

### Example 13. Embodiments

The following numbered items provide embodiments as described herein, though the embodiments recited here are not limiting.

Item 1. An agent for treating cancer in a patient comprising:

a. a first component comprising a targeted immune cell binding agent comprising:
i. a targeting moiety capable of targeting the cancer;
ii. a first immune cell engaging domain capable of immune engaging activity when binding a second immune cell engaging domain, wherein the second immune cell engaging domain is not part of the first component;

b. a second component comprising a selective immune cell binding agent comprising:
i. an immune cell selection moiety capable of selectively targeting an immune cell;
ii. a second immune cell engaging domain capable of immune cell engaging activity when binding the first immune cell engaging domain, wherein the first and second immune cell engaging domains are capable of binding when neither is bound to an inert binding partner,
wherein at least one of the first immune cell engaging domain or the second immune cell engaging domain is bound to an inert binding partner such that the first and second immune cell engaging domains are not bound to each other unless the inert binding partner is removed; and further comprising a cleavage site separating an inert binding partner and the immune cell engaging domain to which it binds, wherein the cleavage site is:
   1. cleaved by an enzyme expressed by the cancer cells;
   2. cleaved through a pH-sensitive cleavage reaction inside the cancer cell;
   3. cleaved by a complement-dependent cleavage reaction; or
   4. cleaved by a protease that is colocalized to the cancer cell by a targeting moiety that is the same or different from the targeting moiety in the agent.

Item 2. The agent of item 1, wherein the first component is not covalently bound to the second component.

Item 3. The agent of item 1, wherein the first component is covalently bound to the second component.

Item 4 The agent of any one of items 1-3, wherein the immune cell engaging domains, when bound to each other, are capable of binding an antigen expressed on the surface of the immune cell.

Item 5. The agent of any one of items 1-4, wherein the immune cell selection moiety capable of selectively targeting an immune cell selectively targets a T cell, a macrophage, a natural killer cell, a neutrophil, an eosinophil, a basophil, a γδ T cell, a natural killer T cell (NKT cells), or an engineered immune cell.

Item 6. The agent of item 5, wherein the immune cell selection moiety capable of selectively targeting an immune cell selectively targets a T cell.

Item 7. The agent of item 6, wherein the T cell is a cytotoxic T cell.

Item 8. The agent of item 7, wherein the cytotoxic T cell is a CD8+ T cell.

Item 9. The agent of item 6, wherein the T cell is a helper T cell.

Item 10. The agent of item 9, wherein the helper T cell is a CD4+ T cell.

Item 11. The agent of any one of items 6-10, wherein the immune cell selection moiety targets CD8, CD4, or CXCR3.

Item 12. The agent of any one of items 6-11, wherein the immune cell selection moiety does not specifically bind regulatory T cells.

Item 13. The agent of any one of items 6-12, wherein the immune cell selection moiety does not specifically bind TH17 cells.

Item 14. The agent of any one of items 6-13, wherein the immune cell engaging domains, when bound to each other, are capable of binding CD3.

Item 15. The agent of any one of items 6-13, wherein the immune cell engaging domains, when bound to each other, are capable of binding TCR.

Item 16. The agent of item 5, wherein the immune cell selection moiety capable of selectively targeting an immune cell selectively targets a natural killer cell.

Item 17. The agent of item 16, wherein the immune cell selection moiety targets CD2 or CD56.

Item 18. The agent of any one of items 16-17, wherein the immune cell engaging domains, when bound to each other, are capable of binding NKG2D, CD16, NKp30, NKp44, NKp46 or DNAM.

Item 19. The agent of item 5, wherein the immune cell selection moiety capable of selectively targeting an immune cell selectively targets a macrophage.

Item 20. The agent of item 19, wherein the immune cell selection moiety targets CD14, CD11b, or CD40.

Item 21. The agent of any one of items 19-20, wherein the immune cell engaging domains, when bound to each other, are capable of binding CD89 (Fc alpha receptor 1), CD64 (Fc gamma receptor 1), CD32 (Fc gamma receptor 2A) or CD16a (Fc gamma receptor 3A).

Item 22. The agent of item 5, wherein the immune cell selection moiety capable of selectively targeting an immune cell selectively targets a neutrophil.

Item 23. The agent of item 22, wherein the immune cell selection moiety targets CD15.

Item 24. The agent of any one of items 22-23, wherein the immune cell engaging domains, when bound to each other, are capable of binding CD89 (FcαR1), FcγRI (CD64), FcγRIIA (CD32), FcγRIIIA (CD16a), CD11b (CR3, αMβ2), TLR2, TLR4, CLEC7A (Dectin1), formyl peptide receptor 1 (FPR1), formyl peptide receptor 2 (FPR2), or formyl peptide receptor 3 (FPR3).

Item 25. The agent of item 5, wherein the immune cell selection moiety capable of selectively targeting an immune cell selectively targets an eosinophil.

Item 26. The agent of item 25, wherein the immune cell selection moiety targets CD193, Siglec-8, or EMR1.

Item 27. The agent of any one of items 25-26, wherein the immune cell engaging domains, when bound to each other, are capable of binding CD89 (Fc alpha receptor 1), FcεRI, FcγRI (CD64), FcγRIIA (CD32), FcγRIIIB (CD16b), or TLR4.

Item 28. The agent of item 5, wherein the immune cell selection moiety capable of selectively targeting an immune cell selectively targets a basophil.

Item 29. The agent of item 28, wherein the immune cell selection moiety targets 2D7, CD203c, or FcεRIα.

Item 30. The agent of any one of items 28-29, wherein the immune cell engaging domains, when bound to each other, are capable of binding CD89 (Fc alpha receptor 1) or FcεRI.

Item 31. The agent of item 5, wherein the immune cell selection moiety capable of selectively targeting an immune cell selectively targets a γδ T cell.

Item 32. The agent of item 31, wherein the immune cell selection moiety targets γδ TCR.

Item 33. The agent of any one of items 31-32, wherein the immune cell engaging domains, when bound to each other, are capable of binding γδ TCR, NKG2D, CD3 Complex (CD3ε, CD3γ, CD3δ, CD3ζ, CD3η), 4-1BB, DNAM-1, or TLRs (TLR2, TLR6).

Item 34. The agent of item 5, wherein the immune cell selection moiety capable of selectively targeting an immune cell selectively targets a natural killer T cell.

Item 35. The agent of item 34, wherein the immune cell selection moiety targets Va24 or CD56.

Item 36. The agent of any one of items 34-35, wherein the immune cell engaging domains, when bound to each other, are capable of binding αβTCR, NKG2D, CD3 Complex (CD3ε, CD3γ, CD3δ, CD3ζ, CD3η), 4-1BB, or IL-12R.

Item 37. The agent of item 5, wherein the immune cell selection moiety capable of selectively targeting an immune cell selectively targets an engineered immune cell.

Item 38. The agent of item 37, wherein the engineered immune cell is a chimeric antigen receptor (CAR) T cell, natural killer cell, natural killer T cell, or γδ T cell.

Item 39. The agent of item 37-38, wherein the immune cell selection moiety targets the CAR or a marker expressed on the immune cell.

Item 40. The agent of item 37-39, wherein the immune selection moieties targets LNGFR or CD20.

Item 41. The agent of item 37-40, wherein the immune cell engaging domains, when bound to each other, are capable of binding an antigen expressed by the engineered immune cell.

Item 42. The agent of item 37-41, wherein the antigen expressed by the engineered immune cell is CD3.

Item 43. The agent of any one of items 1-42, wherein the immune cell selection moiety comprises an antibody or antigen-specific binding fragment thereof.

Item 44. The agent of item 43, wherein the antibody or antigen-specific binding fragment thereof specifically binds an antigen on a T cell.

Item 45. The agent of any one of items 43, wherein the antibody or antigen-specific binding fragment thereof specifically binds an antigen on a cytotoxic or helper T cell.

Item 46. The agent of item 43, wherein the antibody or antigen-specific binding fragment thereof specifically binds an antigen on a macrophage.

Item 47. The agent of item 43, wherein the antibody or antigen-specific binding fragment thereof specifically binds an antigen on a natural killer cell.

Item 48. The agent of item 43, wherein the antibody or antigen-specific binding fragment thereof specifically binds an antigen on a neutrophil.

Item 49. The agent of item 43, wherein the antibody or antigen-specific binding fragment thereof specifically binds an antigen on an eosinophil.

Item 50. The agent of item 43, wherein the antibody or antigen-specific binding fragment thereof specifically binds an antigen on a γδ T cell.

Item 51. The agent of item 43, wherein the antibody or antigen-specific binding fragment thereof specifically binds an antigen on a natural killer T cell.

Item 52. The agent of item 43, wherein the antibody or antigen-specific binding fragment thereof specifically binds an antigen on an engineered immune cell.

Item 53. The agent of item 43, wherein the engineered immune cell is a CAR T cell, natural killer cell, natural killer T cell, or γδ T cell.

Item 54. The agent of any one of items 1-42, wherein the immune selection moiety comprises an aptamer.

Item 55. The agent of item 54, wherein the aptamer specifically binds an antigen on a T cell.

Item 56. The agent of item 55, wherein T cell is a cytotoxic or helper T cell.

Item 57. The agent of item 54, wherein the aptamer specifically binds an antigen on a macrophage.

Item 58. The agent of item 54, wherein the aptamer specifically binds an antigen on a natural killer cell.

Item 59. The agent of item 54, wherein the aptamer specifically binds an antigen on a neutrophil.

Item 60. The agent of item 54, wherein the aptamer specifically binds an antigen on an eosinophil.

Item 61. The agent of item 54, wherein the aptamer specifically binds an antigen on a γδ T cell.

Item 62. The agent of item 54, wherein the aptamer specifically binds an antigen on a natural killer T cell.

Item 63. The agent of item 54, wherein the aptamer specifically binds an antigen on an engineered immune cell.

Item 64. The agent of item 54, wherein the engineered immune cell is a CAR T cell, natural killer cell, natural killer T cell, or γδ T cell.

Item 65. The agent of any one of items 54-64, wherein the aptamer comprises DNA.

Item 66. The agent of any one of items 54-64, wherein the aptamer comprises RNA.

Item 67. The agent of any one of items 65-66, wherein the aptamer is single-stranded.

Item 68. The agent of any one of items 54-67, wherein the aptamer is a selective immune cell binding-specific aptamer chosen from a random candidate library.

Item 69. The agent of any one of items 1-68, wherein the targeting moiety is an antibody or antigen-specific binding fragment.

Item 70. The agent of item 69, wherein the antibody or antigen-specific binding fragment thereof specifically binds a cancer antigen.

Item 71. The agent of any one of items 1-68, wherein the targeting moiety is an aptamer.

Item 72. The agent of item 71, wherein the aptamer specifically binds a cancer antigen.

Item 73. The agent of any one of items 71-72, wherein the aptamer comprises DNA.

Item 74. The agent of any one of items 71-72, wherein the aptamer comprises RNA.

Item 75. The agent of any one of items 73-74, wherein the aptamer is single-stranded.

Item 76. The agent of any one of items 71-75, wherein the aptamer is a target cell-specific aptamer chosen from a random candidate library.

Item 77. The agent of any one of items 71-76, wherein the aptamer is an anti-EGFR aptamer.

Item 78. The agent of any one of items 77, wherein the anti-EGFR aptamer comprises any one of SEQ ID NOs: 95-164.

Item 79. The agent of any one of items 71-78, wherein the aptamer binds to the cancer on the cancer cell with a K_{d} from 1 picomolar to 500 nanomolar.

Item 80. The agent of any one of items 71-79, wherein the aptamer binds to the cancer with a K_{d} from 1 picomolar to 100 nanomolar.

Item 81. The agent of any one of items 1-68, wherein the targeting moiety comprises IL-2, IL-4, IL-6, α-MSH, transferrin, folic acid, EGF, TGF, PD1, IL-13, stem cell factor, insulin-like growth factor (IGF), or CD40.

Item 82. The agent of any one of items 1-68, wherein the targeting moiety comprises a full-length sequence of IL-2, IL-4, IL-6, α-MSH, transferrin, folic acid, EGF, TGF, PD1, IL-13, stem cell factor, insulin-like growth factor (IGF), or CD40.

Item 83. The agent of any one of items 1-68, wherein the targeting moiety comprises a truncated form, analog, variant, or derivative of IL-2, IL-4, IL-6, α-MSH, transferrin, folic acid, EGF, TGF, PD1, IL-13, stem cell factor, insulin-like growth factor (IGF), or CD40.

Item 84. The agent of any one of items 1-68, wherein the targeting moiety binds a target on the cancer comprising IL-2 receptor, IL-4, IL-6, melanocyte stimulating hormone receptor (MSH receptor), transferrin receptor (TR), folate receptor 1 (FOLR), folate hydroxylase (FOLH1), EGF receptor, PD-L1, PD-L2, IL-13R, CXCR4, IGFR, or CD40L.

Item 85. The agent of any one of items 1-84, wherein one immune cell engaging domain comprises a VH domain and the other immune cell engaging domain comprises a VL domain.

Item 86. The agent of any one of items 1-85, wherein the first immune cell binding partner is bound to an inert binding partner and separated from it by a cleavage site.

Item 87. The agent of any one of items 1-86, wherein the second immune cell binding partner is bound to an inert binding partner and separated from it by a cleavage site.

Item 88. The agent of any one of items 1-87, wherein
a. the first immune cell binding partner is bound to an inert binding partner and separated from it by a first cleavage site and
b. the second immune cell binding partner is bound to the inert binding partner and separated from it by a second cleavage site.

Item 89. The agent of item 88, wherein the first cleavage site and the second cleavage site are the same cleavage site.

Item 90. The agent of item 88, wherein the first cleavage site and the second cleavage site are different cleavage sites.

Item 91. The agent of any one of items 1-90, wherein at least one cleavage site is a protease cleavage site.

Item 92. The agent of any one of items 1-91, wherein at least one enzyme expressed by the cancer cells is a protease.

Item 93. The agent of any one of items 1-92, wherein at least one inert binding partner specifically binds the immune cell engaging domain.

Item 94. The agent of item 93, wherein at least one inert binding partner is a VH or VL domain.

Item 95. The agent of item 94, wherein
a. when the immune cell engaging domain is a VH domain, the inert binding partner is a VL domain and
b. when the immune cell engaging domain is VL domain, the inert binding partner is a VH domain.

Item 96. The agent of item 3, wherein the first component is covalently bound to the second component by a linker comprising a cleavage site.

Item 97. The agent of item 96, wherein the cleavage site is a protease cleavage site.

Item 98. The agent of items 97, wherein the protease cleavage site is cleavable in blood.

Item 99. The agent of item 98, wherein the protease cleavage site is a cleavage site for thrombin, neutrophil elastase, or furin.

Item 100. The agent of item 97, wherein the protease cleavage site is cleavable by a tumor-associated protease.

Item 101. The agent of item 100, wherein the tumor-associated protease cleavage site comprises any one of SEQ ID NOs: 1-84.

Item 102. An agent for treating cancer in a patient comprising a selective immune cell binding agent comprising:
a. a first component comprising a targeted immune cell binding agent comprising:
   i. a targeting moiety capable of targeting the cancer;
   ii. a first immune cell engaging domain capable of immune engaging activity when binding a second immune cell engaging domain, wherein the second immune cell engaging domain is not part of the first component;
b. a cleavage site separating the first immune cell engaging domain and an inert binding partner, wherein the cleavage site is:
   i. cleaved by an enzyme expressed by the cancer cells;
   ii. cleaved through a pH-sensitive cleavage reaction inside the cancer cell;
   iii. cleaved by a complement-dependent cleavage reaction; or
   iv. cleaved by a protease that is colocalized to the cancer cell by a targeting moiety that is the same or different from the targeting moiety in the agent,
wherein cleavage of the cleavage site causes loss of the inert binding partner and allows for binding to the second immune cell engaging domain that is not part of the agent.

Item 103. A set of nucleic acid molecules encoding the first and second component of the agent of any one of items 1-101.

Item 104. A nucleic acid molecule encoding the selective immune cell binding agent of item 102.

Item 105. A method of treating cancer in a patient comprising administering the agent of any one of items 1-101.

Item 106. The method of item 105, wherein if the patient has regulatory T cells in the tumor, the selective immune cell binding agent does not target markers present on regulatory immune cells (including, but not limited to CD4 and CD25).

Item 107. The method of any one of items 105-106, wherein the selective immune cell binding agent does not target markers present on TH17 cells.

Item 108. The method of any one of items 105-107, wherein the selective immune cell binding agent activates T cells that will target the tumor cells for lysis.

Item 109. The method of any one of items 105-108, wherein if the patient has regulatory T cells in the tumor, the immune cell selection moiety targets CD8+ T cells by specifically binding CD8.

Item 110. The method of any one of items 105-108, wherein if the patient has regulatory T cells in the tumor, the immune cell selection moiety targets CD8+ T cells and CD4+ T cells by specifically binding CXCR3.

Item 111. The method of any one of items 105-110, wherein the cancer is any one of breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, liver cancer, leukemia, myeloma, nonHodgkin lymphoma, Hodgkin lymphoma, acute myeloid leukemia, acute lymphoblastic leukemia, chronic lymphoblastic leukemia, lymphoproliferative disorder, myelodysplastic disorder, myeloproliferative disease or premalignant disease.

Item 112. A method of targeting an immune response of a patient to cancer comprising administering the agent of any one of items 1-101 to the patient.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. An agent for treating cancer in a patient comprising:
a. a first component comprising a targeted immune cell binding agent comprising:
i. a targeting moiety capable of targeting the cancer;
ii. a first immune cell engaging domain capable of immune engaging activity when binding a second immune cell engaging domain, wherein the second immune cell engaging domain is not part of the first component;
b. a second component comprising a selective immune cell binding agent comprising:
i. an immune cell selection moiety capable of selectively targeting an immune cell;
ii. a second immune cell engaging domain capable of immune cell engaging activity when binding the first immune cell engaging domain, wherein the first and second immune cell engaging domains are capable of binding when neither is bound to an inert binding partner,
wherein at least one of the first immune cell engaging domain or the second immune cell engaging domain is bound to an inert binding partner such that the first and second immune cell engaging domains are not bound to each other unless the inert binding partner is removed; and
further comprising a cleavage site separating an inert binding partner and the immune cell engaging domain to which it binds, wherein the cleavage site is:
i. cleaved by an enzyme expressed by the cancer cells;
iii. cleaved through a pH-sensitive cleavage reaction inside the cancer cell;
iv. cleaved by a complement-dependent cleavage reaction; or
v. cleaved by a protease that is colocalized to the cancer cell by a targeting moiety that is the same or different from the targeting moiety in the agent.

2. The agent of claim 1, wherein the first component is not covalently bound to the second component.

3. The agent of claim 1, wherein the first component is covalently bound to the second component.

4. The agent of claim 1, wherein the immune cell engaging domains, when bound to each other, are capable of binding an antigen expressed on the surface of the immune cell.

5. The agent of claim 1, wherein the immune cell selection moiety capable of selectively targeting an immune cell selectively targets a T cell, a macrophage, a natural killer cell, a neutrophil, an eosinophil, a basophil, a γδ T cell, a natural killer T cell (NKT cells), or an engineered immune cell.

6. The agent of claim 5, wherein the immune cell selection moiety capable of selectively targeting an immune cell selectively targets a T cell, optionally where the T cell is a CD8+ or CD4+ T cell.

7. The agent of claim 1, wherein the immune cell selection moiety targets CD8, CD4, or CXCR3, or does not specifically bind regulatory T cells.

8. The agent of claim 1, wherein the immune cell engaging domains, when bound to each other, are capable of binding CD3 or TCR.

9. The agent of claim 1, wherein the immune cell selection moiety comprises an aptamer or an antibody or antigen-specific binding fragment thereof, optionally wherein the aptamer or antibody or antigen-specific binding fragment thereof specifically binds an antigen on a T cell.

10. The agent of claim 1, wherein the targeting moiety is an aptamer or antibody or antigen-specific binding fragment, optionally wherein the aptamer or antibody or antigen-specific binding fragment thereof specifically binds a cancer antigen.

11. The agent of claim 1, wherein the targeting moiety binds a target on the cancer comprising IL-2 receptor, IL-4, IL-6, melanocyte stimulating hormone receptor (MSH receptor), transferrin receptor (TR), folate receptor 1 (FOLR), folate hydroxylase (FOLH1), EGF receptor, PD-L1, PD-L2, IL-13R, CXCR4, IGFR, or CD40L.

12. The agent of claim 1, wherein one immune cell engaging domain comprises a VH domain and the other immune cell engaging domain comprises a VL domain, optionally wherein at least one inert binding partner is a VH or VL domain.

13. The agent of claim 1, wherein the first immune cell engaging domain and/or second immune cell engaging domain is bound to an inert binding partner and separated from it by a cleavage site, optionally wherein at least one cleavage site is a protease cleavage site.

14. The agent of claim 13, wherein
a. when the immune cell engaging domain is a VH domain, the inert binding partner is a VL domain and
b. when the immune cell engaging domain is VL domain, the inert binding partner is a VH domain.

15. The agent of claim 3, wherein the first component is covalently bound to the second component by a linker comprising a cleavage site, optionally wherein the cleavage site is a protease cleavage site.

16. An agent for use in a kit or composition for treating cancer comprising a selective immune cell binding agent comprising:
a. a first component comprising a targeted immune cell binding agent comprising:
i. a targeting moiety capable of targeting the cancer;
ii. a first immune cell engaging domain capable of immune engaging activity when binding a second immune cell engaging domain, wherein the second immune cell engaging domain is not part of the first component;
iii. a cleavage site separating the first immune cell engaging domain and an inert binding partner, wherein the cleavage site is:
1. cleaved by an enzyme expressed by the cancer cells;
2. cleaved through a pH-sensitive cleavage reaction inside the cancer cell;
3. cleaved by a complement-dependent cleavage reaction; or
4. cleaved by a protease that is colocalized to the cancer cell by a targeting moiety that is the same or different from the targeting moiety in the agent,
wherein cleavage of the cleavage site causes loss of the inert binding partner and allows for binding to the second immune cell engaging domain that is not part of the agent.

17. A set of nucleic acid molecules encoding the first and second component of the agent of claim 1.

18. A method of treating cancer in a patient comprising administering the agent of claim 1, optionally wherein the cancer is any one of breast cancer, ovarian cancer, endometrial cancer, cervical cancer, bladder cancer, renal cancer, melanoma, lung cancer, prostate cancer, testicular cancer, thyroid cancer, brain cancer, esophageal cancer, gastric cancer, pancreatic cancer, colorectal cancer, liver cancer, leukemia, myeloma, nonHodgkin lymphoma, Hodgkin lymphoma, acute myeloid leukemia, acute lymphoblastic leukemia, chronic lymphoblastic leukemia, lymphoproliferative disorder, myelodysplastic disorder, myeloproliferative disease or premalignant disease.

19. The method of claim 18, wherein if the patient has regulatory T cells in the tumor, the selective immune cell binding agent does not target markers present on regulatory immune cells (including, but not limited to CD4 and CD25).

20. A method of targeting an immune response of a patient to cancer comprising administering the agent of claim 1 to the patient.


SEQUENCE LISTING (172 sequences)

SEQ ID NO: 1 - protein, 8 aa, Artificial Sequence
  Synthetic ADAM28 cleavage site
KPAKFFRL

SEQ ID NO: 2 - protein, 8 aa, Artificial Sequence
  Synthetic ADAM28 cleavage site
DPAKFFRL

SEQ ID NO: 3 - protein, 8 aa, Artificial Sequence
  Synthetic ADAM28 cleavage site
KPMKFFRL

SEQ ID NO: 4 - protein, 8 aa, Artificial Sequence
  Synthetic ADAM28 cleavage site
LPAKFFRL

SEQ ID NO: 5 - protein, 8 aa, Artificial Sequence
  Synthetic ADAM28 cleavage site
LPMKFFRL

SEQ ID NO: 6 - protein, 8 aa, Artificial Sequence
  Synthetic ADAM28 cleavage site
KPAMFFRL

SEQ ID NO: 7 - protein, 8 aa, Artificial Sequence
  Synthetic ADAM28 cleavage site
YPAKFFRL

SEQ ID NO: 8 - protein, 8 aa, Artificial Sequence
  Synthetic ADAM28 cleavage site
KWAKFFRL

SEQ ID NO: 9 - protein, 8 aa, Artificial Sequence
  Synthetic ADAM28 cleavage site
DPMKFFRL

SEQ ID NO: 10 - protein, 8 aa, Artificial Sequence
  Synthetic ADAM28 cleavage site
DPAMFFRL

SEQ ID NO: 11 - protein, 8 aa, Artificial Sequence
  Synthetic ADAM28 cleavage site
DPMMFFRL

SEQ ID NO: 12 - protein, 8 aa, Artificial Sequence
  Synthetic ADAM28 cleavage site
KMAMFFRL

SEQ ID NO: 13 - protein, 8 aa, Artificial Sequence
  Synthetic ADAM28 cleavage site
KMAMFFIM

SEQ ID NO: 14 - protein, 8 aa, Artificial Sequence
  Synthetic ADAM28 cleavage site
KPAMFFIM

SEQ ID NO: 15 - protein, 8 aa, Artificial Sequence
  Synthetic ADAM28 cleavage site
LPAMFFRL

SEQ ID NO: 16 - protein, 8 aa, Artificial Sequence
  Synthetic ADAM28 cleavage site
LPMMFFRL

SEQ ID NO: 17 - protein, 8 aa, Artificial Sequence
  Synthetic ADAM28 cleavage site
LMAMFFRL

SEQ ID NO: 18 - protein, 8 aa, Artificial Sequence
  Synthetic ADAM28 cleavage site
LMAMFFIM

SEQ ID NO: 19 - protein, 8 aa, Artificial Sequence
  Synthetic ADAM28 cleavage site
LPAMFFIM

SEQ ID NO: 20 - protein, 8 aa, Artificial Sequence
  Synthetic ADAM28 cleavage site
LPAMFFYM

SEQ ID NO: 21 - protein, 8 aa, Artificial Sequence
  Synthetic ADAM28 cleavage site
KPMMFFRL

SEQ ID NO: 22 - protein, 8 aa, Artificial Sequence
  Synthetic ADAM28 cleavage site
KPAKFFYM

SEQ ID NO: 23 - protein, 8 aa, Artificial Sequence
  Synthetic ADAM28 cleavage site
KPAKFFIM

SEQ ID NO: 24 - protein, 8 aa, Artificial Sequence
  Synthetic ADAM28 cleavage site
IPMKFFRL

SEQ ID NO: 25 - protein, 8 aa, Artificial Sequence
  Synthetic ADAM28 cleavage site
IPAMFFRL

SEQ ID NO: 26 - protein, 8 aa, Artificial Sequence
  Synthetic ADAM28 cleavage site
IPMMFFRL

SEQ ID NO: 27 - protein, 8 aa, Artificial Sequence
  Synthetic ADAM28 cleavage site
IMAMFFRL

SEQ ID NO: 28 - protein, 8 aa, Artificial Sequence
  Synthetic ADAM28 cleavage site
IMAMFFIM

SEQ ID NO: 29 - protein, 8 aa, Artificial Sequence
  Synthetic ADAM28 cleavage site
IPAMFFIM

SEQ ID NO: 30 - protein, 8 aa, Artificial Sequence
  Synthetic ADAM28 cleavage site
IPAMFFYM

SEQ ID NO: 31 - protein, 2 aa, Artificial Sequence
  Synthetic cathepsin B cleavage site
FR

SEQ ID NO: 32 - protein, 2 aa, Artificial Sequence
  Synthetic cathepsin B cleavage site
FK

SEQ ID NO: 33 - protein, 2 aa, Artificial Sequence
  Synthetic cathepsin B cleavage site
VA

SEQ ID NO: 34 - protein, 2 aa, Artificial Sequence
  Synthetic cathepsin B cleavage site
VR

SEQ ID NO: 35 - protein, 1 aa, Artificial Sequence
  Synthetic cathepsin B cleavage site
  MOD_RES (1)..(1): citrulline
V

SEQ ID NO: 36 - protein, 8 aa, Artificial Sequence
  Synthetic cathepsin B cleavage site
HLVEALYL

SEQ ID NO: 37 - protein, 12 aa, Artificial Sequence
  Synthetic cathepsin B cleavage site
SLLKSRMVPNFN

SEQ ID NO: 38 - protein, 12 aa, Artificial Sequence
  Synthetic cathepsin B cleavage site
SLLIARRMPNFN

SEQ ID NO: 39 - protein, 4 aa, Artificial Sequence
  Synthetic cathepsin B cleavage site
KKFA

SEQ ID NO: 40 - protein, 4 aa, Artificial Sequence
  Synthetic cathepsin B cleavage site
AFKK

SEQ ID NO: 41 - protein, 3 aa, Artificial Sequence
  Synthetic cathepsin B cleavage site
QQQ

SEQ ID NO: 42 - protein, 9 aa, Artificial Sequence
  Synthetic cathepsin D cleavage site
PRSFFRLGK

SEQ ID NO: 43 - protein, 12 aa, Artificial Sequence
  Synthetic cathepsin D cleavage site
SGVVIATVIVIT

SEQ ID NO: 44 - protein, 3 aa, Artificial Sequence
  Synthetic cathepsin K cleavage site
GGP

SEQ ID NO: 45 - protein, 12 aa, Artificial Sequence
  Synthetic MMP1 cleavage site
SLGPQGIWGQFN

SEQ ID NO: 46 - protein, 7 aa, Artificial Sequence
  Synthetic MMP2 cleavage site
AIPVSLR

SEQ ID NO: 47 - protein, 12 aa, Artificial Sequence
  Synthetic MMP2 cleavage site
SLPLGLWAPNFN

SEQ ID NO: 48 - protein, 8 aa, Artificial Sequence
  Synthetic MMP2 cleavage site
HPVGLLAR

SEQ ID NO: 49 - protein, 8 aa, Artificial Sequence
  Synthetic MMP2 cleavage site
GPLGVRGK

SEQ ID NO: 50 - protein, 8 aa, Artificial Sequence
  Synthetic MMP2 cleavage site
GPLGLWAQ

SEQ ID NO: 51 - protein, 8 aa, Artificial Sequence
  Synthetic MMP3 cleavage site
STAVIVSA

SEQ ID NO: 52 - protein, 8 aa, Artificial Sequence
  Synthetic MMP7 cleavage site
GPLGLARK

SEQ ID NO: 53 - protein, 8 aa, Artificial Sequence
  Synthetic MMP7 cleavage site
RPLALWRS

SEQ ID NO: 54 - protein, 12 aa, Artificial Sequence
  Synthetic MMP7 cleavage site
SLRPLALWRSFN

SEQ ID NO: 55 - protein, 6 aa, Artificial Sequence
  Synthetic MMP2/9 cleavage site
GILGVP

SEQ ID NO: 56 - protein, 8 aa, Artificial Sequence
  Synthetic MMP2/9 cleavage site
GPLGIAGQ

SEQ ID NO: 57 - protein, 8 aa, Artificial Sequence
  Synthetic MMP9 cleavage site
AVRWLLTA

SEQ ID NO: 58 - protein, 7 aa, Artificial Sequence
  Synthetic MMP9 cleavage site
PLGLYAL

SEQ ID NO: 59 - protein, 9 aa, Artificial Sequence
  Synthetic MMP9 cleavage site
GPQGIAGQR

SEQ ID NO: 60 - protein, 8 aa, Artificial Sequence
  Synthetic MMP9 cleavage site
KPVSLSYR

SEQ ID NO: 61 - protein, 8 aa, Artificial Sequence
  Synthetic MMP11 cleavage site
AAATSIAM

SEQ ID NO: 62 - protein, 8 aa, Artificial Sequence
  Synthetic MMP11 cleavage site
AAGAMFLE

SEQ ID NO: 63 - protein, 12 aa, Artificial Sequence
  Synthetic MMP13 cleavage site
GPQGLAGQRGIV

SEQ ID NO: 64 - protein, 5 aa, Artificial Sequence
  Synthetic MMP14 cleavage site
PRHLR

SEQ ID NO: 65 - protein, 12 aa, Artificial Sequence
  Synthetic MMP14 cleavage site
PQGLLGAPGILG

SEQ ID NO: 66 - protein, 8 aa, Artificial Sequence
  Synthetic MMP14 cleavage site
PRSAKELR

SEQ ID NO: 67 - protein, 6 aa, Artificial Sequence
  Synthetic PSA / KLK3
HSSKLQ

SEQ ID NO: 68 - protein, 5 aa, Artificial Sequence
  Synthetic PSA / KLK3
SSKLQ

SEQ ID NO: 69 - protein, 4 aa, Artificial Sequence
  Synthetic KLK4
RQQR

SEQ ID NO: 70 - protein, 3 aa, Artificial Sequence
  Synthetic TMPRSS2
GGR

SEQ ID NO: 71 - protein, 3 aa, Artificial Sequence
  Synthetic Legumain
AAN

SEQ ID NO: 72 - protein, 3 aa, Artificial Sequence
  Synthetic ST14 (Matriptase)
QAR

SEQ ID NO: 73 - protein, 8 aa, Artificial Sequence
  Synthetic C1s cleavage site
YLGRSYKV

SEQ ID NO: 74 - protein, 6 aa, Artificial Sequence
  Synthetic C1s cleavage site
  misc_feature (6)..(6): Xaa can be any naturally occurring amino acid
MQLGRX

SEQ ID NO: 75 - protein, 8 aa, Artificial Sequence
  Synthetic MASP2 cleavage site
SLGRKIQI

SEQ ID NO: 76 - protein, 9 aa, Artificial Sequence
  Synthetic C2a and Bb cleavage site
GLARSNLDE

SEQ ID NO: 77 - protein, 8 aa, Artificial Sequence
  Synthetic uPa cleavage site
TYSRSRYL

SEQ ID NO: 78 - protein, 12 aa, Artificial Sequence
  Synthetic uPa cleavage site
KKSPGRVVGGSV

SEQ ID NO: 79 - protein, 8 aa, Artificial Sequence
  Synthetic uPa cleavage site
NSGRAVTY

SEQ ID NO: 80 - protein, 3 aa, Artificial Sequence
  Synthetic uPa cleavage site
AFK

SEQ ID NO: 81 - protein, 12 aa, Artificial Sequence
  Synthetic tissue-type plasminogen activator (tPA)
GGSGQRGRKALE

SEQ ID NO: 82 - protein, 10 aa, Artificial Sequence
  Synthetic ADAM10
PRYEAYKMGK

SEQ ID NO: 83 - protein, 5 aa, Artificial Sequence
  Synthetic ADAM12
LAQAF

SEQ ID NO: 84 - protein, 11 aa, Artificial Sequence
  Synthetic ADAM17
EHADLLAVVAK

SEQ ID NO: 85 - protein, 5 aa, Artificial Sequence
  Synthetic flexible amino acid linker (may be presented in repeating fashion)
GGGGS

SEQ ID NO: 86 - protein, 4 aa, Artificial Sequence
  Synthetic flexible amino acid linker (may be presented in repeating fashion)
GGGS

SEQ ID NO: 87 - protein, 2 aa, Artificial Sequence
  Synthetic flexible amino acid linker (may be presented in repeating fashion)
GS

SEQ ID NO: 88 - protein, 5 aa, Artificial Sequence
  Synthetic flexible amino acid linker (may be presented in repeating fashion)
GSGGS

SEQ ID NO: 89 - protein, 4 aa, Artificial Sequence
  Synthetic flexible amino acid linker (may be presented in repeating fashion)
GGSG

SEQ ID NO: 90 - protein, 5 aa, Artificial Sequence
  Synthetic flexible amino acid linker (may be presented in repeating fashion)
GGSGG

SEQ ID NO: 91 - protein, 5 aa, Artificial Sequence
  Synthetic flexible amino acid linker (may be presented in repeating fashion)
GSGSG

SEQ ID NO: 92 - protein, 5 aa, Artificial Sequence
  Synthetic flexible amino acid linker (may be presented in repeating fashion)
GSGGG

SEQ ID NO: 93 - protein, 5 aa, Artificial Sequence
  Synthetic flexible amino acid linker (may be presented in repeating fashion)
GGGSG

SEQ ID NO: 94 - protein, 5 aa, Artificial Sequence
  Synthetic flexible amino acid linker (may be presented in repeating fashion)
GSSSG

SEQ ID NO: 95 - RNA, 51 nt, Artificial Sequence
  Synthetic Anti-EGFR aptamer (tight binder with Kd=2.4 nM)
ugccgcuauaaugcacggauuuaaucgccguagaaaagcaugucaaagccg

SEQ ID NO: 96 - RNA, 51 nt, Artificial Sequence
  Synthetic Anti-EGFR aptamer
uggcgcuaaauagcacggaaauaaucgccguagaaaagcaugucaaagccg

SEQ ID NO: 97 - RNA, 51 nt, Artificial Sequence
  Synthetic Anti-EGFR aptamer
ugcuaguauaucgcacggauuuaaucgccguagaaaagcaugucaaagccg

SEQ ID NO: 98 - RNA, 51 nt, Artificial Sequence
  Synthetic Anti-EGFR aptamer
ugccgccauaucacacggauuuaaucgccguagaaaagcaugucaaagccg

SEQ ID NO: 99 - RNA, 51 nt, Artificial Sequence
  Synthetic Anti-EGFR aptamer
uuccgcuguauaacacggacuuaaucgccguaguaaagcaugucaaagccg

SEQ ID NO: 100 - RNA, 51 nt, Artificial Sequence
  Synthetic Anti-EGFR aptamer
ugucgcucuauugcacggauuuaaucgccguagaaaagcaugucaaagccg

SEQ ID NO: 101 - RNA, 51 nt, Artificial Sequence
  Synthetic Anti-EGFR aptamer
ugcugcuuuaucccacauauuuuuuccccucauaacaauauuucucccccc

SEQ ID NO: 102 - RNA, 51 nt, Artificial Sequence
  Synthetic Anti-EGFR aptamer
  misc_feature (4)..(4): n is a, c, g, or u
  misc_feature (15)..(15): n is a, c, g, or u
  misc_feature (45)..(45): n is a, c, g, or u
  misc_feature (47)..(47): n is a, c, g, or u
ugcngcuauaucgcncguauuuaaucgccguagaaaagcaugucnangccg

SEQ ID NO: 103 - RNA, 51 nt, Artificial Sequence
  Synthetic Anti-EGFR aptamer
ugcaaagaaaacgcacguauuuaaucgccguaguaaagcaugucaaagccg

SEQ ID NO: 104 - RNA, 53 nt, Artificial Sequence
  Synthetic Anti-EGFR aptamer
ugcaucacuaucgaaccuauuuaauccaccaaaauaauugcaaguccauacuc

SEQ ID NO: 105 - RNA, 51 nt, Artificial Sequence
  Synthetic Anti-EGFR aptamer
  misc_feature (5)..(6): n is a, c, g, or u
  misc_feature (17)..(17): n is a, c, g, or u
  misc_feature (48)..(48): n is a, c, g, or u
ugccnnaauaacacacnuauauaaucgccguacaaaaucaugucaaanccg

SEQ ID NO: 106 - RNA, 51 nt, Artificial Sequence
  Synthetic Anti-EGFR aptamer
ugcagcuguauugcacguauuuaaucgccguagaaaagcaugucaaagccg

SEQ ID NO: 107 - RNA, 50 nt, Artificial Sequence
  Synthetic Anti-EGFR aptamer
uuccgauaaucccgcguacuaaaucaccauagucaacaauuuccaaccuc

SEQ ID NO: 108 - RNA, 50 nt, Artificial Sequence
  Synthetic Anti-EGFR aptamer
uccacuauaucacacguauuuaaucgccguagaaaagcaugucaaagccg

SEQ ID NO: 109 - RNA, 51 nt, Artificial Sequence
  Synthetic Anti-EGFR aptamer
ucccucaaccucgcuacuauuuaaucgccguagaaaagcaugucaaagccu

SEQ ID NO: 110 - RNA, 51 nt, Artificial Sequence
  Synthetic Anti-EGFR aptamer
ugccgcuauaucacacgaauuuaaucgccguagaaaagcaugucaaagccg

SEQ ID NO: 111 - RNA, 51 nt, Artificial Sequence
  Synthetic Anti-EGFR aptamer
agccccuagaacacacggauuuaaucgccguagaaaagcaugucaaagccg

SEQ ID NO: 112 - RNA, 51 nt, Artificial Sequence
  Synthetic Anti-EGFR aptamer
ugccaauauauaacacggaauuaaucgccguagaaaagcaugucaaagccg

SEQ ID NO: 113 - RNA, 51 nt, Artificial Sequence
  Synthetic Anti-EGFR aptamer
ugccgcuauagcgcacggauuuaaucgccguagaaaagcaugucaaagccg

SEQ ID NO: 114 - RNA, 50 nt, Artificial Sequence
  Synthetic Anti-EGFR aptamer
ugcagauauaugucacucauuaauccccguauaaaaacauaacuaagcuc

SEQ ID NO: 115 - RNA, 51 nt, Artificial Sequence
  Synthetic Anti-EGFR aptamer
uguagcuguauugcacacauuaaaucgccguaguaaagcaugucaaagccg

SEQ ID NO: 116 - RNA, 50 nt, Artificial Sequence
  Synthetic Anti-EGFR aptamer
uaccaauauaucgccacacauaaucgccguagaaaagcaugucaaagccg

SEQ ID NO: 117 - RNA, 51 nt, Artificial Sequence
  Synthetic Anti-EGFR aptamer
ugccgcuaugcccacggaauuuaaucgccguagaaaaacaugucaaagucg

SEQ ID NO: 118 - RNA, 51 nt, Artificial Sequence
  Synthetic Anti-EGFR aptamer
ugccgcuauuuagcacggauuaaaucgccguagaaaagcaugucaaagccg

SEQ ID NO: 119 - RNA, 51 nt, Artificial Sequence
  Synthetic Anti-EGFR aptamer
  misc_feature (45)..(45): n is a, c, g, or u
ugccgcuauuuagcacggauuaaaucgccguagaaaagcaugucnaagccg

SEQ ID NO: 120 - RNA, 51 nt, Artificial Sequence
  Synthetic Anti-EGFR aptamer
  misc_feature (41)..(41): n is a, c, g, or u
uguaguaauaugacacggauuuaaucgccguagaaaagcangucaaagccu

SEQ ID NO: 121 - RNA, 51 nt, Artificial Sequence
  Synthetic Anti-EGFR aptamer
ugucgccauuacgcacggauuuaaucgccguagaaaagcaugucaaagccg

SEQ ID NO: 122 - RNA, 51 nt, Artificial Sequence
  Synthetic Anti-EGFR aptamer
ugcccccaaacuacacaaauuuaaucgccguauaaaagcaugucaaagccg

SEQ ID NO: 123 - RNA, 49 nt, Artificial Sequence
  Synthetic Anti-EGFR aptamer
ugcacuaucucacacguacuaaucgccguauaaaagcaugucaaagccg

SEQ ID NO: 124 - RNA, 51 nt, Artificial Sequence
  Synthetic Anti-EGFR aptamer
ugucgcaauaauacacuaauuuaaucgccguagaaaagcaugucaaagccg

SEQ ID NO: 125 - RNA, 49 nt, Artificial Sequence
  Synthetic Anti-EGFR aptamer
ugcaacaauauagcacguauuuaaucgccguaguaaagcaugucaaagg

SEQ ID NO: 126 - RNA, 51 nt, Artificial Sequence
  Synthetic Anti-EGFR aptamer
cuaccacaaaucccacauauuuaaucucccaaucaaaucuuguccauuccc

SEQ ID NO: 127 - RNA, 51 nt, Artificial Sequence
  Synthetic Anti-EGFR aptamer
ugcccuaaacucacacggauauaaucgccguagaaaagcaugucaaagccg

SEQ ID NO: 128 - RNA, 51 nt, Artificial Sequence
  Synthetic Anti-EGFR aptamer
uugucguaugucacacguauuaaaucgccguauaaaagcaugucaaagccg

SEQ ID NO: 129 - RNA, 51 nt, Artificial Sequence
  Synthetic Anti-EGFR aptamer
uuccgcuauaacacacggagaaaaucgccguaguaaagcaugucaaagccg

SEQ ID NO: 130 - RNA, 51 nt, Artificial Sequence
  Synthetic Anti-EGFR aptamer
ugccgauauaacgcacggauauaaucgccguagaaaagcaugucaaagccg

SEQ ID NO: 131 - RNA, 51 nt, Artificial Sequence
  Synthetic Anti-EGFR aptamer
ugccauuauacagcacggauuuaaucgccguagaaaagcaugucaaagccg

SEQ ID NO: 132 - RNA, 50 nt, Artificial Sequence
  Synthetic Anti-EGFR aptamer
uccagaaauaugcacacauuuaaucgccguagaaaagcaugucaaagccg

SEQ ID NO: 133 - RNA, 50 nt, Artificial Sequence
  Synthetic Anti-EGFR aptamer
uccgcuaaacaacacggauacaaucgccguagaaaagcauguccaagccg

SEQ ID NO: 134 - RNA, 49 nt, Artificial Sequence
  Synthetic Anti-EGFR aptamer
  misc_feature (46)..(48): n is a, c, g, or u
ugcacuaucucacacguacuaaucgccguauaaaagcaugucaaannng

SEQ ID NO: 135 - RNA, 50 nt, Artificial Sequence
  Synthetic Anti-EGFR aptamer
  misc_feature (3)..(3): n is a, c, g, or u
  misc_feature (6)..(6): n is a, c, g, or u
  misc_feature (8)..(10): n is a, c, g, or u
  misc_feature (21)..(21): n is a, c, g, or u
  misc_feature (45)..(45): n is a, c, g, or u
aungcnannnuacacguauunaaucgccguagaaaagcaugucanagccg

SEQ ID NO: 136 - RNA, 51 nt, Artificial Sequence
  Synthetic Anti-EGFR aptamer
ugcugcuauauugcaauuuuuuaaacuaaguagaaaaccauguacaagucg

SEQ ID NO: 137 - RNA, 51 nt, Artificial Sequence
  Synthetic Anti-EGFR aptamer
ugucgccauauugcacggauuuaaucgccguagaaaagcauguccaagccg

SEQ ID NO: 138 - RNA, 52 nt, Artificial Sequence
  Synthetic Anti-EGFR aptamer
ugccguuauaacccacggaauuuaaccuccguagaaaagcaugucaaagccg

SEQ ID NO: 139 - RNA, 52 nt, Artificial Sequence
  Synthetic Anti-EGFR aptamer
  misc_feature (40)..(40): n is a, c, g, or u
ugugaauauauaucacggauuuaaucgccguauaaaagcnaugucaaagccg

SEQ ID NO: 140 - RNA, 51 nt, Artificial Sequence
  Synthetic Anti-EGFR aptamer
  misc_feature (10)..(11): n is a, c, g, or u
  misc_feature (13)..(13): n is a, c, g, or u
ugccgauaunnancacggauuuaaucgccguagaaaagcauguccaagccg

SEQ ID NO: 141 - RNA, 50 nt, Artificial Sequence
  Synthetic Anti-EGFR aptamer
ugucacuaaauugcacguauauaaucgccguaguaagcaugucaaagccg

SEQ ID NO: 142 - RNA, 51 nt, Artificial Sequence
  Synthetic Anti-EGFR aptamer
ugcaaccauaaagcacguaauaaaucgccguauauaagcaugucaaagccg

SEQ ID NO: 143 - RNA, 50 nt, Artificial Sequence
  Synthetic Anti-EGFR aptamer
ugccgcuauauagcacguauuaaucgccguaguaaagcaugucaaagccg

SEQ ID NO: 144 - RNA, 52 nt, Artificial Sequence
  Synthetic Anti-EGFR aptamer
ugccgcuauagcacacggaauuuaaucgccguaguaaagcaugucaaagccg

SEQ ID NO: 145 - RNA, 51 nt, Artificial Sequence
  Synthetic Anti-EGFR aptamer
  misc_feature (15)..(15): n is a, c, g, or u
  misc_feature (45)..(45): n is a, c, g, or u
ugcagguauauaacncggauuuaaucgccguagaaaagcaugucnaagccg

SEQ ID NO: 146 - RNA, 51 nt, Artificial Sequence
  Synthetic Anti-EGFR aptamer
ugcuccuauaacacacggauuuaaucgccguagaaaagcauguccaagccg

SEQ ID NO: 147 - RNA, 51 nt, Artificial Sequence
  Synthetic Anti-EGFR aptamer
ugcccguaauugcacggauuuaaucgccguagaaaagcauguccaagccgg

SEQ ID NO: 148 - RNA, 51 nt, Artificial Sequence
  Synthetic Anti-EGFR aptamer
  misc_feature (12)..(12): n is a, c, g, or u
  misc_feature (44)..(44): n is a, c, g, or u
acucccuauaungcaacuacauaaucgccguaaauaagcauguncaagccg

SEQ ID NO: 149 - RNA, 54 nt, Artificial Sequence
  Synthetic Anti-EGFR aptamer
ugaagcuagaucacacuaaauuaaucgccguagaaaagcaugucaaaaaagccg

SEQ ID NO: 150 - RNA, 52 nt, Artificial Sequence
  Synthetic Anti-EGFR aptamer
ugacucuuuaucccccguacauuauucaccgaaccaaagcauuaccaucccc

SEQ ID NO: 151 - RNA, 51 nt, Artificial Sequence
  Synthetic Anti-EGFR aptamer
ugacgcccuaacacacguauauaaucgccguagaaaagcaugucaaagccg

SEQ ID NO: 152 - RNA, 51 nt, Artificial Sequence
  Synthetic Anti-EGFR aptamer
ugucgcaaaauagcacguauuuaaucgccguagaaaagcauguccaagccg

SEQ ID NO: 153 - RNA, 51 nt, Artificial Sequence
  Synthetic Anti-EGFR aptamer
ugaguguauaauucacguauuuaaucgccguagaaaagcaugucaaagccg

SEQ ID NO: 154 - RNA, 51 nt, Artificial Sequence
  Synthetic Anti-EGFR aptamer
ugcuacuauaucguagguaacuaaucgcccuacaaacucacucuaaaaccg

SEQ ID NO: 155 - RNA, 53 nt, Artificial Sequence
  Synthetic Anti-EGFR aptamer
uuacgcuauaucacacggaauuuuaaucgccguagaaaagcauguccaagccg

SEQ ID NO: 156 - RNA, 51 nt, Artificial Sequence
  Synthetic Anti-EGFR aptamer
cccaucuguacuacaggaauuuaaucgccguagaaaagcauguccaagccg

SEQ ID NO: 157 - RNA, 51 nt, Artificial Sequence
  Synthetic Anti-EGFR aptamer
ugcccauaaauagcacggauuuaaucgccguagaaaagcauguccaagccg

SEQ ID NO: 158 - RNA, 51 nt, Artificial Sequence
  Synthetic Anti-EGFR aptamer
ugccgcaauaacauacacauauaaucgccguagaaaagcaugucaaagccg

SEQ ID NO: 159 - RNA, 51 nt, Artificial Sequence
  Synthetic Anti-EGFR aptamer
ugcaacuauaucgcacguauguaaucgccguagaaaaagcaugucaaagcc

SEQ ID NO: 160 - RNA, 51 nt, Artificial Sequence
  Synthetic Anti-EGFR aptamer
uuccgcuauauagcacggaauuaaucgccguagaaaagcauguccaagccg

SEQ ID NO: 161 - RNA, 51 nt, Artificial Sequence
  Synthetic Anti-EGFR aptamer
uuccgcuaagucacacgaaauuaaucgccguagaaaagcauguccaagccg

SEQ ID NO: 162 - RNA, 51 nt, Artificial Sequence
  Synthetic Anti-EGFR aptamer
uguagcaauaucacacguaauuaaucgccguauauaagcaugucaaagccg

SEQ ID NO: 163 - RNA, 51 nt, Artificial Sequence
  Synthetic Anti-EGFR aptamer
ugccguuauauaucacggauuuaaucgccguagaaaagcauguccaagccg

SEQ ID NO: 164 - RNA, 49 nt, Artificial Sequence
  Synthetic Anti-EGFR aptamer
uaacacauauaucaaguaacuuaucuccuuaguaaccaucuccaagccg

SEQ ID NO: 165 - protein, 525 aa, Artificial Sequence
  Synthetic Anti-FITC-CD3 VL ATTAC/TEAC component (Anti-Fluorescein scFv with linker between VL-VH 1xG4S connector anti-CD3e VL (20G6) - MMP2 cleavage sequence Ig VH domain  His tag)
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSHHHHHH

SEQ ID NO: 166 - protein, 517 aa, Artificial Sequence
  Synthetic Anti-EpCAM-CD3 VH ATTAC/TEAC component (Anti-EpCAM scFv with 3xG4S linker between VH-VL 1xG4S connector  anti-CD3e VH (20G6) - MMP2 cleavage sequence Ig VL domain  His tag)
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSGEGTSTGSGAIPVSLRGSGGSGGADDIVMTQTPLSLSVTPGQPASISCKSSQSIVHSSGNTYLSWYLQKPGQSPQLLIYKVSNRFSGVPDRFSGSGSGTDFTLKISRVEAEDVGVYYCGQGSHVGPTFGSGTKVEIKHHHHHH

SEQ ID NO: 167 - protein, 518 aa, Artificial Sequence
  Synthetic Anti-EpCAM-CD3 VL ATTAC/TEAC component (Anti-EpCAM scFv with 3xG4S linker between VH-VL 1xG4S connector  anti-CD3e VL (20G6) - MMP2 cleavage sequence Ig VH domain  His tag)
EVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSGGGGSGGGGSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSHHHHHH

SEQ ID NO: 168 - protein, 510 aa, Artificial Sequence
  Synthetic Anti-EpCAM-CD3 scFv (20G6) BiTE construct (anti-EpCAM scFv with 3xG4S linker between VH and VL 1xG4S connector anti-CD3 scFv with linker between VH and VL  His Tag)
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGGSGGSGGADQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSHHHHHH

SEQ ID NO: 169 - protein, 402 aa, Artificial Sequence
  Synthetic Anti-CD8-CD3 VL ATTAC component (Anti-CD8 VHH 1xG4S connector  anti-CD3e VL (20G6) - MMP2 cleavage sequence  Ig VH domain  His tag)  (CD8 targeting VHH domain based upon WO_2017_134306 SEQ ID NO 20)
QVQLQESGGGLVQPGGSLRLSCAASGFTFDDYAMSWVRQVPGKGLEWVSTINWNGGSAEYAEPVKGRFTISRDNAKNTVYLQMNSLKLEDTAVYYCAKDADLVWYNLRTGQGTQVTVSSAAAYPYDVPDYGSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSHHHHHH

SEQ ID NO: 170 - protein, 510 aa, Artificial Sequence
  Synthetic Anti-CD8-CD3 VL ATTAC component (Anti-CD8 scFv with linker between VL-VH  1xG4S connector anti-CD3e VL (20G6) - MMP2 cleavage sequence  Ig VH domain His tag)  (CD8 targeting scFv domain based upon OKT8 antibody)
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSSGGGGSGGGGSGGGGSDVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELKGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSHHHHHH

SEQ ID NO: 171 - protein, 519 aa, Artificial Sequence
  Synthetic Anti-CD4-CD3 VL ATTAC component (Anti-CD4 scFv with linker between VL-VH  1xG4S connector anti-CD3e VL (20G6) - MMP2 cleavage sequence  Ig VH domain His tag)  (CD4 targeting scFv domain based upon Ibalizumab antibody)
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSGGGGSGGGGSGGGGSDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGAIPVSLRGSGGSGGADQVQLVESGGGVVQPGRSLRLSCAASGFTFSSYGMHWVRQAPGKQLEWVAQISFDGSNKYYADSVKGRFTISRDDSKNTLYLQMNSLRAEDTAVYYCASERGHYYDSSAFDYWGQGTLVTVSSHHHHHH

SEQ ID NO: 172 - protein, 457 aa, Artificial Sequence
  Synthetic Anti-CD8-CD3 VL ATTAC component (Anti-CD8 VHH 6xG4S connector  anti-CD3e VL (20G6) - Enterokinase cleavage sequence Ig VH domain  His tag)  (CD8 targeting VHH domain based upon WO_2017_134306 SEQ ID NO 21)
QVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSAAAYPYDVPDYGSGGGGSGGGGSGGGGSGGGGSGGGGSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGGGGGSGGGGSDDDDKGGGGSGGGGSGSGGSGGADQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGSHHHHHH